(19) United States
(12) Patent Application Publication
(10) Pub. No.: US 2026/0107626 A1
(43) Pub. Date: Apr. 16, 2026

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Yeong Ran SONG, Cheonan-si (KR); Jung Geun LEE, Cheonan-si (KR); Nam Heon KIM, Cheonan-si (KR)

(73) Assignee: DUK SAN NEOLUX CO., LTD., Cheonan-si (KR)

(21) Appl. No.: 19/347,913

(22) Filed: Oct. 2, 2025

(30) Foreign Application Priority Data
Oct. 14, 2024 (KR) 10-2024-0139317
Jan. 14, 2025 (KR) 10-2025-0005529

(51) Int. Cl. H10K 50/11; C07D 307/91; H10K 50/19; H10K 85/60

(52) U.S. Cl. CPC H10K 50/11; C07D 307/91; H10K 50/19; H10K 85/622; H10K 85/626; H10K 85/636; H10K 85/6572; H10K 85/6574; H10K 85/6576

(57) ABSTRACT

Provided are a compound of Formula 1 that improves the luminous efficiency, stability, and lifespan of an organic electronic element employing the same, a composition comprising the same, an organic electronic element employing the same, and an electronic device thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer etc.

Materials used as organic material layers in organic electronic elements can be classified according to their function into light-emitting materials and charge transport materials, such as hole injection materials, hole transport materials, electron transport materials, and electron injection materials.

The biggest issues with organic light emitting devices are their lifespan and efficiency, and as displays become larger in size, these efficiency and lifespan issues must be resolved.

Efficiency, lifespan, and driving voltage are interrelated. As efficiency increases, the driving voltage decreases relatively. As the driving voltage decreases, the crystallization of organic materials due to Joule heating generated during driving decreases, which results in a tendency for the lifespan to increase.

However, simply improving the organic material layers does not maximize efficiency. This is because long life and high efficiency can be achieved simultaneously when the energy levels and T1 values between each organic material layer, as well as the material's inherent properties (mobility, interfacial properties, etc.) are optimally combined.

In addition, in order to solve the problem of light emission in the hole transport layer in recent organic light emitting devices, an emitting-auxiliary layer must exist between the hole transport layer and the emitting layer, and it is time to develop different emitting auxiliary layers for each emitting layer (R, G, B).

Typically, electrons are transferred from the electron transport layer to the emitting layer, and holes are transferred from the hole transport layer to the emitting layer, and excitons are generated through recombination.

However, since the material used in the hole transport layer must have a low HOMO value, most of them have a low T1 value, which causes excitons generated in the emitting layer to move to the hole transport layer, resulting in charge unbalance within the emitting layer and causing light emission at the interface of the hole transport layer.

When light is emitted at the interface of the hole transport layer, the color purity and efficiency of the organic electronic element are reduced, and its lifespan is shortened. Therefore, there is an urgent need to develop an emitting auxiliary layer with a high T1 value and a HOMO energy level between the HOMO energy level of the hole transport layer and the HOMO energy level of the emitting layer.

Meanwhile, it is necessary to develop a hole injection layer material that has stable properties, i.e., a high glass transition temperature, against Joule heating generated during device operation while delaying the penetration and diffusion of metal oxides from the anode electrode (ITO), which is one of the causes of shortened lifespan of organic electronic elements, into the organic layer. The low glass transition temperature of the hole transport layer material has been reported to significantly impact device lifespan by reducing the uniformity of the thin film surface during device operation. Furthermore, OLED devices are primarily formed through deposition, and there is a pressing need for materials that can withstand long-term deposition, i.e., materials with strong heat resistance.

In other words, in order to fully demonstrate the excellent characteristics of organic electronic elements, the materials forming the organic layers within the devices, such as hole injection materials, hole transport materials, emitting materials, electron transport materials, electron injection materials, and emitting-auxiliary layer materials, must first be supported by stable and efficient materials. However, the development of stable and efficient organic layer materials for organic electronic elements has not yet been sufficiently accomplished. Therefore, the development of new materials continues to be required, and in particular, the development of materials for emitting-auxiliary layers is urgently required.

### BRIEF DESCRIPTION OF THE INVENTION

### Summary

In order to solve the problems of the above-described background technology, the present invention has discovered a compound having a novel structure, and has also discovered that when this compound is applied to an organic electronic element, the luminous efficiency, stability, and lifespan of the element can be greatly improved.

Accordingly, the present invention aims to provide a novel compound, an organic electronic element using the same, and an electronic device thereof.

### Technical Solution

The present invention provides a compound represented by Formula 1.

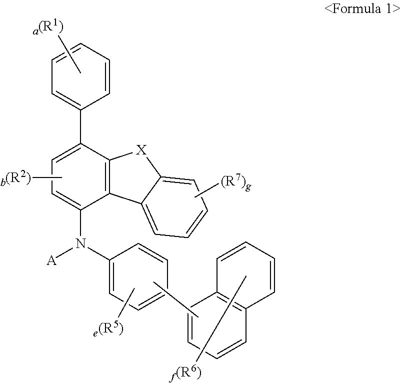

In another aspect, the present invention provides an organic electronic element comprising a compound represented by Formula 1 and an electronic device thereof.

### Effects of the Invention

By using the compound according to the present invention, it is possible to achieve a high luminous efficiency, a low driving voltage, and a high heat resistance of the element, and can greatly improve the color purity and lifespan of the element.

### BRIEF DESCRIPTION OF THE DRAWINGS

FIG. 1 to FIG. 3 illustrate an example of an organic electronic element according to the present invention.

FIG. 4 shows a Formula according to one aspect of the present invention.

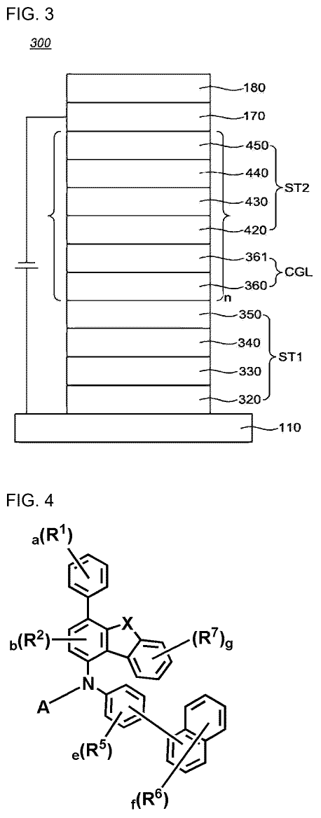

### DESCRIPTION OF THE NUMERALS IN THE DRAWINGS

| | |
|---|---|
| 100, 200, 300: organic electronic | 110: the first electrode |
| element | |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine (F), bromine (Br), chlorine (CI), or iodine (I).

Unless otherwise stated, the term “alkyl” or “alkyl group”, as used herein, has a single bond of 1 to 60 carbon atoms, 1 to 30 carbon atoms, 1 to 25 carbon atoms, 1 to 18 carbon atoms, or 1 to 12 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

More specifically it may be a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.

Unless otherwise stated, the term “alkenyl” or “alkynyl”, as used herein, has double or triple bonds of 2 to 60 carbon atoms, 2 to 30 carbon atoms, 2 to 25 carbon atoms, 2 to 18 carbon atoms, 2 to 12 carbon atoms, but is not limited thereto, and includes a linear or a branched chain group.

Unless otherwise stated, the term “cycloalkyl”, as used herein, means alkyl forming a ring having 3 to 60 carbon atoms, 3 to 30 carbon atoms, 3 to 25 carbon atoms, 3 to 18 carbon atoms, 3 to 12 carbon atoms, but is not limited thereto.

Unless otherwise stated, the term “alkoxyl group”, “alkoxy group” or “alkyloxy group”, as used herein, means an alkyl group bonded to oxygen radical, but is not limited thereto, and has 1 to 60 carbon atoms, 1 to 30 carbon atoms, 1 to 25 carbon atoms, 1 to 18 carbon atoms, or 1 to 12 carbon atoms.

Unless otherwise stated, the term “aryloxyl group” or “aryloxy group”, as used herein, means an aryl group bonded to oxygen radical, but is not limited thereto, and has 6 to 60 carbon atoms, 6 to 30 carbon atoms, 6 to 25 carbon atoms, 6 to 18 carbon atoms, or 6 to 12 carbon atoms.

Unless otherwise specified, the terms “aryl group” and “arylene group” used in the present invention have 6 to 60 carbon atoms, 6 to 30 carbon atoms, 6 to 25 carbon atoms, 6 to 18 carbon atoms, or 6 to 12 carbon atoms, respectively, but are not limited thereto.

In the present invention, an aryl group or arylene group refers to an aromatic group of a single ring or multiple rings, and comprises an aromatic ring formed by the bonding or reaction of adjacent substituents.

For example, the aryl group may be phenyl, biphenyl, naphthyl, terphenyl, phenanthrene or a combination thereof, particularly phenyl, naphthyl, phenanthrene or a combination thereof, and may also include a fluorene group or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, and has 2 to 60 carbon atoms, 2 to 30 carbon atoms, 2 to 25 carbon atoms, 2 to 18 carbon atoms, 2 to 12 carbon atoms, and comprises any one of a single ring or multiple ring, and may include heteroaliphatic ring and heteroaromatic ring. Also, the heterocyclic group may also be formed in conjunction with an adjacent group.

Unless otherwise stated, the term “heteroatom”, as used herein, represents at least one of N, O, S, P, or Si. Specifically, these may include pyridine, pyrimidine, triazine, indole, phenyl-indole, quinazoline, benzoquinazoline, quinoxaline, benzoquinoxaline, benzofuran, naphthobenzofuran, dibenzofuran, dynapthofuran, phenantrobenzofuran, thiophene, benzothiophene, dibenzothiophene, naphthobenzothiophene, dynapthobenzothiophene, phenantrobenzothiophene, carbazole, phenyl-carbazole, benzocarbazole, phenyl-benzocarbazole, naphthyl-benzocarbazole, dibenzocarbazole, indolocarbazole, benzothiophenopyrimidine, benzofuranopyrimidine, benzothiophenopyrazine, benzofuropyrazine, dibenzosilole, benzoxazole, naphthoxazole, dibenzothienoxazole, dibenzofurobenzoxazole, spiro[fluorene-9,9′-xanthene], etc., but are not limited to,

Additionally, “heterocyclic group” means a single ring, ring aggregate, fused multiple ring system, spiro compound, etc. containing a heteroatom. Also, compounds containing heteroatom groups such as SO₂, P═O, etc. instead of carbon forming a ring, such as the compounds below, can also be included in the heterocyclic group. For example, a “heterocyclic group” includes the following compound.

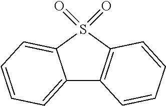 [SMILES: O=S1(=O)c2ccccc2-c2ccccc21]

The term “aliphatic ring group” used in the present invention refers to cyclic hydrocarbons excluding aromatic hydrocarbons, and includes single rings, ring aggregates, fused multiple ring systems, spiro compounds, etc., and means a ring having 3 to 60 carbon atoms, 3 to 30 carbon atoms, 3 to 25 carbon atoms, 3 to 18 carbon atoms, 3 to 12 carbon atoms, but is not limited thereto. For example, even when benzene, an aromatic ring, and cyclohexane, a non-aromatic ring, are fused, it is an aliphatic ring.

Unless otherwise stated, the term “fluorenyl group”, “fluorenylene group” or “fluorentriyl group” as used herein, means a monovalent, divalent or trivalent functional group, in which R, R′ and R″ are all hydrogen in the following structures, and the term “substituted fluorenyl group”, “substituted fluorenylene group” or “substituted fluorentriyl group” means that at least one of the substituents R, R′ and R″ is a substituent other than hydrogen, and include those in which R and R′ are bonded to each other to form a spiro compound together with the carbon to which they are bonded. In this specification, fluorenyl group, fluorenylene group, and fluorenetriyl group may all be referred to as fluorene groups, regardless of valence. For example, substituted fluorenyl groups may be dimethyl-fluorenyl groups, diphenylfluorenyl groups, or spirofluorenyl groups.

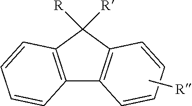

The term “spiro compound” used in the present invention has a ‘spiro union’, and a spiro union means a connection formed by 2 rings sharing only one atom. At this time, the atom shared between the 2 rings is called a ‘spiro atom’, and depending on the number of spiro atoms contained in a compound, they are called ‘monospiro-’, ‘dispiro-’, and ‘trispiro-’ compounds, respectively.

Unless otherwise stated, the term “aliphatic” as used herein means an aliphatic hydrocarbon having 1 to 60 carbon atoms, 1 to 30 carbon atoms, 1 to 25 carbon atoms, 1 to 18 carbon atoms or 1 to 12 carbon atoms, and “aliphatic ring” means an aliphatic hydrocarbon ring having 3 to 60 carbon atoms, 3 to 30 carbon atoms, 3 to 25 carbon atoms, 3 to 18 carbon atoms or 3 to 12 carbon atoms.

Unless otherwise stated, the term “ring”, as used herein, means an aliphatic ring having 3 to 60 carbon atoms, 3 to 30 carbon atoms, 3 to 25 carbon atoms, 3 to 18 carbon atoms or 3 to 12 carbon atoms; or an aromatic ring having 6 to 60 carbon atoms, 6 to 30 carbon atoms, 6 to 25 carbon atoms, 6 to 18 carbon atoms, or 6 to 12 carbon atoms; or a heterocyclic having 2 to 60 carbon atoms, 2 to 30 carbon atoms, 2 to 25 carbon atoms, 2 to 18 carbon atoms, 2 to 12 carbon atoms, or a fused ring formed by the combination thereof, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

In this specification, the ‘group name’ corresponding to the aryl group, arylene group, heterocyclic group, etc., as examples of each symbol and its substituent, may be written as the ‘name of the group reflecting the valence’, but is written as the ‘parent compound name’. For example, in the case of ‘phenanthrene’, a type of aryl group, the name of the group may be written by distinguishing the valence, such as the monovalent ‘group’ is ‘phenanthryl’ and the divalent group is ‘phenanthrylene’, but may be written as ‘phenanthrene’, which is the name of the parent compound, regardless of the valence. Similarly, in the case of pyrimidine, it can be written as ‘pyrimidine’ regardless of the valence, or it can be written as the ‘name of the group’ of the valence, such as pyrimidineyl group in the case of monovalent group, pyrimidineylene in the case of divalent group, etc. Additionally, in this specification, when describing compound names or substituent names, numbers or alphabets indicating positions may be omitted. For example, pyrido[4,3-d]pyrimidine to pyridopyrimidine, benzofuro[2,3-d]pyrimidine to benzofuropyrimidine, 9,9-dimethyl-9H-fluorene can be described as dimethylfluorene, etc. Therefore, both benzo[g]quinoxaline and benzo[f]quinoxaline can be described as benzoquinoxaline.

Also, unless there is an explicit explanation, the formula used in the present invention is the same as the definition of the substituent by the exponent definition of the following formula.

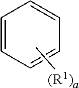

Here, when a is an integer of 0, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

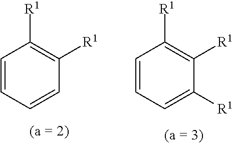

Unless otherwise expressly stated, the terms “ortho”, “meta”, and “para” used in the present invention refer to the substitution positions of all substituents, and the ortho position refers to a compound in which the position of the substituent is immediately adjacent, for example, when benzene is used, it means 1 or 2 position, and the meta position is the next substitution position of the neighbor substitution position, when benzene as an example stands for 1 or 3 position, and the para position is the next substitution position of the meta position, which means 1 and 4 position when benzene is taken as an example. A more detailed example of the substitution position is as follows, and it can be confirmed that the ortho-, and meta-position are substituted by non-linear type and para-positions are substituted by linear type.

### [Example of Ortho-Position]

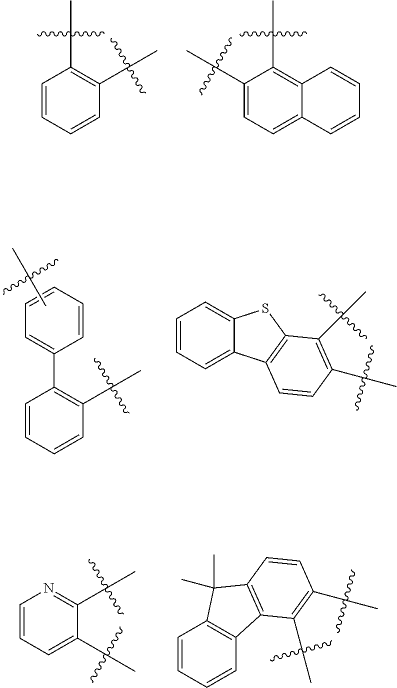

### [Example of Meta-Position]

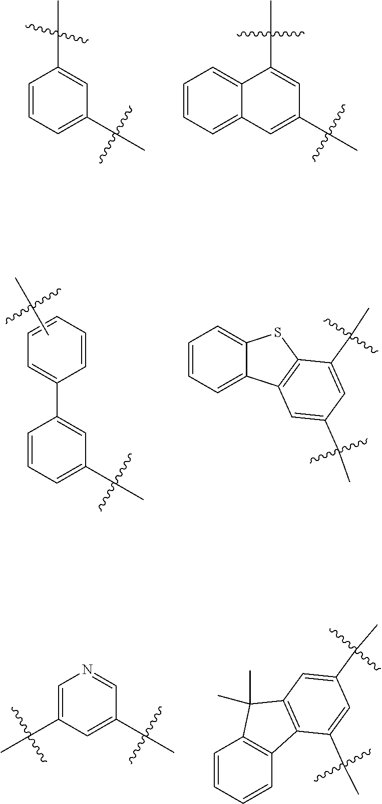

### [Example of Para-Position]

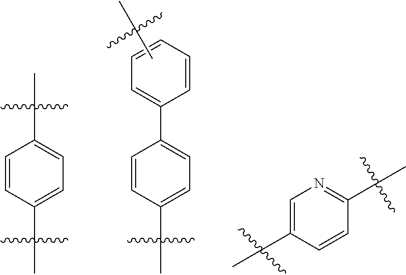

Hereinafter, a compound according to one aspect of the present invention and an organic electronic element comprising the same will be described.

The present invention provides a compound represented by Formula 1:

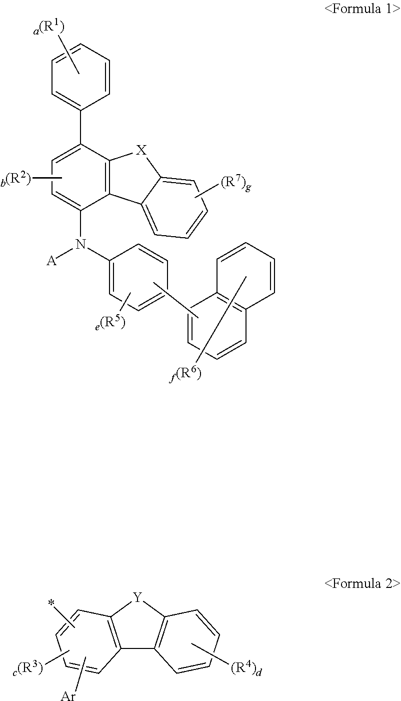

wherein:
X and Y are independently O or S,
R¹ is independently the same or different from each other and are independently selected from the group consisting of hydrogen; deuterium; a C₁-C₅₀ alkyl group; a C₃-C₆₀ aliphatic ring; a fluorenyl group; or a C₆-C₆₀ aryl group; and a plurality of adjacent groups thereof may be bonded to each other to form a ring,
R², R³, R⁴, R⁵, R⁶ and R⁷ are independently the same or different from each other and are independently selected from the group consisting of hydrogen; deuterium; a C₆-C₆₀ aryl group; a fluorenyl group; a C₂-C₆₀ heterocyclic group including at least one heteroatom of O, N, S, Si and P; a C₃-C₆₀ aliphatic ring; and a C₁-C₅₀ alkyl group; and a plurality of adjacent groups thereof may be bonded to each other to form a ring,
a is an integer of 0 to 5, b and c are an integer of 0 to 2, d, e and g are independently an integer of 0 to 4, f is an integer of 0 to 7,
* indicates a position to be bonded, and A is a substituent represented by Formula 2,
Ar is a C₁-C₅₀ alkyl group; a C₃-C₆₀ aliphatic ring; a fluorenyl group; or a C₆-C₆₀ aryl group;
where R¹, R², R³, R⁴, R⁵, R⁶ and R⁷ are an aryl group, preferably it is a C₆-C₃₀ aryl group, more preferably an C₆-C₂₅ aryl group, an C₆-C₁₈ aryl group or an C₆-C₁₂ aryl group, such as phenyl, biphenyl, terphenyl, naphthalene, phenanthrene, etc.,
where R¹, R², R³, R⁴, R⁵, R⁶ and R⁷ are a heterocyclic group, it is preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.,
where R¹, R², R³, R⁴, R⁵, R⁶ and R⁷ are an alkyl group, it is preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.,
where R¹, R², R³, R⁴, R⁵, R⁶ and R⁷ are an aliphatic ring group, it is preferably a C₃-C₃₀ aliphatic ring group, more preferably a C₅-C₂₅ aliphatic ring group, a C₃-C₁₈ aliphatic ring group, and a C₃-C₁₂ aliphatic ring group, and specifically, cyclobutane, cyclopentane, cyclohexane, bicycloheptane, adamantyl, etc.,
where Ar is an aryl group, it is preferably an C₆-C₃₀ aryl group, more preferably an C₆-C₂₅ aryl group, an C₆-C₁₈ aryl group or an C₆-C₁₂ aryl group, such as phenyl, biphenyl, terphenyl, naphthalene, phenanthrene, etc.,
where Ar is an alkyl group, it is preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.
where Ar is an aliphatic ring group, it is preferably a C₃-C₃₀ aliphatic ring group, more preferably a C₃-C₂₅ aliphatic ring group, a C₅-C₁₈ aliphatic ring group, and a C₃-C₁₂ aliphatic ring group, and specifically, cyclobutane, cyclopentane, cyclohexane, cyclohexacene, bicycloheptane, adamantyl, etc.,
wherein Formula 2-A is excluded from Formula 2:

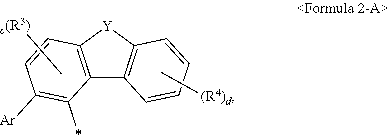

wherein the aryl group, heterocyclic group, fluorenyl group, and alkyl group may be substituted with one or more substituents selected from the group consisting of cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic ring; a C₇-C₂₀ arylalkyl group; a C₈-C₂₀ arylalkenyl group; and a C₇-C₂₀ alkylaryl group, and the hydrogen of these substituents may be further substituted with one or more deuterium, and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the present invention provides a compound in which Formula 1 is represented by any one of the following Formulas 1-1 to 1-6. In the present invention, Formula 1 may preferably be selected from any one of Formulas 1-1 to 1-6, more preferably from any one of Formulas 1-1 to 1-3, and even more preferably from Formula 1-1.

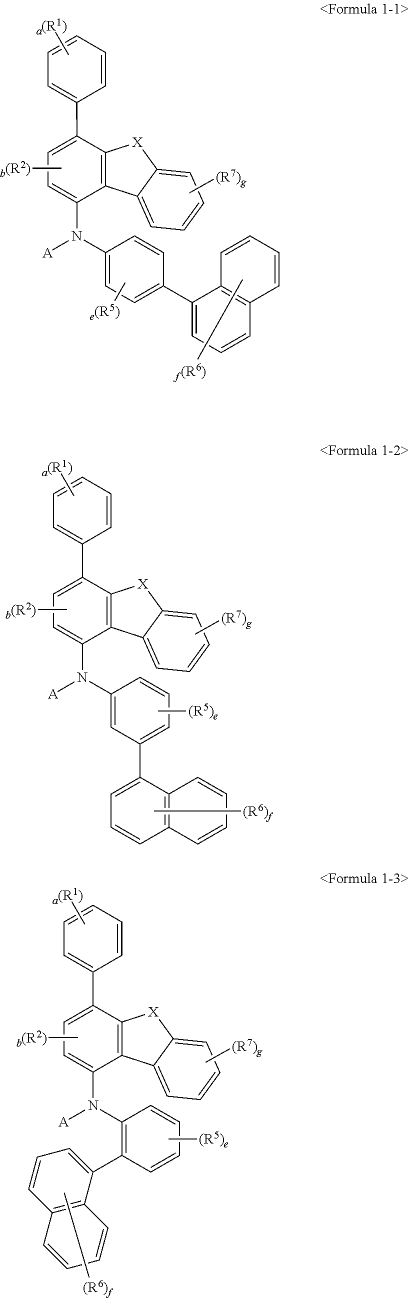

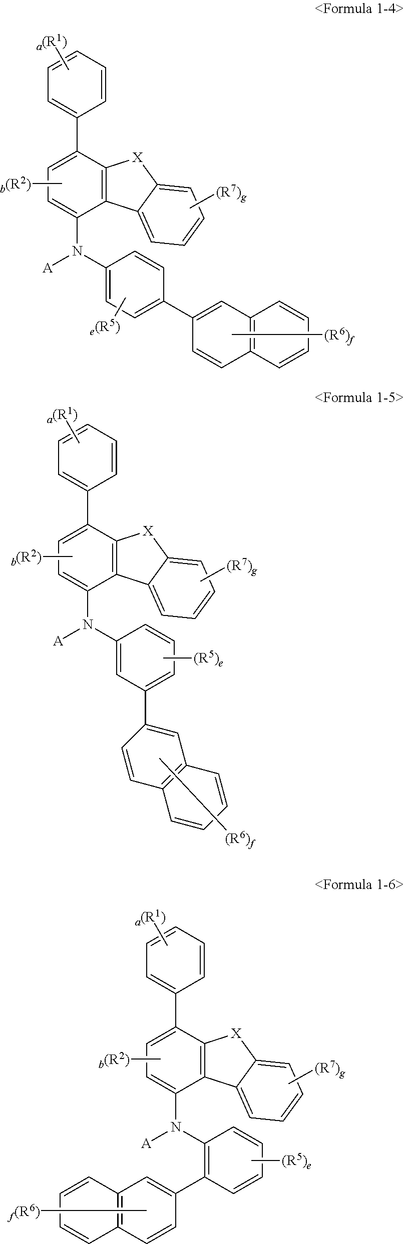

wherein, X, A, R¹, R², R⁵, R⁶, R⁷, a, b, e, f and g are the same as defined above.

Also, Formula 2 is represented by any one of Formulas 2-1 to 2-4.

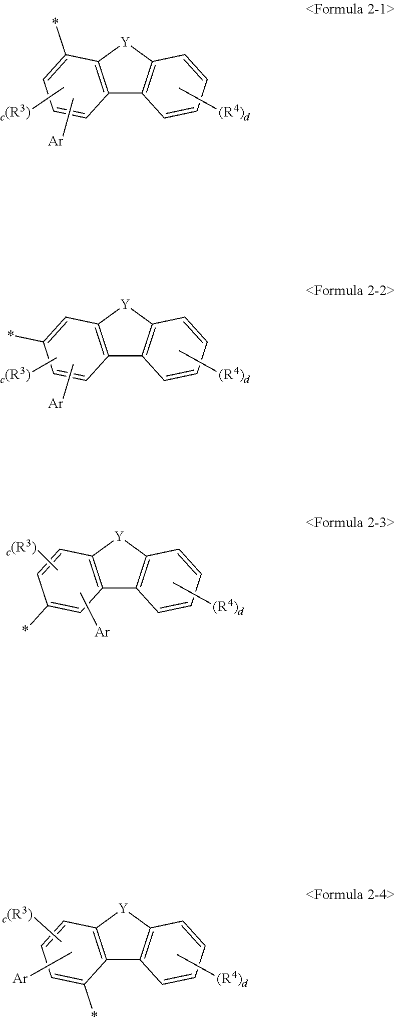

wherein Ar, Y, R³, R⁴, c, d and * are the same as defined above.

In the present invention, Formula 2 may preferably be selected from any one of the following Formulas 2-1A to 2-4B, and more preferably from any one of the following Formulas 2-4A and 2-4B.

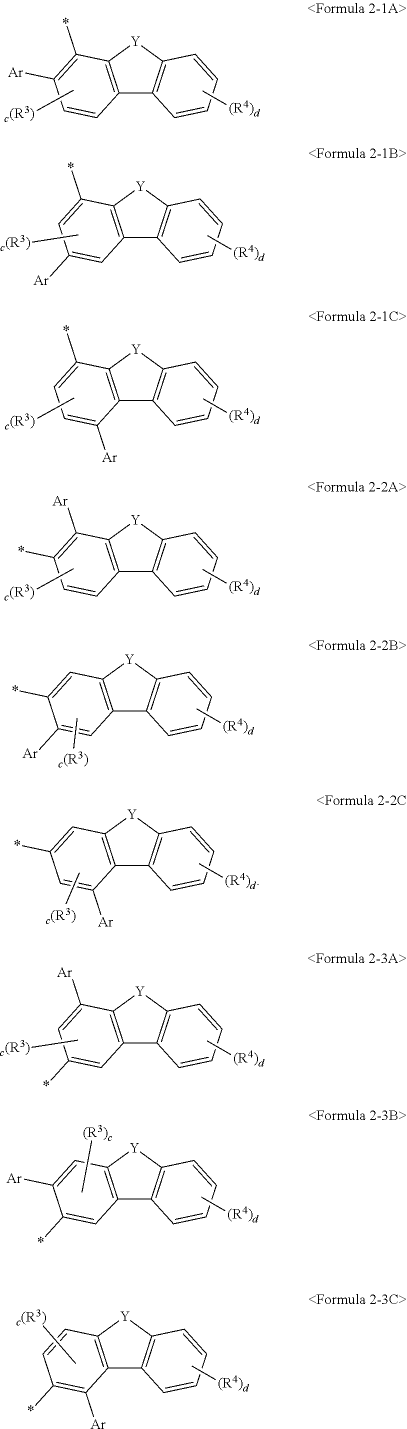

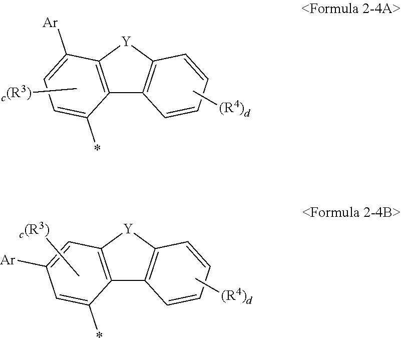

As another example, the Ar is represented by any one of the following Formulas Ar-1 to Ar-15.

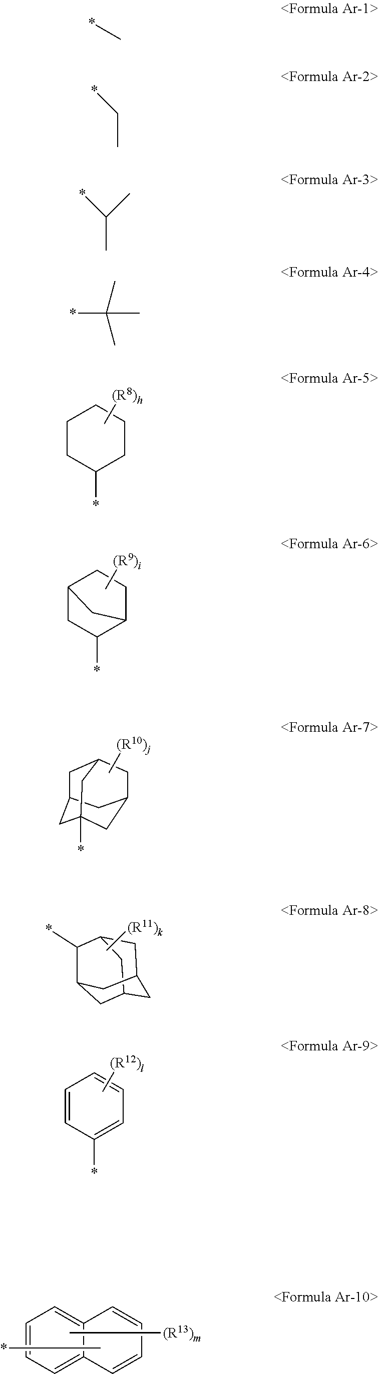

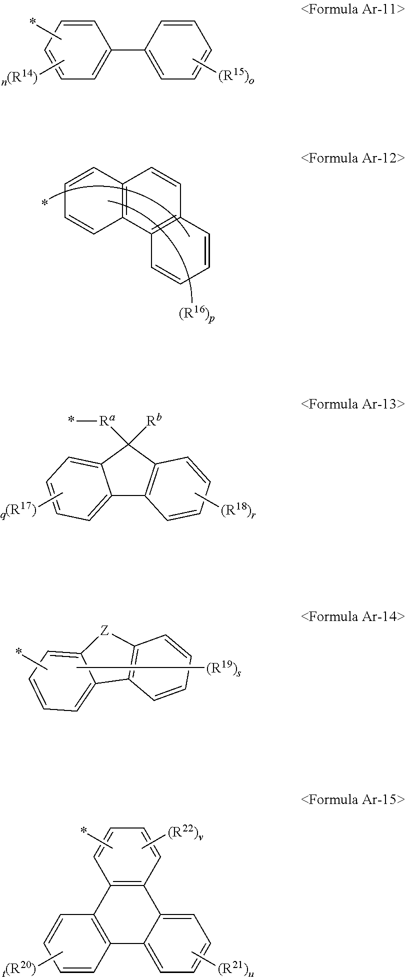

wherein:
R⁸, R⁹, R¹⁰, R¹¹, R¹², R¹³, R¹⁴, R¹⁵, R¹⁶, R¹⁷, R¹⁸, R¹⁹, R²⁰, R²¹ and R²² are independently the same or different from each other and are independently selected from the group consisting of hydrogen; deuterium; a C₆-C₄₀ aryl group; a fluorenyl group; a C₂-C₄₀ heterocyclic group including at least one heteroatom of O, N, S, Si and P; a C₃-C₄₀ aliphatic ring; a C₁-C₂₀ alkyl group; a C₂-C₂₀ alkenyl group; a C₂-C₂₀ alkynyl group; a C₁-C₃₀ alkoxyl group; and a C₆-C₃₀ an aryloxy group; and a plurality of adjacent groups thereof may be bonded to each other to form an aromatic ring, and R⁸, R⁹, R¹⁰ and R¹¹ may preferably be hydrogen or deuterium,
Z is CR^{c}R^{d},
R^{a}, R^{b}, R^{c}, R^{d} are independently the same or different from each other and are independently selected from the group consisting of hydrogen; deuterium; a C₆-C₂₀ aryl group; a C₆-C₂₀ aryl group substituted with deuterium; a fluorenyl group; a C₂-C₂₀ heterocyclic group; a C₅-C₂₀ cycloalkyl group; a C₁-C₂₀ alkyl group; and a C₂-C₂₀ alkenyl group; and a plurality of adjacent R^{a} and R^{b}, or a plurality of adjacent R^{c} and R^{d} may be bonded to each other to form an aromatic ring,
h, l and o are integers from 0 to 5, i is integer from 0 to 6, j, k and p are integers from 0 to 9, m is integer from 0 to 7, n, q, r, t and u are integers from 0 to 4, s is integer from 0 to 7 and v is integer from 0 to 3.

As another example, Formulas Ar-1 to Ar-4 may be further substituted with deuterium.

Specifically, the compounds represented by Formula 1 include, but are not limited to, the following compounds P-1 to P-125:

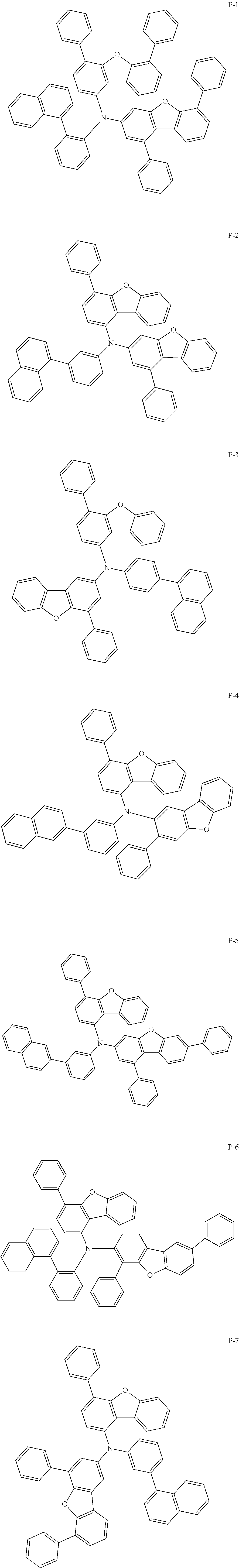

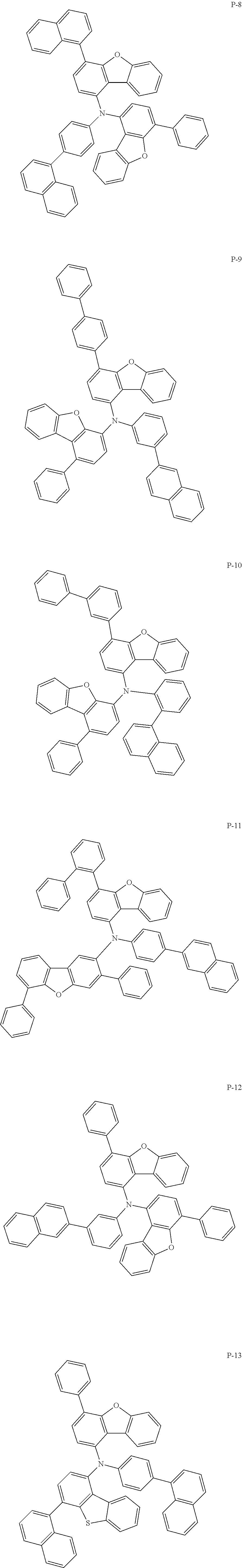

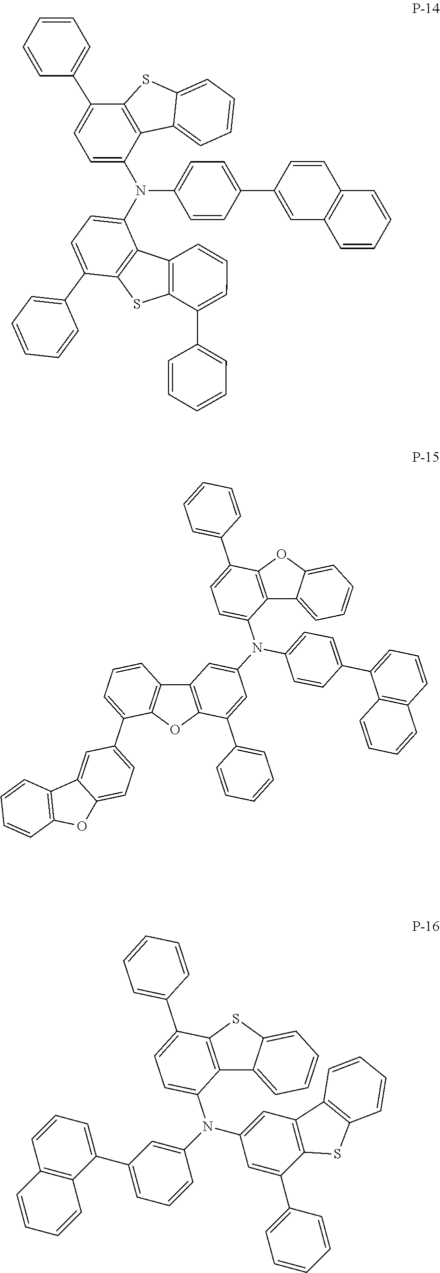 [SMILES: c1ccc(-c2cccc3c2sc2c(-c4ccccc4)ccc(N(c4ccc(-c5ccc6ccccc6c5)cc4)c4ccc(-c5ccccc5)c5sc6ccccc6c45)c23)cc1 ; c1ccc(-c2cc(N(c3ccc(-c4cccc5ccccc45)cc3)c3ccc(-c4ccccc4)c4oc5ccccc5c34)cc3c2oc2c(-c4ccc5oc6ccccc6c5c4)cccc23)cc1 ; c1ccc(-c2cc(N(c3cccc(-c4cccc5ccccc45)c3)c3ccc(-c4ccccc4)c4sc5ccccc5c34)cc3c2sc2ccccc23)cc1]

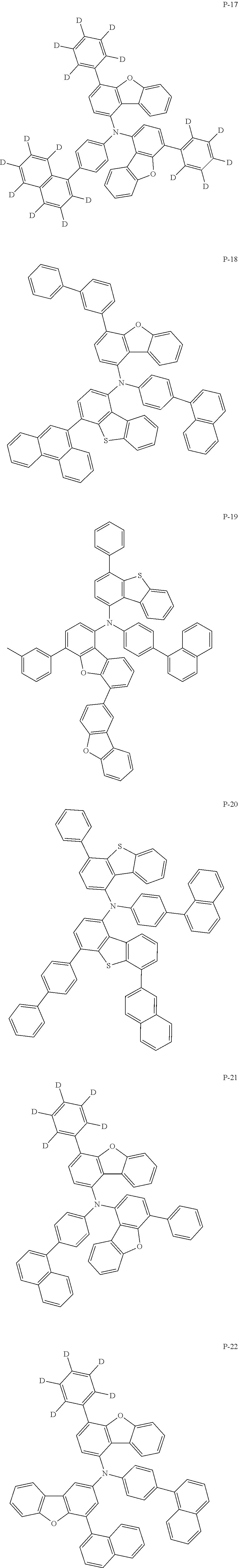

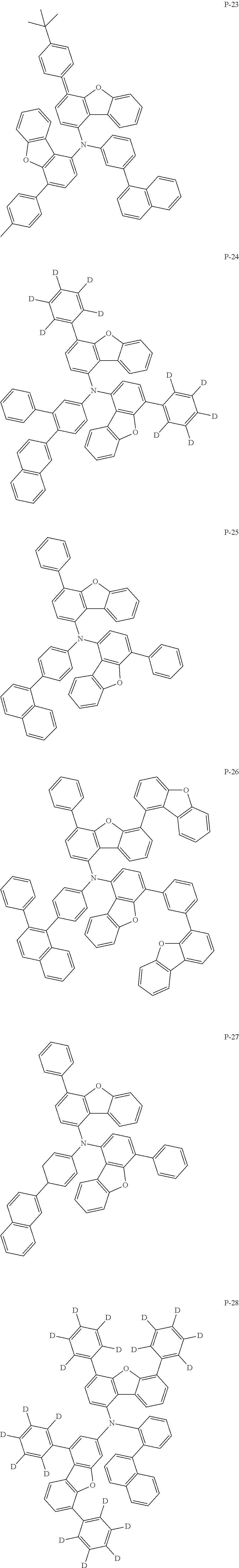

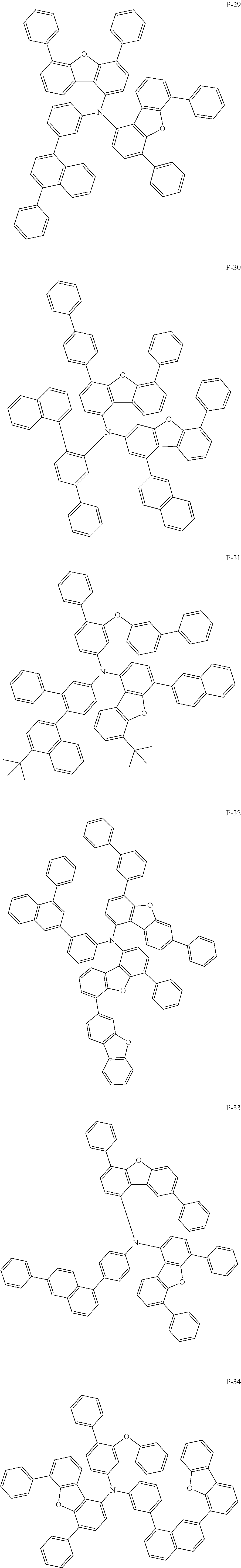

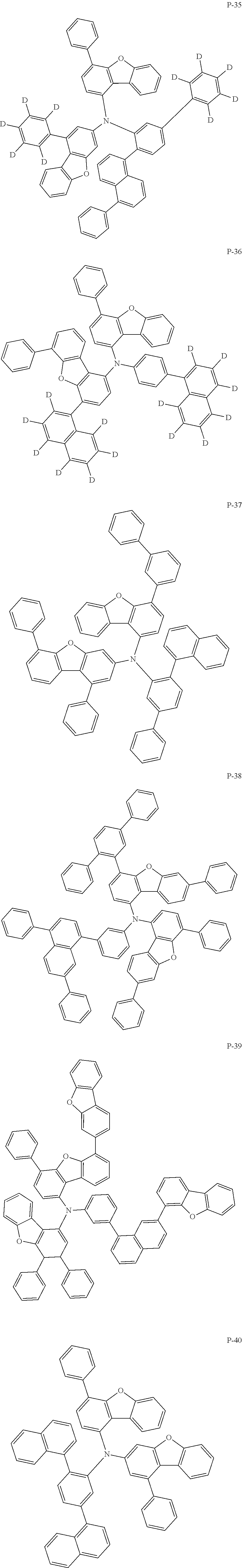

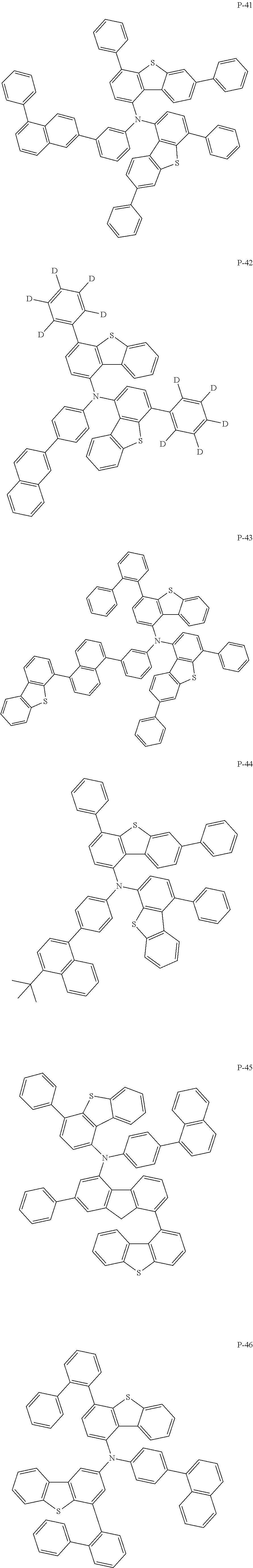

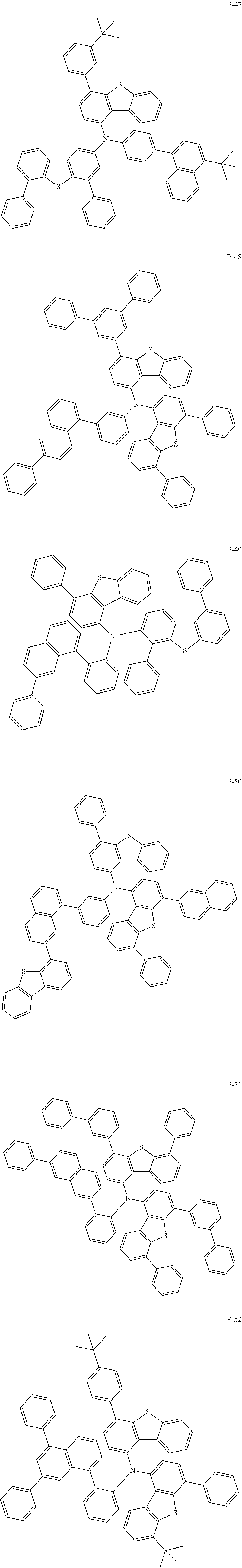

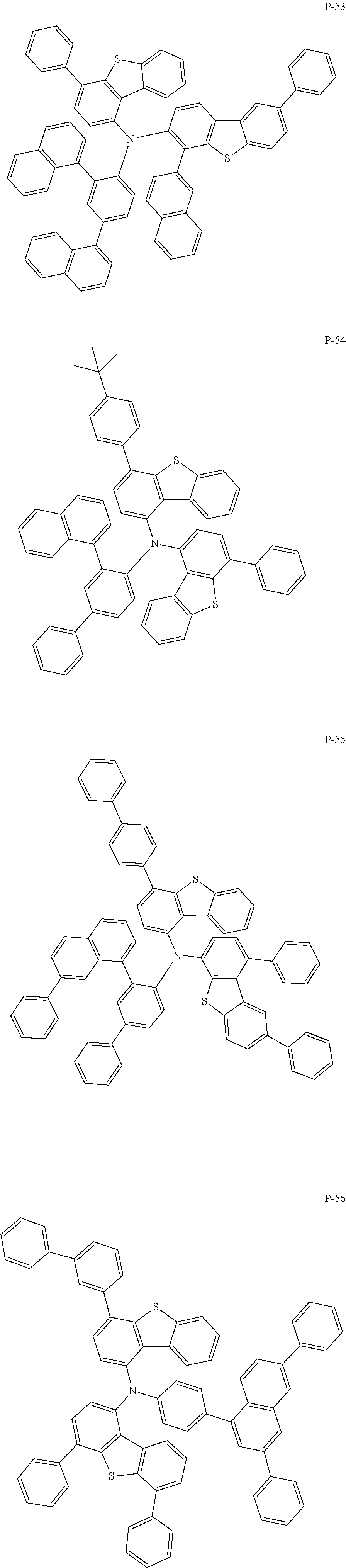

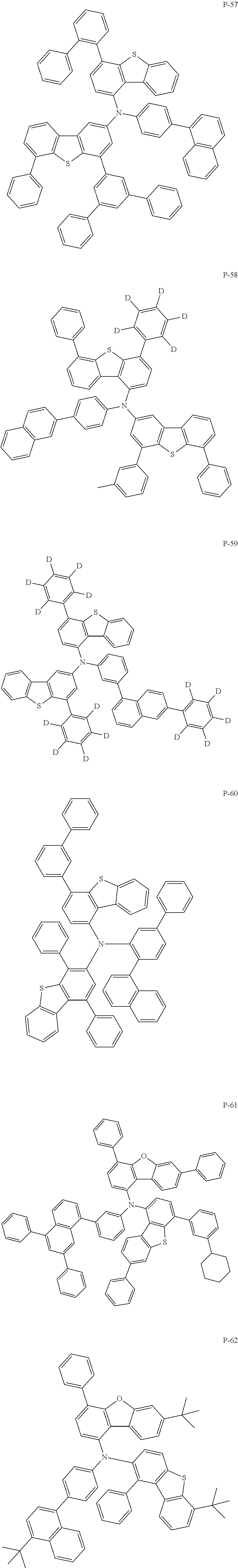

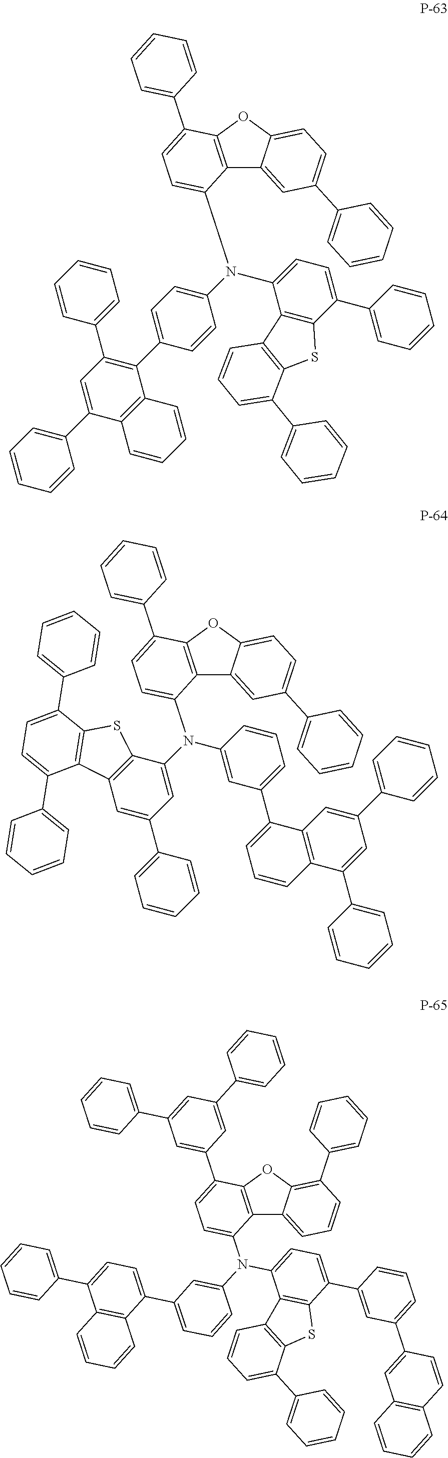 [SMILES: c1ccc(-c2ccc3oc4c(-c5ccccc5)ccc(N(c5ccc(-c6c(-c7ccccc7)cc(-c7ccccc7)c7ccccc67)cc5)c5ccc(-c6ccccc6)c6sc7c(-c8ccccc8)cccc7c56)c4c3c2)cc1 ; c1ccc(-c2cc(-c3ccccc3)c3cccc(-c4cccc(N(c5cc(-c6ccccc6)cc6c5sc5c(-c7ccccc7)ccc(-c7ccccc7)c56)c5ccc(-c6ccccc6)c6oc7ccc(-c8ccccc8)cc7c56)c4)c3c2)cc1 ; c1ccc(-c2cc(-c3ccccc3)cc(-c3ccc(N(c4cccc(-c5ccc(-c6ccccc6)c6ccccc56)c4)c4ccc(-c5cccc(-c6ccc7ccccc7c6)c5)c5sc6c(-c7ccccc7)cccc6c45)c4c3oc3c(-c5ccccc5)cccc34)c2)cc1]

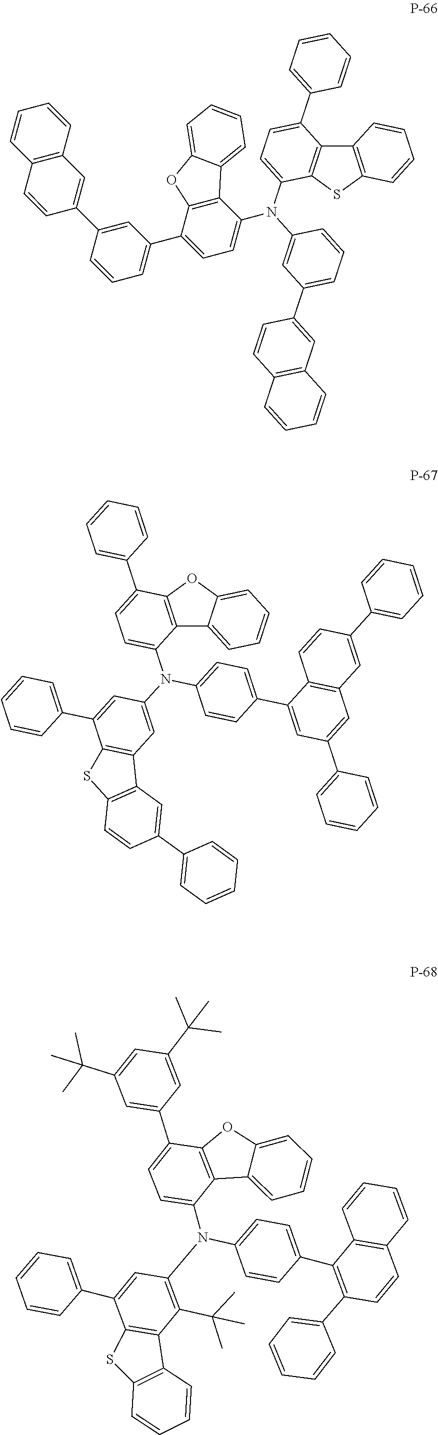 [SMILES: c1ccc(-c2ccc(N(c3cccc(-c4ccc5ccccc5c4)c3)c3ccc(-c4cccc(-c5ccc6ccccc6c5)c4)c4oc5ccccc5c34)c3sc4ccccc4c23)cc1 ; c1ccc(-c2ccc3c(-c4ccc(N(c5cc(-c6ccccc6)c6sc7ccc(-c8ccccc8)cc7c6c5)c5ccc(-c6ccccc6)c6oc7ccccc7c56)cc4)cc(-c4ccccc4)cc3c2)cc1 ; CC(C)(C)c1cc(-c2ccc(N(c3ccc(-c4c(-c5ccccc5)ccc5ccccc45)cc3)c3cc(-c4ccccc4)c4sc5ccccc5c4c3C(C)(C)C)c3c2oc2ccccc23)cc(C(C)(C)C)c1]

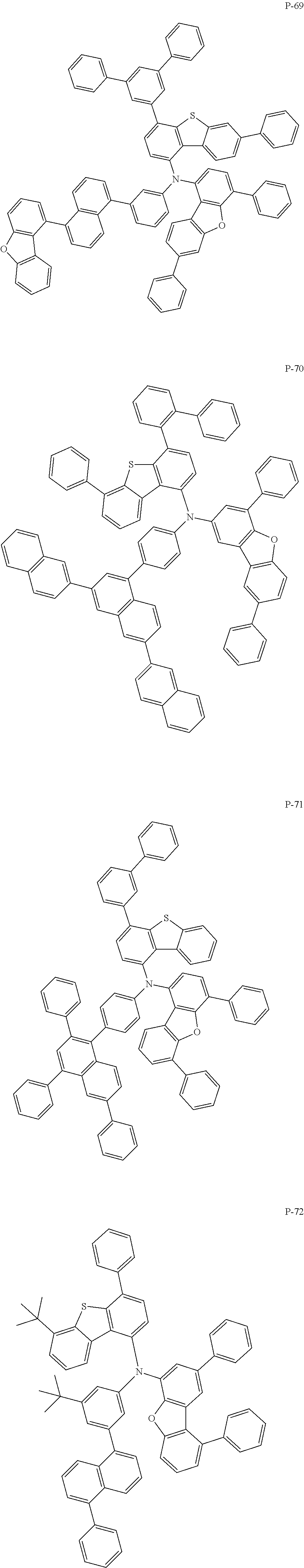

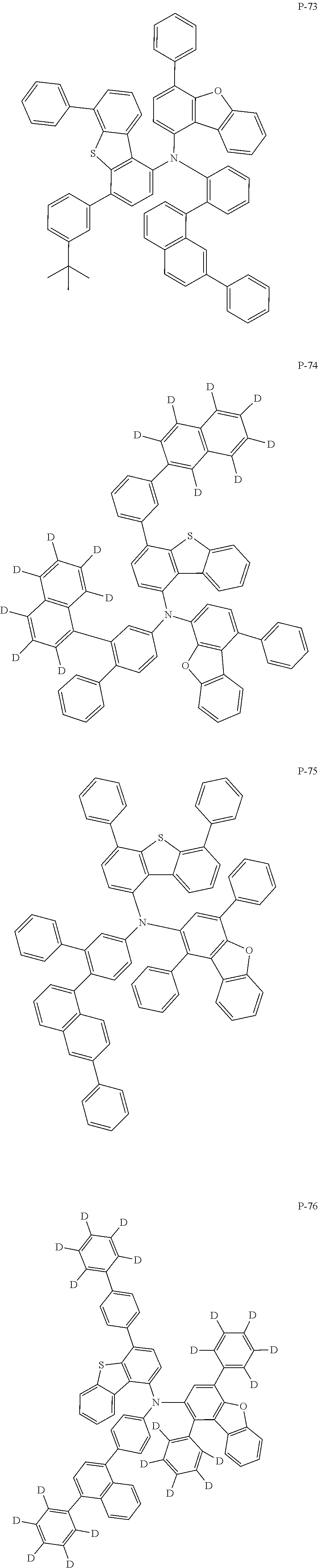

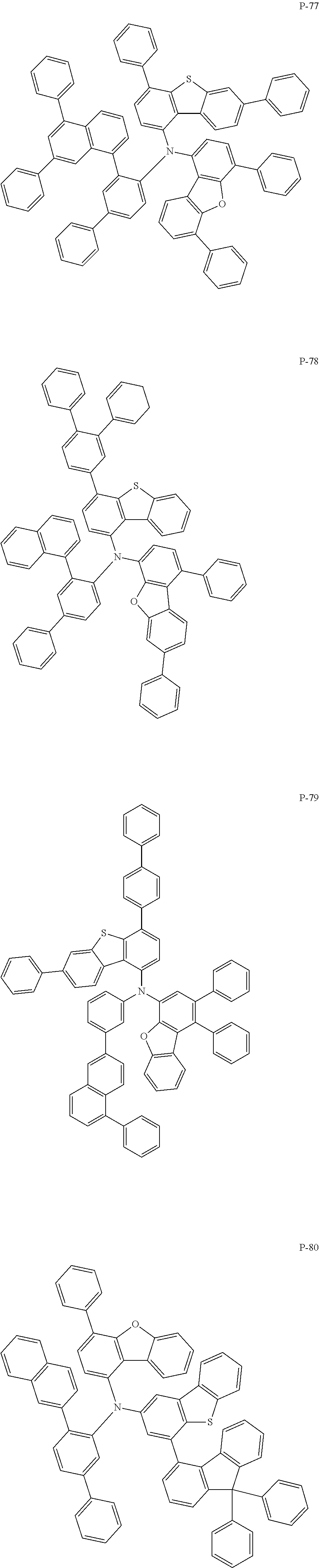

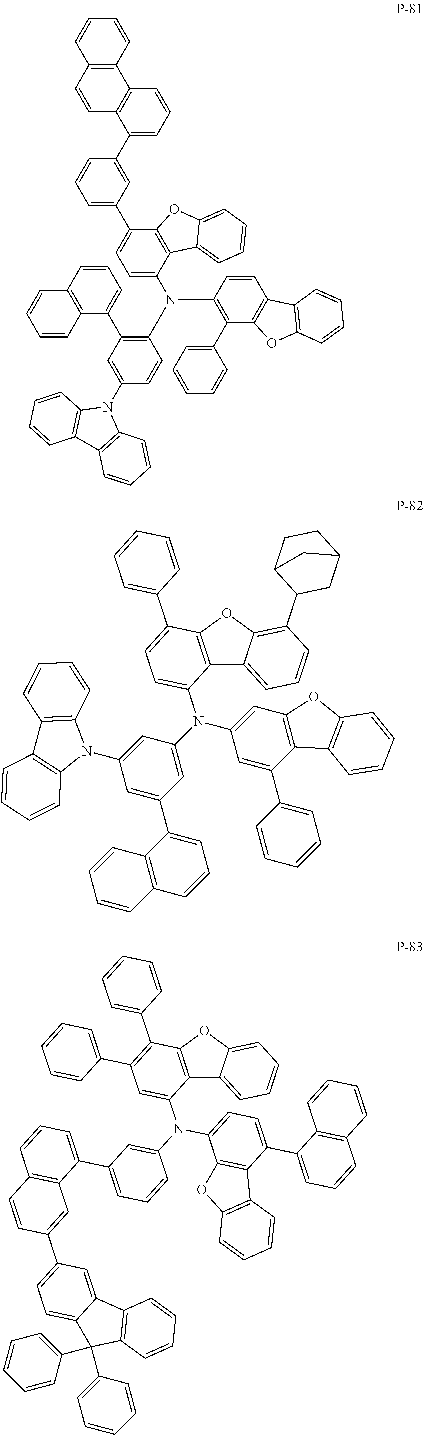 [SMILES: c1ccc(-c2c(N(c3ccc(-n4c5ccccc5c5ccccc54)cc3-c3cccc4ccccc34)c3ccc(-c4cccc(-c5cccc6c5ccc5ccccc56)c4)c4oc5ccccc5c34)ccc3c2oc2ccccc23)cc1 ; c1ccc(-c2ccc(N(c3cc(-c4cccc5ccccc45)cc(-n4c5ccccc5c5ccccc54)c3)c3cc(-c4ccccc4)c4c(c3)oc3ccccc34)c3c2oc2c(C4CC5CCC4C5)cccc23)cc1 ; c1ccc(-c2cc(N(c3cccc(-c4cccc5ccc(-c6ccc7c(c6)-c6ccccc6C7(c6ccccc6)c6ccccc6)cc45)c3)c3ccc(-c4cccc5ccccc45)c4c3oc3ccccc34)c3c(oc4ccccc43)c2-c2ccccc2)cc1]

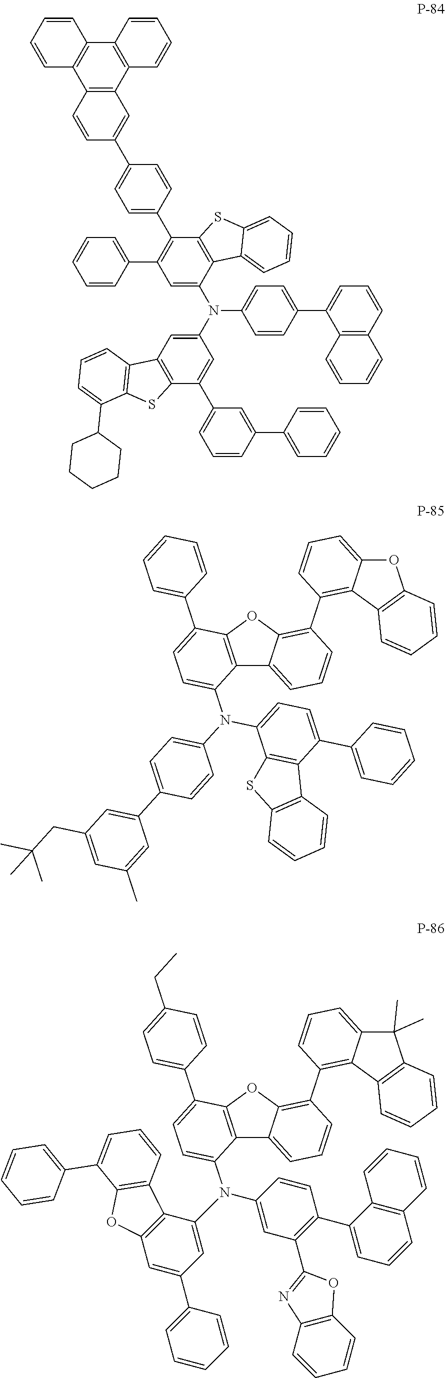 [SMILES: c1ccc(-c2cccc(-c3cc(N(c4ccc(-c5cccc6ccccc56)cc4)c4cc(-c5ccccc5)c(-c5ccc(-c6ccc7c8ccccc8c8ccccc8c7c6)cc5)c5sc6ccccc6c45)cc4c3sc3c(C5CCCCC5)cccc34)c2)cc1 ; Cc1cc(CC(C)(C)C)cc(-c2ccc(N(c3ccc(-c4ccccc4)c4c3sc3ccccc34)c3ccc(-c4ccccc4)c4oc5c(-c6cccc7oc8ccccc8c67)cccc5c34)cc2)c1 ; CCc1ccc(-c2ccc(N(c3ccc(-c4cccc5ccccc45)c(-c4nc5ccccc5o4)c3)c3cc(-c4ccccc4)cc4oc5c(-c6ccccc6)cccc5c34)c3c2oc2c(-c4cccc5c4-c4ccccc4C5(C)C)cccc23)cc1]

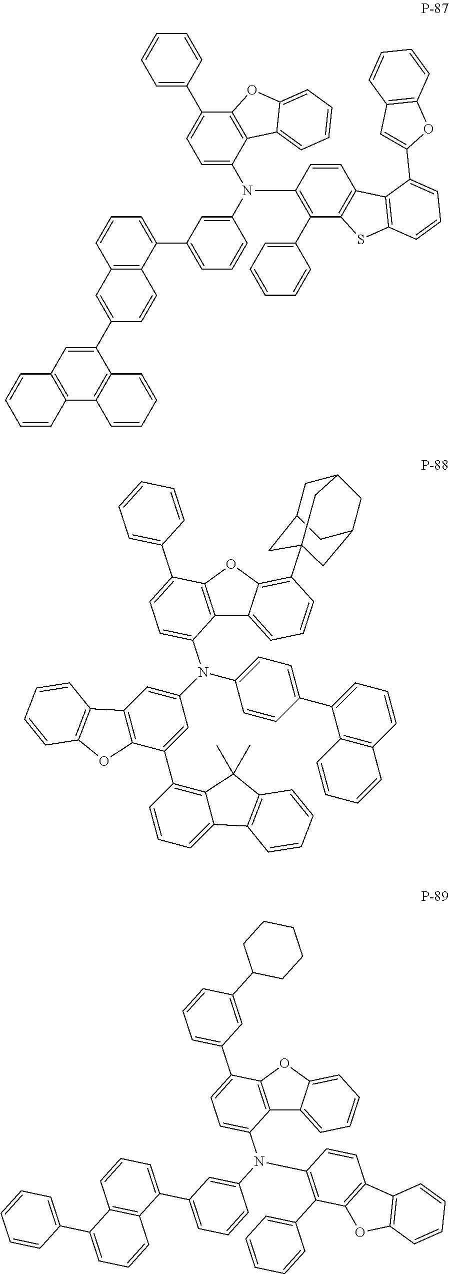 [SMILES: c1ccc(-c2ccc(N(c3cccc(-c4cccc5cc(-c6cc7ccccc7c7ccccc67)ccc45)c3)c3ccc4c(sc5cccc(-c6cc7ccccc7o6)c54)c3-c3ccccc3)c3c2oc2ccccc23)cc1 ; CC1(C)c2ccccc2-c2cccc(-c3cc(N(c4ccc(-c5cccc6ccccc56)cc4)c4ccc(-c5ccccc5)c5oc6c(C78CC9CC(CC(C9)C7)C8)cccc6c45)cc4c3oc3ccccc34)c21 ; c1ccc(-c2cccc3c(-c4cccc(N(c5ccc6c(oc7ccccc76)c5-c5ccccc5)c5ccc(-c6cccc(C7CCCCC7)c6)c6oc7ccccc7c56)c4)cccc23)cc1]

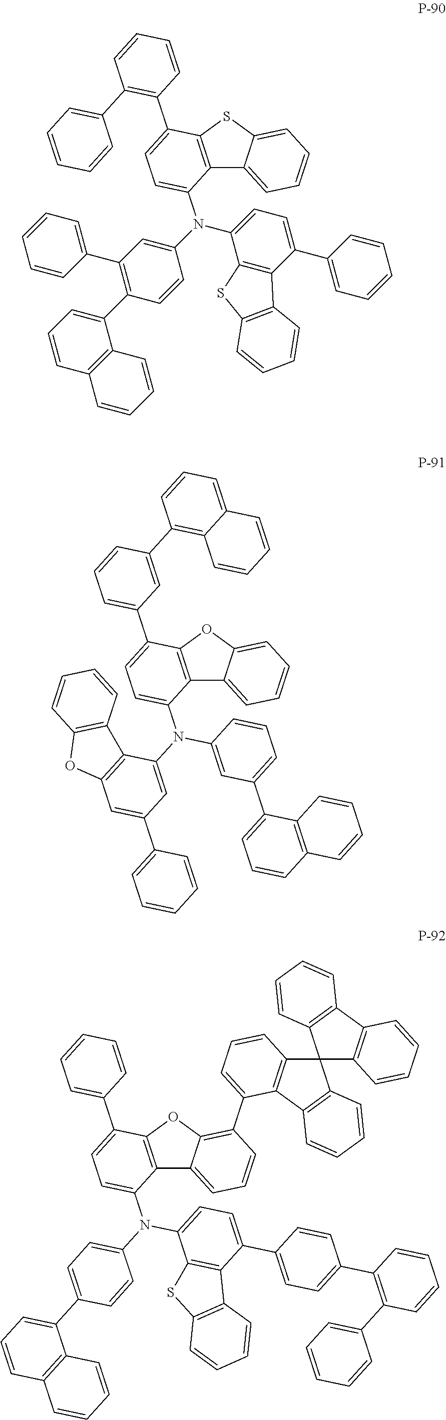 [SMILES: c1ccc(-c2cc(N(c3ccc(-c4ccccc4)c4c3sc3ccccc34)c3ccc(-c4ccccc4-c4ccccc4)c4sc5ccccc5c34)ccc2-c2cccc3ccccc23)cc1 ; c1ccc(-c2cc(N(c3cccc(-c4cccc5ccccc45)c3)c3ccc(-c4cccc(-c5cccc6ccccc56)c4)c4oc5ccccc5c34)c3c(c2)oc2ccccc23)cc1 ; c1ccc(-c2ccccc2-c2ccc(-c3ccc(N(c4ccc(-c5cccc6ccccc56)cc4)c4ccc(-c5ccccc5)c5oc6c(-c7cccc8c7-c7ccccc7C87c8ccccc8-c8ccccc87)cccc6c45)c4sc5ccccc5c34)cc2)cc1]

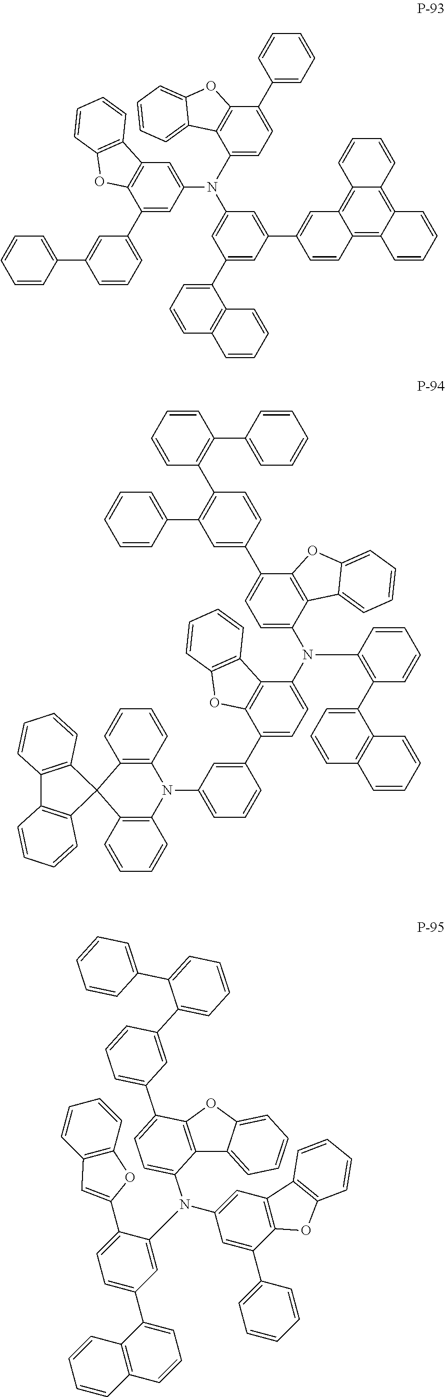 [SMILES: c1ccc(-c2cccc(-c3cc(N(c4cc(-c5ccc6c7ccccc7c7ccccc7c6c5)cc(-c5cccc6ccccc56)c4)c4ccc(-c5ccccc5)c5oc6ccccc6c45)cc4c3oc3ccccc34)c2)cc1 ; c1ccc(-c2ccccc2-c2ccc(-c3ccc(N(c4ccccc4-c4cccc5ccccc45)c4ccc(-c5cccc(N6c7ccccc7C7(c8ccccc8-c8ccccc87)c7ccccc76)c5)c5oc6ccccc6c45)c4c3oc3ccccc34)cc2-c2ccccc2)cc1 ; c1ccc(-c2ccccc2-c2cccc(-c3ccc(N(c4cc(-c5ccccc5)c5oc6ccccc6c5c4)c4cc(-c5cccc6ccccc56)ccc4-c4cc5ccccc5o4)c4c3oc3ccccc34)c2)cc1]

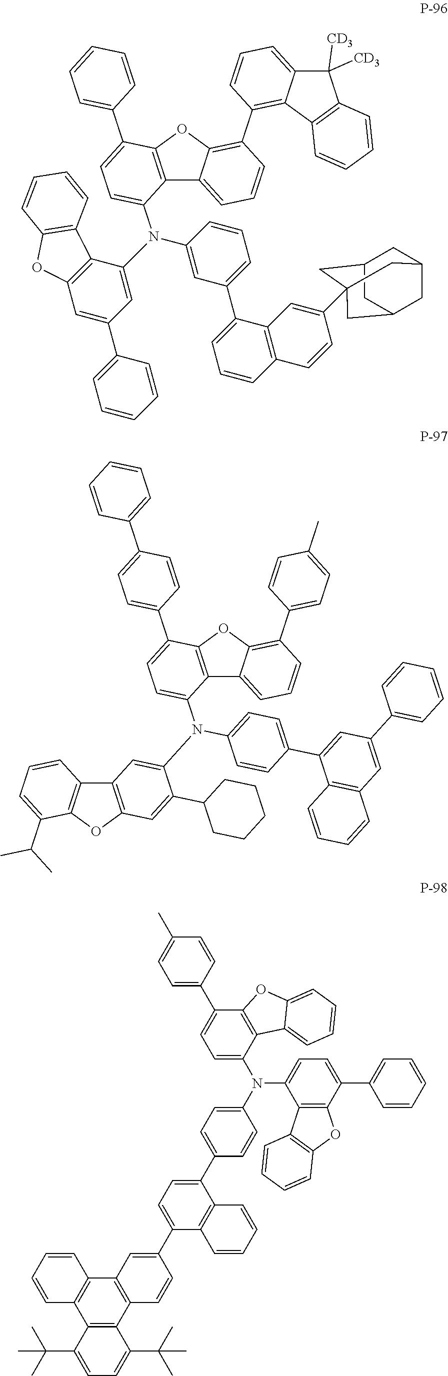 [SMILES: [2H]C([2H])([2H])C1(C([2H])([2H])[2H])c2ccccc2-c2c(-c3cccc4c3oc3c(-c5ccccc5)ccc(N(c5cccc(-c6cccc7ccc(C89CC%10CC(CC(C%10)C8)C9)cc67)c5)c5cc(-c6ccccc6)cc6oc7ccccc7c56)c34)cccc21 ; Cc1ccc(-c2cccc3c2oc2c(-c4ccc(-c5ccccc5)cc4)ccc(N(c4ccc(-c5cc(-c6ccccc6)cc6ccccc56)cc4)c4cc5c(cc4C4CCCCC4)oc4c(C(C)C)cccc45)c23)cc1 ; Cc1ccc(-c2ccc(N(c3ccc(-c4ccc(-c5ccc6c(c5)c5ccccc5c5c(C(C)(C)C)ccc(C(C)(C)C)c65)c5ccccc45)cc3)c3ccc(-c4ccccc4)c4oc5ccccc5c34)c3c2oc2ccccc23)cc1]

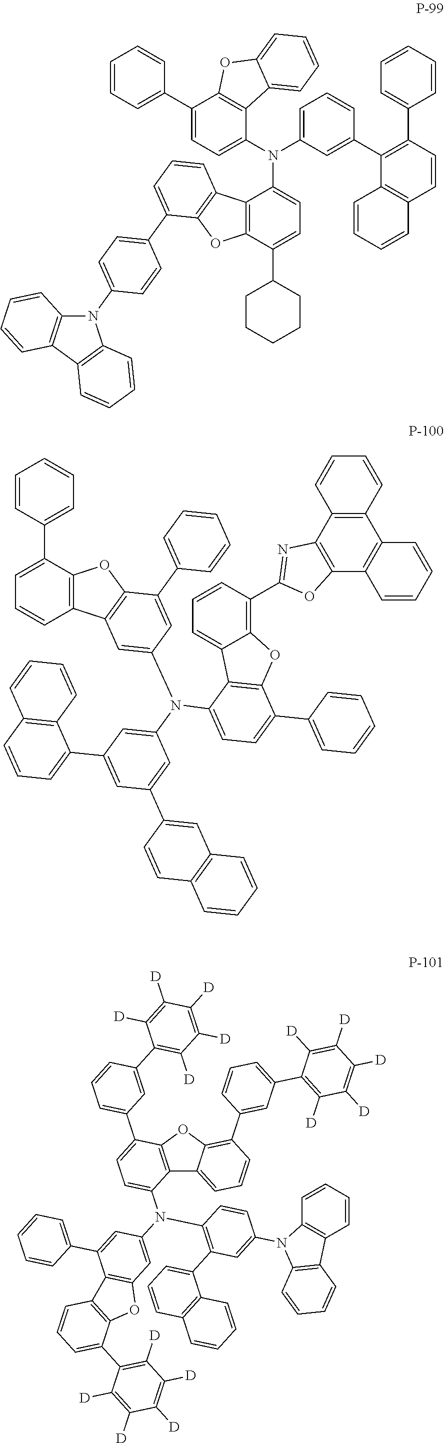

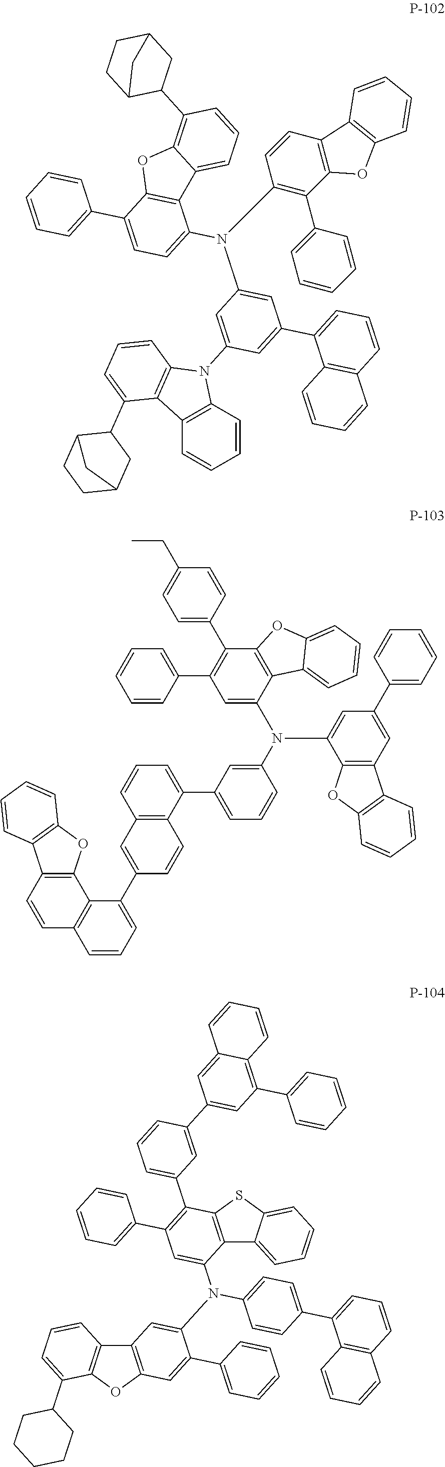 [SMILES: c1ccc(-c2c(N(c3cc(-c4cccc5ccccc45)cc(-n4c5ccccc5c5c(C6CC7CCC6C7)cccc54)c3)c3ccc(-c4ccccc4)c4oc5c(C6CC7CCC6C7)cccc5c34)ccc3c2oc2ccccc23)cc1 ; CCc1ccc(-c2c(-c3ccccc3)cc(N(c3cccc(-c4cccc5cc(-c6cccc7ccc8c9ccccc9oc8c67)ccc45)c3)c3cc(-c4ccccc4)cc4c3oc3ccccc34)c3c2oc2ccccc23)cc1 ; c1ccc(-c2cc3oc4c(C5CCCCC5)cccc4c3cc2N(c2ccc(-c3cccc4ccccc34)cc2)c2cc(-c3ccccc3)c(-c3cccc(-c4cc(-c5ccccc5)c5ccccc5c4)c3)c3sc4ccccc4c23)cc1]

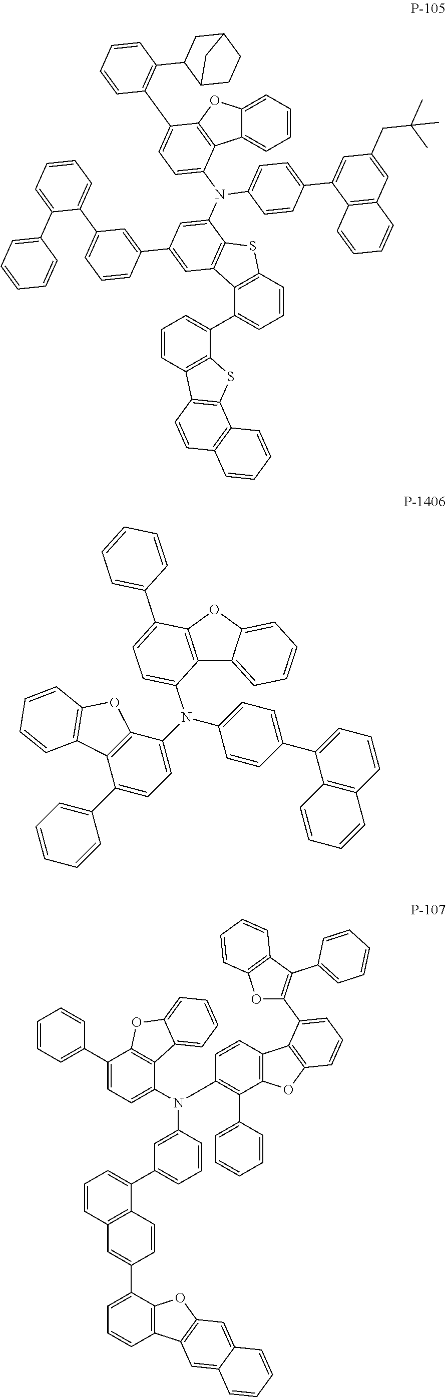 [SMILES: CC(C)(C)Cc1cc(-c2ccc(N(c3cc(-c4cccc(-c5ccccc5-c5ccccc5)c4)cc4c3sc3cccc(-c5cccc6c5sc5c7ccccc7ccc65)c34)c3ccc(-c4ccccc4C4CC5CCC4C5)c4oc5ccccc5c34)cc2)c2ccccc2c1 ; c1ccc(-c2ccc(N(c3ccc(-c4cccc5ccccc45)cc3)c3ccc(-c4ccccc4)c4c3oc3ccccc34)c3c2oc2ccccc23)cc1 ; c1ccc(-c2c(-c3cccc4oc5c(-c6ccccc6)c(N(c6cccc(-c7cccc8cc(-c9cccc%10c9oc9cc%11ccccc%11cc9%10)ccc78)c6)c6ccc(-c7ccccc7)c7oc8ccccc8c67)ccc5c34)oc3ccccc23)cc1]

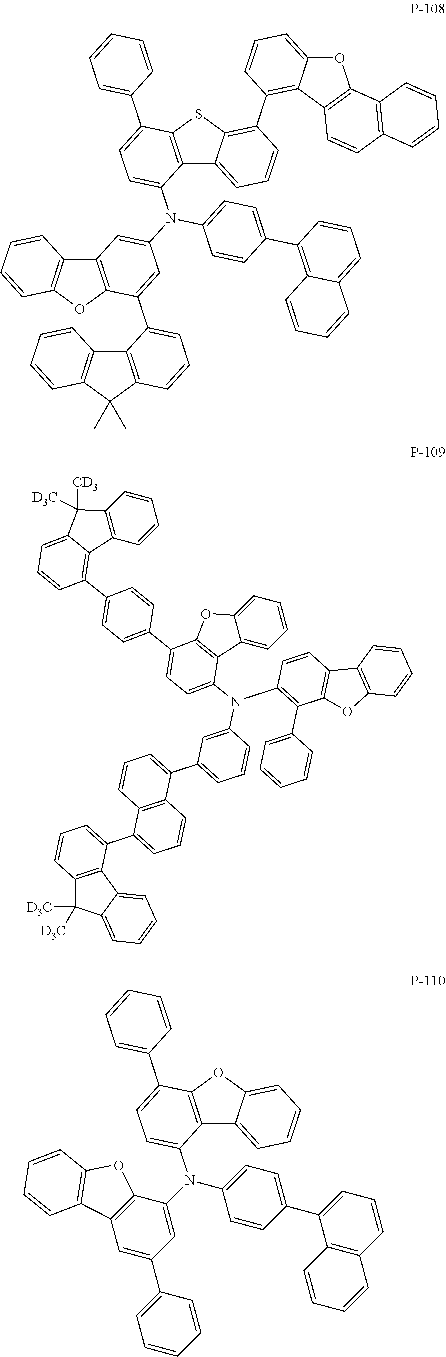 [SMILES: CC1(C)c2ccccc2-c2c(-c3cc(N(c4ccc(-c5cccc6ccccc56)cc4)c4ccc(-c5ccccc5)c5sc6c(-c7cccc8oc9c%10ccccc%10ccc9c78)cccc6c45)cc4c3oc3ccccc34)cccc21 ; [2H]C([2H])([2H])C1(C([2H])([2H])[2H])c2ccccc2-c2c(-c3ccc(-c4ccc(N(c5cccc(-c6cccc7c(-c8cccc9c8-c8ccccc8C9(C([2H])([2H])[2H])C([2H])([2H])[2H])cccc67)c5)c5ccc6c(oc7ccccc76)c5-c5ccccc5)c5c4oc4ccccc45)cc3)cccc21 ; c1ccc(-c2cc(N(c3ccc(-c4cccc5ccccc45)cc3)c3ccc(-c4ccccc4)c4oc5ccccc5c34)c3oc4ccccc4c3c2)cc1]

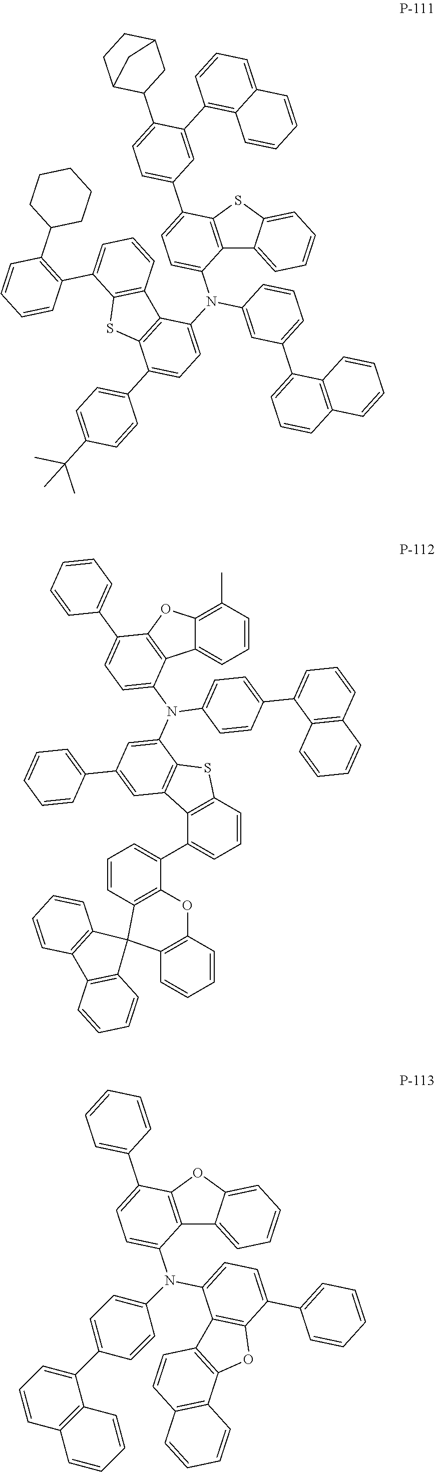 [SMILES: CC(C)(C)c1ccc(-c2ccc(N(c3cccc(-c4cccc5ccccc45)c3)c3ccc(-c4ccc(C5CC6CCC5C6)c(-c5cccc6ccccc56)c4)c4sc5ccccc5c34)c3c2sc2c(-c4ccccc4C4CCCCC4)cccc23)cc1 ; Cc1cccc2c1oc1c(-c3ccccc3)ccc(N(c3ccc(-c4cccc5ccccc45)cc3)c3cc(-c4ccccc4)cc4c3sc3cccc(-c5cccc6c5Oc5ccccc5C65c6ccccc6-c6ccccc65)c34)c12 ; c1ccc(-c2ccc(N(c3ccc(-c4cccc5ccccc45)cc3)c3ccc(-c4ccccc4)c4oc5c6ccccc6ccc5c34)c3c2oc2ccccc23)cc1]

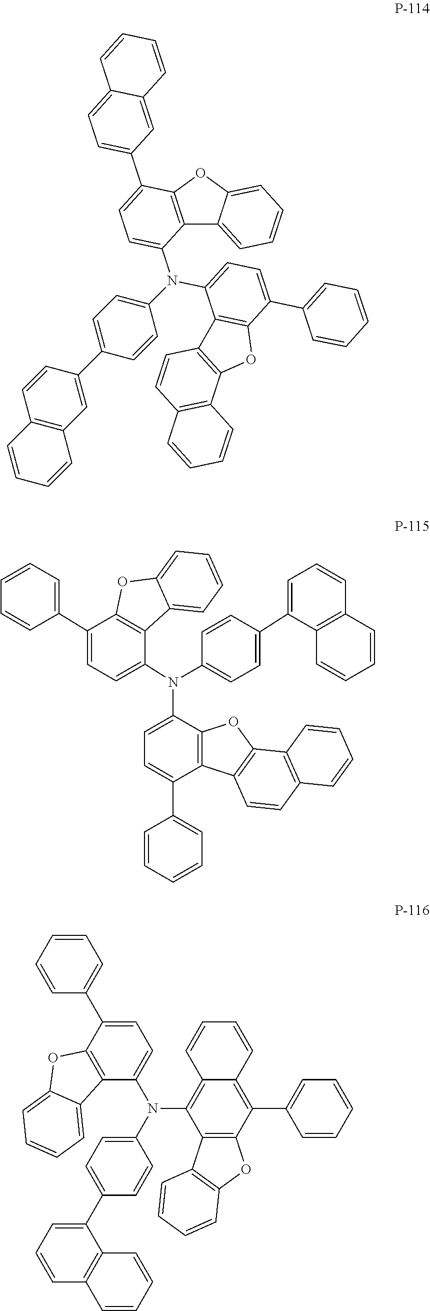 [SMILES: c1ccc(-c2ccc(N(c3ccc(-c4ccc5ccccc5c4)cc3)c3ccc(-c4ccc5ccccc5c4)c4oc5ccccc5c34)c3c2oc2c4ccccc4ccc23)cc1 ; c1ccc(-c2ccc(N(c3ccc(-c4cccc5ccccc45)cc3)c3ccc(-c4ccccc4)c4c3oc3c5ccccc5ccc34)c3c2oc2ccccc23)cc1 ; c1ccc(-c2ccc(N(c3ccc(-c4cccc5ccccc45)cc3)c3c4ccccc4c(-c4ccccc4)c4oc5ccccc5c34)c3c2oc2ccccc23)cc1]

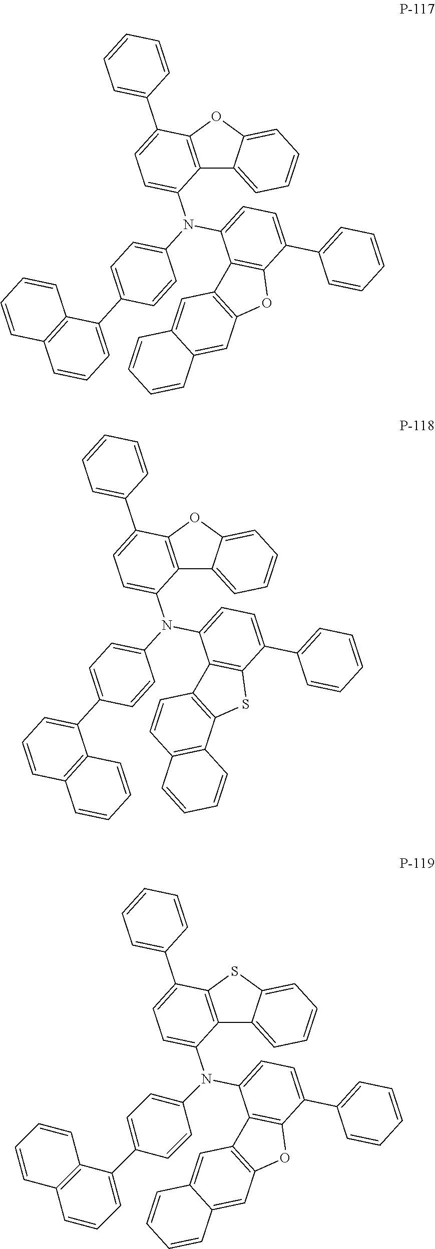 [SMILES: c1ccc(-c2ccc(N(c3ccc(-c4cccc5ccccc45)cc3)c3ccc(-c4ccccc4)c4oc5cc6ccccc6cc5c34)c3c2oc2ccccc23)cc1 ; c1ccc(-c2ccc(N(c3ccc(-c4cccc5ccccc45)cc3)c3ccc(-c4ccccc4)c4sc5c6ccccc6ccc5c34)c3c2oc2ccccc23)cc1 ; c1ccc(-c2ccc(N(c3ccc(-c4cccc5ccccc45)cc3)c3ccc(-c4ccccc4)c4sc5ccccc5c34)c3c2oc2cc4ccccc4cc23)cc1]

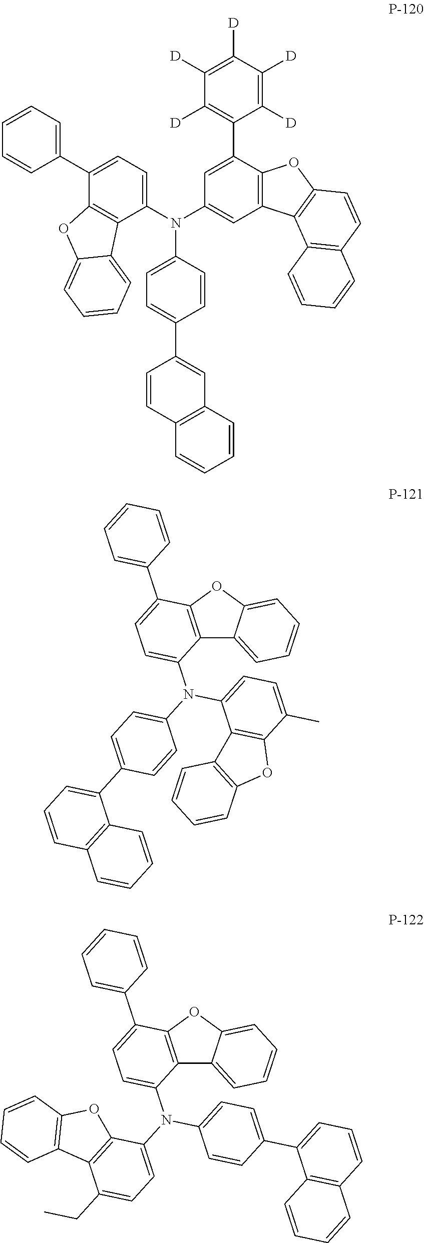 [SMILES: [2H]c1c([2H])c([2H])c(-c2cc(N(c3ccc(-c4ccc5ccccc5c4)cc3)c3ccc(-c4ccccc4)c4oc5ccccc5c34)cc3c2oc2ccc4ccccc4c23)c([2H])c1[2H] ; Cc1ccc(N(c2ccc(-c3cccc4ccccc34)cc2)c2ccc(-c3ccccc3)c3oc4ccccc4c23)c2c1oc1ccccc12 ; CCc1ccc(N(c2ccc(-c3cccc4ccccc34)cc2)c2ccc(-c3ccccc3)c3oc4ccccc4c23)c2oc3ccccc3c12]

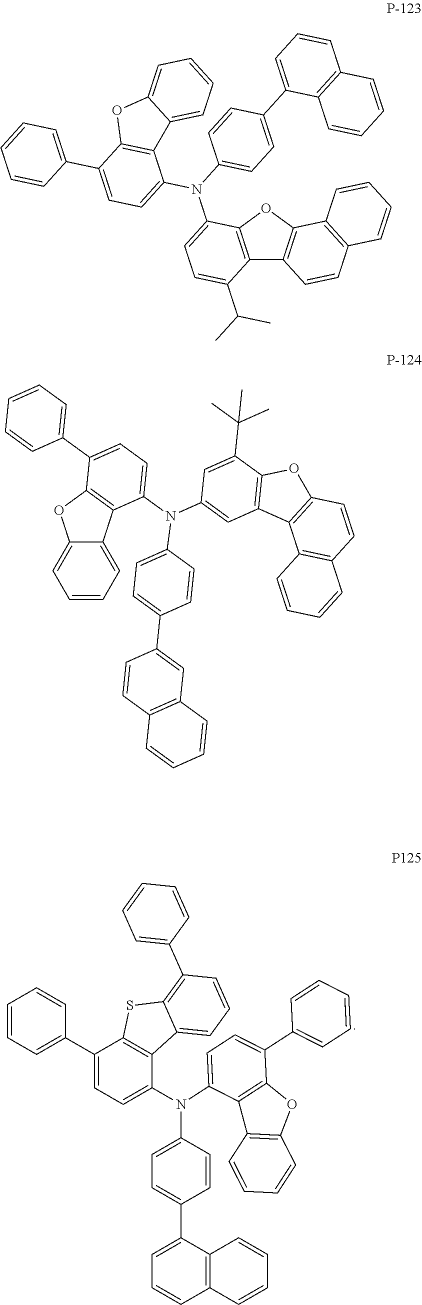 [SMILES: CC(C)c1ccc(N(c2ccc(-c3cccc4ccccc34)cc2)c2ccc(-c3ccccc3)c3oc4ccccc4c23)c2oc3c4ccccc4ccc3c12 ; CC(C)(C)c1cc(N(c2ccc(-c3ccc4ccccc4c3)cc2)c2ccc(-c3ccccc3)c3oc4ccccc4c23)cc2c1oc1ccc3ccccc3c12 ; c1ccc(-c2ccc(N(c3ccc(-c4cccc5ccccc45)cc3)c3ccc(-c4ccccc4)c4sc5c(-c6ccccc6)cccc5c34)c3c2oc2ccccc23)cc1]

In another aspect, the present invention provides a method for reusing a compound of Formula 1 comprising: recovering a crude organic light emitting material comprising the compound of Formula 1 from a deposition apparatus used in the process for depositing the organic emitting material to prepare an organic light emitting device; removing impurities from the crude organic light emitting material; recovering the organic light emitting material after the impurities are removed; and purifying the recovered organic light emitting material to have a purity of 99.9% or higher.

The step of removing impurities from the crude organic light emitting material recovered from the deposition apparatus may preferably comprise performing a pre-purification process to obtain a purity of 98% or more by recrystallization in a recrystallization solvent.

The recrystallization solvent may be preferably a polar solvent having a polarity index (PI) of 5.5 to 7.2.

The recrystallization solvent may preferably be used by mixing a polar solvent having a polarity value of 5.5 to 7.2 and a non-polar solvent having a polarity value of 2.0 to 4.7.

When a mixture of a polar solvent and a non-polar solvent is used, the recrystallization solvent may be used in an amount of 15% (v/v) or less of the non-polar solvent compared to the polar solvent.

The recrystallization solvent is preferably a single solvent of N-Methylpyrrolidone (NMP); or a polar solvent mixed any one selected from the group consisting of 1,3-Dimethyl-2-imidazolidinone, 2-pyrrolidone, N, N-Dimethyl formamide, Dimethyl acetamide, and Dimethyl sulfoxide to the N-Methylpyrrolidone; or alone; or mixed non-polar solvents; selected from the group consisting of Toluene, Dichloromethane (DCM), Dichloroethane (DCE), Tetrahydrofuran (THF), Chloroform, Ethyl acetate and Butanone; or a mixture of a polar solvent and a non-polar solvent.

The pre-purification process may comprise a step of precipitating crystals of by cooling to 0° C. to 5° C. after dissolving the crude organic light emitting material recovered from the deposition apparatus in a polar solvent at 90° C. to 120° C.

The pre-purification process may comprise a step of precipitating crystals by cooling to 35° C. to 40° C., adding a non-polar solvent, and then cooling to 0° C. to 5° C. after dissolving the crude organic light emitting material recovered from the deposition apparatus in a polar solvent at 90° C. to 120° C.

The pre-purification process may comprise a step of precipitating crystals while concentrating the solvent and removing the non-polar solvent, after dissolving the crude organic light emitting material recovered from the deposition apparatus in a non-polar solvent.

The pre-purification process may comprise a step of recrystallizing again with a non-polar solvent after recrystallizing first with a polar solvent.

The step of purifying the recovered impurities to a purity of 99.9% or higher may comprise performing an adsorption separation process to adsorb and remove impurities by adsorbing on the adsorbent.

The adsorbent may be activated carbon, silica gel, alumina, or a material for known adsorption purposes.

The step of purifying the recovered impurities to a purity of 99.9% or higher may comprise performing sublimation purification.

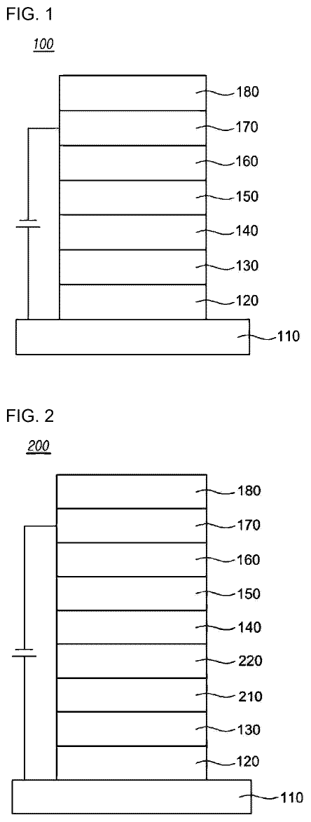

Referring to FIG. 1, the organic electronic element (100) according to the present invention comprises a first electrode (110), a second electrode (170), an organic material layer comprising single compound or 2 or more compounds represented by Formula 1 between the first electrode (110) and the second electrode (170). Wherein, the first electrode (110) may be an anode or a positive electrode, and the second electrode (170) may be a cathode or a negative electrode. In the case of an inverted organic electronic element, the first electrode may be a cathode, and the second electrode may be an anode.

The organic material layer may sequentially comprise a hole injection layer (120), a hole transport layer (130), an emitting layer (140), an electron transport layer (150), and an electron injection layer (160) on the first electrode (110). Here, the remaining layers except the emitting layer (140) may not be formed. The organic material layer may further comprise a hole blocking layer, an electron blocking layer, an emitting-auxiliary layer (220), a buffer layer (210), etc., and the electron transport layer (150), etc. may serve as a hole blocking layer (see FIG. 2).

Also, the organic electronic element according to an embodiment of the present invention may further include a protective layer or a light efficiency enhancing layer (180). Wherein the light efficiency enhancing layer is formed on one of both surfaces of the first electrode that is not in contact with the organic material layer or on one of both surfaces of the second electrode that is not in contact with the organic material layer. The compound according to an embodiment of the present invention applied to the organic material layer may be used as a material for a hole injection layer (120), a hole transport layer (130), an emitting-auxiliary layer (220), an electron transport auxiliary layer, an electron transport layer (150), an electron injection layer (160), a host or dopant of an emitting layer (140), or the light efficiency enhancing layer. Preferably, for example, a compound according to Formula 1 of the present invention can be used as a material of an emitting-auxiliary layer.

The organic material layer may comprise 2 or more stacks comprising a hole transport layer, an emitting layer and an electron transport layer sequentially formed on the anode, and may further comprise a charge generation layer formed between the 2 or more stacks (see FIG. 3).

Otherwise, even if the same core is used, the band gap, the electrical characteristics, the interface characteristics, etc. may vary depending on which substituent is bonded at which position, therefore the choice of core and the combination of sub-substituents associated therewith is also very important, and in particular, when the optimal combination of energy levels and T1 values, and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (120), the hole transport layer (130), the emitting layer (140), the electron transport layer (150), and the electron injection layer (160) is formed thereon, and then a material that can be used as a cathode is deposited thereon.

Also, the present invention provides the organic electronic element wherein the organic material layer is formed by one of a spin coating process, a nozzle printing process, an inkjet printing process, a slot coating process, a dip coating process or a roll-to-roll process, and the organic material layer comprises the compound as an electron transport material.

As another specific example, a compound of the same or different types represented by Formula 1 is mixed and used in the organic material layer.

Additionally, the present invention provides an emitting-auxiliary layer composition comprising a compound represented by Formula 1, and provides an organic electronic element comprising the emitting-auxiliary layer.

Also, the present invention also provides an electronic device comprising a display device comprising the organic electronic element; and a control unit for driving the display device.

According to another aspect, the present invention provides a display device wherein the organic electronic element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis examples of the compound represented by Formula 1 of the present invention, and preparation examples of the organic electronic element will be described in detail by way of example, but are not limited to the following examples.

### Synthesis Example 1

The compound (final products) represented by Formula 1 according to the present invention can be synthesized by a reaction as in Reaction Scheme 1, but is not limited thereto. (Hal¹=Cl, Br or I)

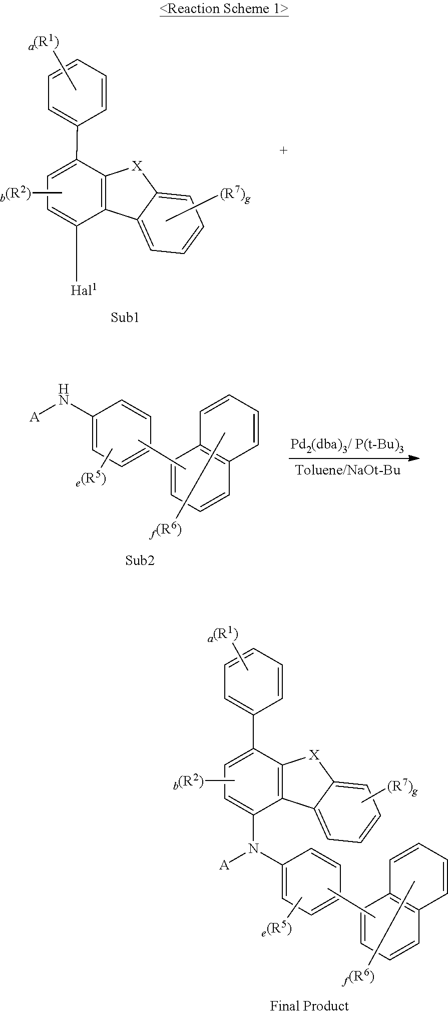

### I. Synthesis of Final Product

### 1. Synthesis of P-1

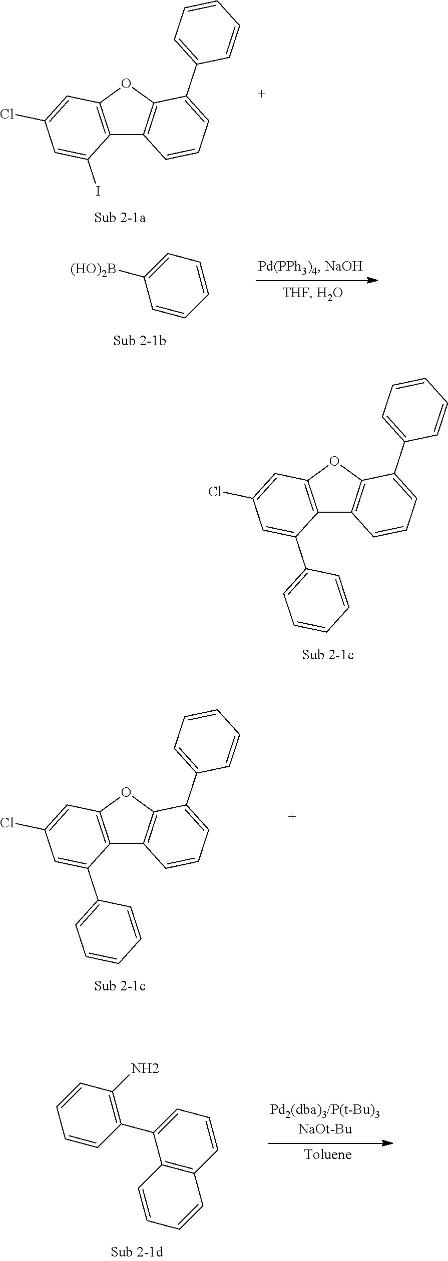 [SMILES: Clc1cc(I)c2c(c1)oc1c(-c3ccccc3)cccc12 ; OB(O)c1ccccc1 ; Clc1cc(-c2ccccc2)c2c(c1)oc1c(-c3ccccc3)cccc12 ; Clc1cc(-c2ccccc2)c2c(c1)oc1c(-c3ccccc3)cccc12 ; Nc1ccccc1-c1cccc2ccccc12]

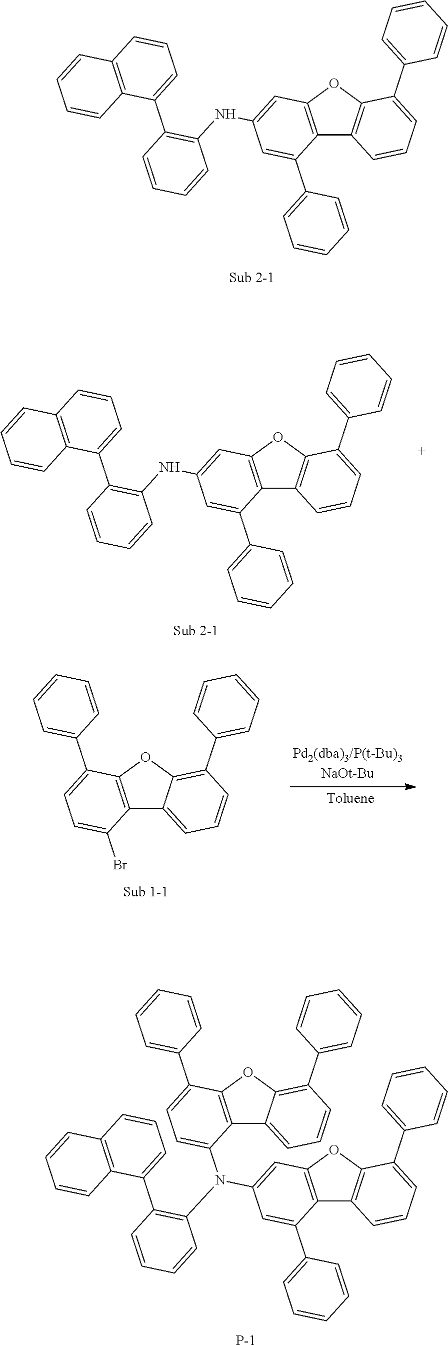

### 1) Synthesis of Sub 2-1c

Sub 2-1a (50.0 g, 123.6 mmol) was dissolved in THF (Tetrahydrofuran) (mL) in a round-bottom flask, Sub 2-1b (15.1 g, 123.6 mmol), K₂CO₃ (51.2 g, 138.2 mmol), Pd(PPh₃)₄ (8.57 g, 7.41 mmol) and water (309 mL) were added, and stirred at 80° C. After the reaction was completed, the mixture was extracted with CH₂Cl₂ and water, the organic layer was dried over MgSO₄ and concentrated, and the resulting compound was recrystallized using a silicagel column to obtain 34.4 g of the product (yield: 78%).

### 2) Synthesis of Sub 2-1

Sub 2-1c (30.0 g, 84.5 mmol) was dissolved in Toluene (423 mL) in a round-bottom flask, Sub 2-1d (18.5 g, 84.5 mmol), Pd₂(dba)₃ (2.32 g, 2.54 mmol), P(t-Bu)₃ (1.03 g, 5.07 mmol), NaOt-Bu (16.3 g, 169.1 mmol) were added, and stirred at 80° C. After the reaction was completed, the mixture was extracted with CH₂Cl₂ and water, the organic layer was dried over MgSO₄ and concentrated, and the resulting compound was recrystallized using a silicagel column to obtain 31.4 g of the product (yield: 69.0%).

### 3) Synthesis of P-1

Sub 2-1 (10.0 g, 18.6 mmol) was dissolved in Toluene (93 mL) in a round-bottom flask, Sub 1-1 (7.4 g, 18.6 mmol), Pd₂(dba)₃ (0.51 g, 0.56 mmol), P(t-Bu)₃ (0.23 g, 1.12 mmol), NaOt-Bu (3.6 g, 67.2 mmol) were added, and 11.2 g of the product (yield: 70.2%) was obtained using the synthetic method of Sub 2-1.

### 2. Synthesis of P-9

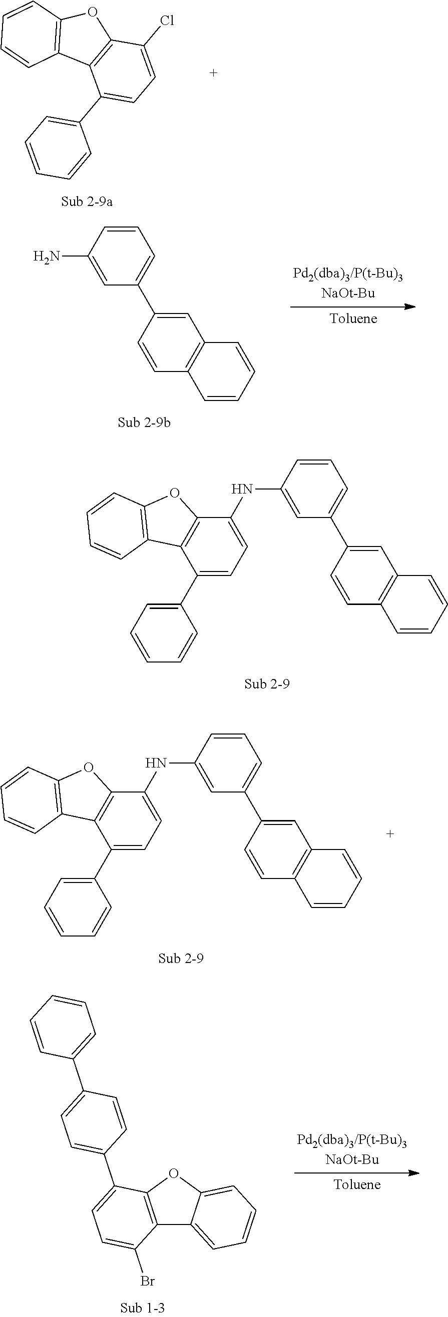 [SMILES: Clc1ccc(-c2ccccc2)c2c1oc1ccccc12 ; Nc1cccc(-c2ccc3ccccc3c2)c1 ; c1ccc(-c2ccc(Nc3cccc(-c4ccc5ccccc5c4)c3)c3oc4ccccc4c23)cc1 ; c1ccc(-c2ccc(Nc3cccc(-c4ccc5ccccc5c4)c3)c3oc4ccccc4c23)cc1 ; Brc1ccc(-c2ccc(-c3ccccc3)cc2)c2oc3ccccc3c12]

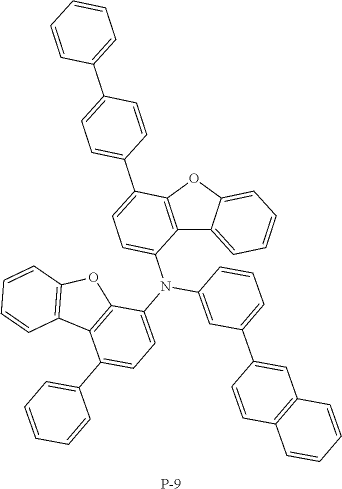

### 1) Synthesis of Sub 2-9

Sub 2-9a (50.0 g, 179.4 mmol) was dissolved in Toluene (897 mL) in a round-bottom flask, Sub 2-9b (39.3 g, 179.4 mmol), Pd₂(dba)₃ (4.93 g, 5.38 mmol), P(t-Bu)₃ (2.18 g, 10.76 mmol), NaOt-Bu (34.5 g, 358.8 mmol) were added, and 56.7 g of the product (yield: 68.5%) was obtained using the synthetic method of Sub 2-1.

### 2) Synthesis of P-9

Sub 2-9 (10 g, 21.7 mmol) was dissolved in Toluene (108 mL) in a round-bottom flask, Sub 1-3 (8.7 g, 21.7 mmol), Pd₂(dba)₃ (0.60 g, 0.65 mmol), P(t-Bu)₃ (0.26 g, 1.30 mmol), NaOt-Bu (4.2 g, 43.3 mmol) were added, and 12.0 g of the product (yield: 71.2%) was obtained using the synthetic method of Sub 2-1.

### 3. Synthesis of P-25

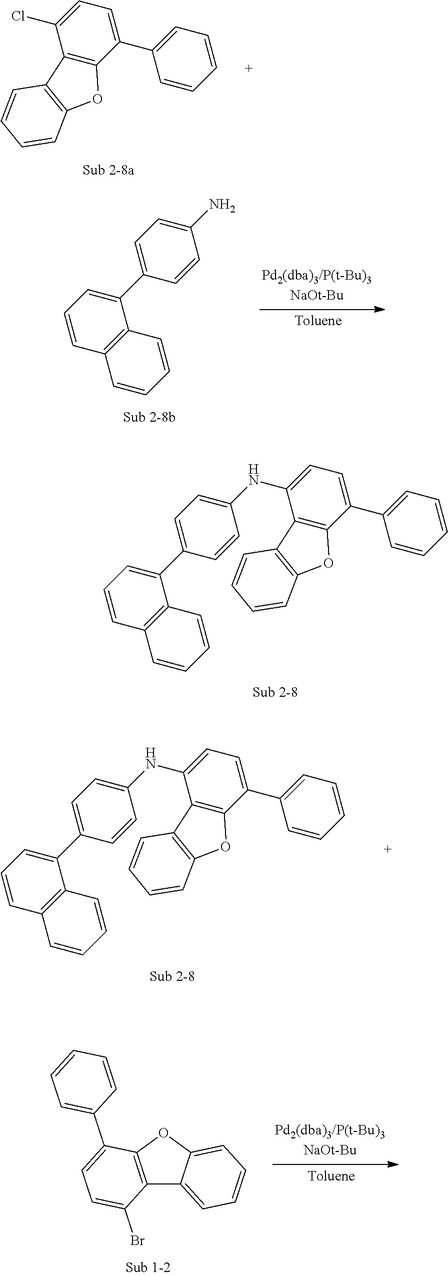 [SMILES: Clc1ccc(-c2ccccc2)c2oc3ccccc3c12 ; Nc1ccc(-c2cccc3ccccc23)cc1 ; c1ccc(-c2ccc(Nc3ccc(-c4cccc5ccccc45)cc3)c3c2oc2ccccc23)cc1 ; c1ccc(-c2ccc(Nc3ccc(-c4cccc5ccccc45)cc3)c3c2oc2ccccc23)cc1 ; Brc1ccc(-c2ccccc2)c2oc3ccccc3c12]

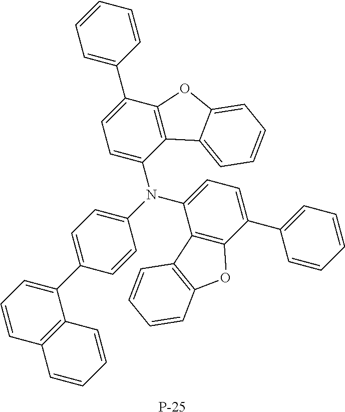

### 1) Synthesis of Sub 2-8

Sub 2-18a (50.0 g, 179.4 mmol) was dissolved in Toluene (897 mL) in a round-bottom flask, Sub 2-18b (39.3 g, 179.4 mmol), Pd₂(dba)₃ (4.93 g, 5.38 mmol), P(t-Bu)₃ (2.18 g, 10.76 mmol), NaOt-Bu (34.5 g, 358.8 mmol) were added, and 56.3 g of the product (yield: 68%) was obtained using the synthetic method of Sub 2-1.

### 2) Synthesis of P-25

Sub 2-18 (10 g, 21.7 mmol) was dissolved in Toluene (108 mL) in a round-bottom flask, Sub 1-2 (7.0 g, 21.7 mmol), Pd₂(dba)₃ (0.6 g, 0.65 mmol), P(t-Bu)₃ (0.26 g, 1.30 mmol), NaOt-Bu (4.2 g, 43.3 mmol) were added, and 10.6 g of the product (yield: 69.2%) was obtained using the synthetic method of Sub 2-1.

### 4. Synthesis of P-32

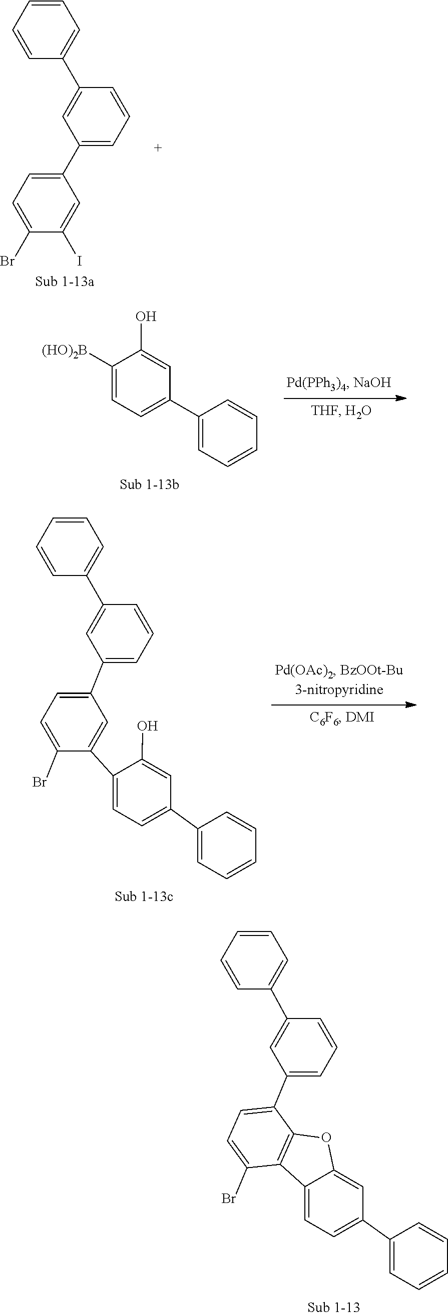 [SMILES: Brc1ccc(-c2cccc(-c3ccccc3)c2)cc1I ; OB(O)c1ccc(-c2ccccc2)cc1O ; Oc1cc(-c2ccccc2)ccc1-c1cc(-c2cccc(-c3ccccc3)c2)ccc1Br ; Brc1ccc(-c2cccc(-c3ccccc3)c2)c2oc3cc(-c4ccccc4)ccc3c12]

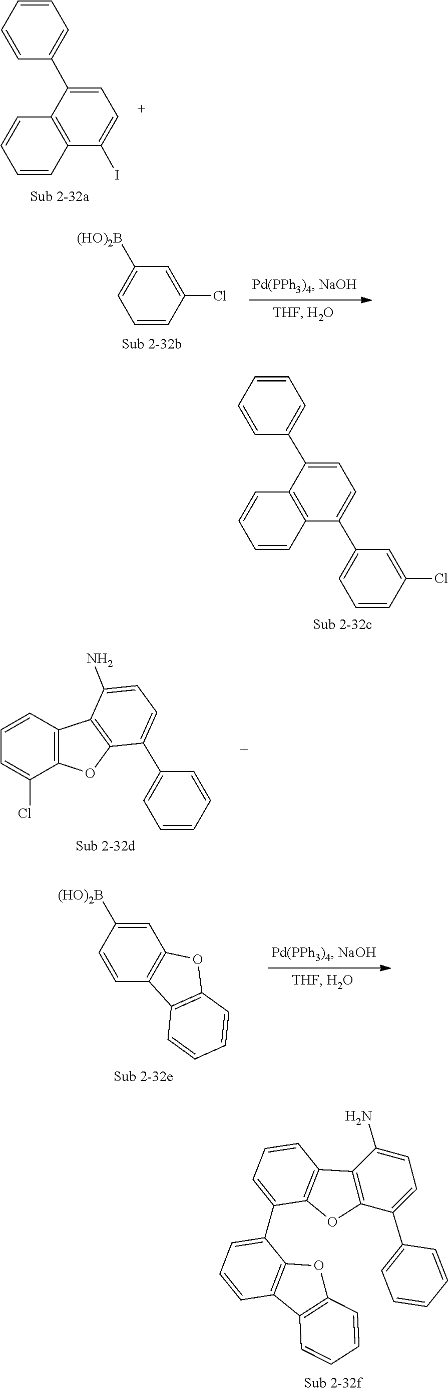 [SMILES: Ic1ccc(-c2ccccc2)c2ccccc12 ; OB(O)c1cccc(Cl)c1 ; Clc1cccc(-c2ccc(-c3ccccc3)c3ccccc23)c1 ; Nc1ccc(-c2ccccc2)c2oc3c(Cl)cccc3c12 ; OB(O)c1ccc2c(c1)oc1ccccc12 ; Nc1ccc(-c2ccccc2)c2oc3c(-c4cccc5c4oc4ccccc45)cccc3c12]

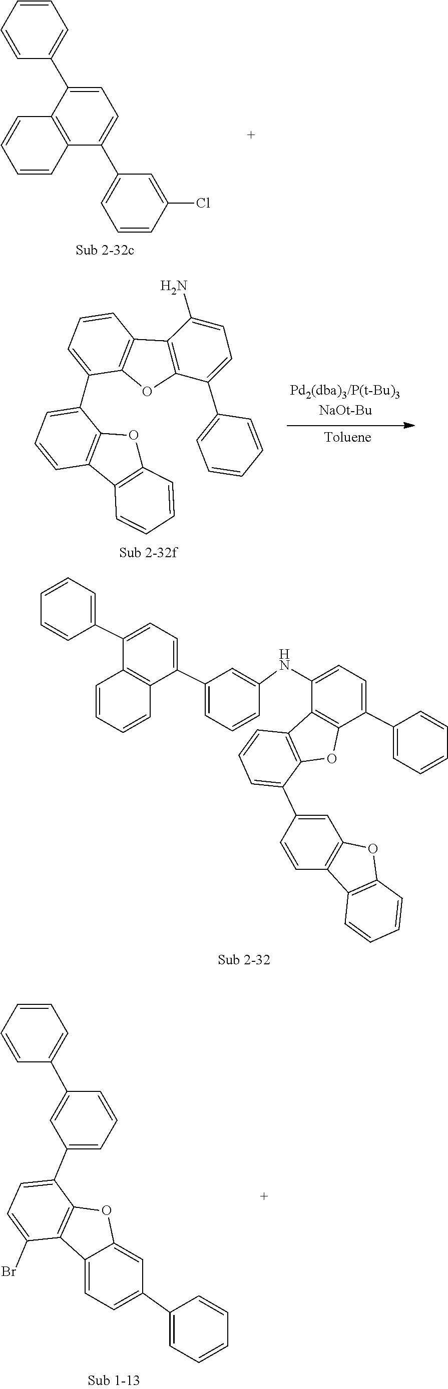 [SMILES: Clc1cccc(-c2ccc(-c3ccccc3)c3ccccc23)c1 ; Nc1ccc(-c2ccccc2)c2oc3c(-c4cccc5c4oc4ccccc45)cccc3c12 ; c1ccc(-c2ccc(-c3cccc(Nc4ccc(-c5ccccc5)c5oc6c(-c7ccc8c(c7)oc7ccccc78)cccc6c45)c3)c3ccccc23)cc1 ; Brc1ccc(-c2cccc(-c3ccccc3)c2)c2oc3cc(-c4ccccc4)ccc3c12]

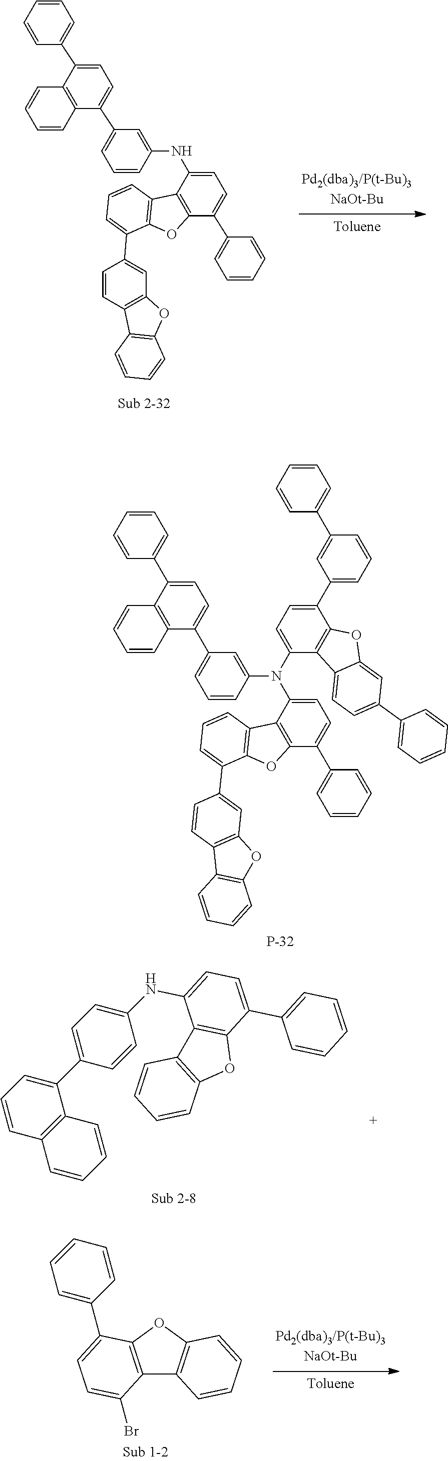

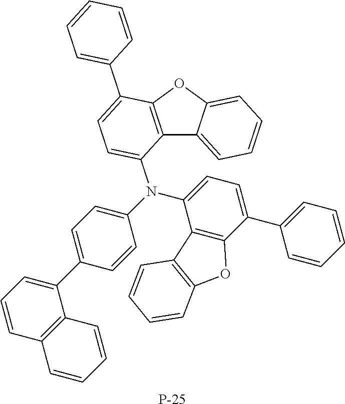

### 1) Synthesis of Sub 1-13c

Sub 1-13a (50 g, 114.9 mmol) was dissolved in THF (575 mL) in a round-bottom flask, Sub 1-13b (24.6 g, 114.9 mmol), NaOH (13.8 g, 344.7 mmol), Pd(PPh₃)₄ (7.97 g, 6.89 mmol), water (287 mL) were added, and 43.9 g of the product (yield: 80.0%) was obtained using the synthetic method of Sub 2-1c.

### 2) Synthesis of 1-13

Pd(OAc)₂ (1.03 g, 4.60 mmol), 3-nitropyridine (0.570 g, 4.60 mmol), BzOOt-Bu (tert-butyl peroxybenzoate) (35.7 g, 183.9 mmol), C₆F₆ (hexafluorobenzene) (137 mL), DMI (N,N′-dimethylimidazolidinone) (92 mL) were added to Sub1-13c (43.9 g, 91.9 mmol) and refluxed at 90° C. for 3 hours. When the reaction was complete, the reaction product was cooled to room temperature and extracted with ethyl acetate and water. The organic layer was dried over MgSO₄ and concentrated, and the resulting compound was recrystallized using a silicagel column to obtain 27.1 g of the product (yield: 62.0%).

### 3) Synthesis of Sub 2-32c

Sub 2-27a (50.0 g, 151.4 mmol) was dissolved in THF (Tetrahydrofuran) (757 mL) in a round-bottom flask, Sub 2-27b (23.7 g, 151.4 mmol), K₂CO₃ (62.8 g, 454.3 mmol), Pd(PPh₃)₄ (10.5 g, 9.09 mmol), water (379 mL) were added, and 37.2 g of the product (yield: 78.0%) was obtained using the synthetic method of Sub 2-1c.

### 4) Synthesis of Sub 2-32f

Sub 2-27d (50.0 g, 170.2 mmol) was dissolved in THF (Tetrahydrofuran) (851 mL) in a round-bottom flask, Sub 2-27e (36.1 g, 170.2 mmol), K₂CO₃ (70.6 g, 510.6 mmol), Pd(PPh₃)₄ (11.80 g, 10.21 mmol), water (426 mL) were added, and 54.3 g of the product (yield: 75.0%) was obtained using the synthetic method of Sub 2-1c.

### 5) Synthesis of Sub 2-32

Sub 2-27c (30.0 g, 63.1 mmol) was dissolved in toluene (316 mL) in a round-bottom flask, Sub 2-27f (26.9 g, 63.1 mmol), Pd₂(dba)₃ (1.73 g, 1.89 mmol), P(t-Bu)₃ (0.77 g, 3.79 mmol), NaOt-Bu (12.1 g, 126.2 mmol) were added, and 30.6 g of the product (yield: 68.8%) was obtained using the synthetic method of Sub 2-1.

### 6) Synthesis of P-32

Sub 1-13 (10 g, 21.0 mmol) was dissolved in toluene (105 mL) in a round-bottom flask, Sub 2-27 (14.8 g, 21.0 mmol), Pd₂(dba)₃ (0.58 g, 0.63 mmol), P(t-Bu)₃ (0.23 g, 1.26 mmol), NaOt-Bu (4.0 g, 42.1 mmol) were added, and 15.9 g of the product (yield: 68.8%) was obtained using the synthetic method of Sub 2-1.

### 5. Synthesis of P-42

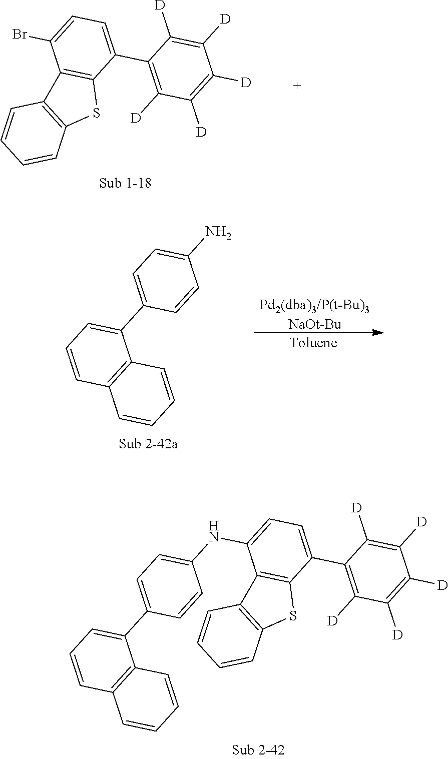

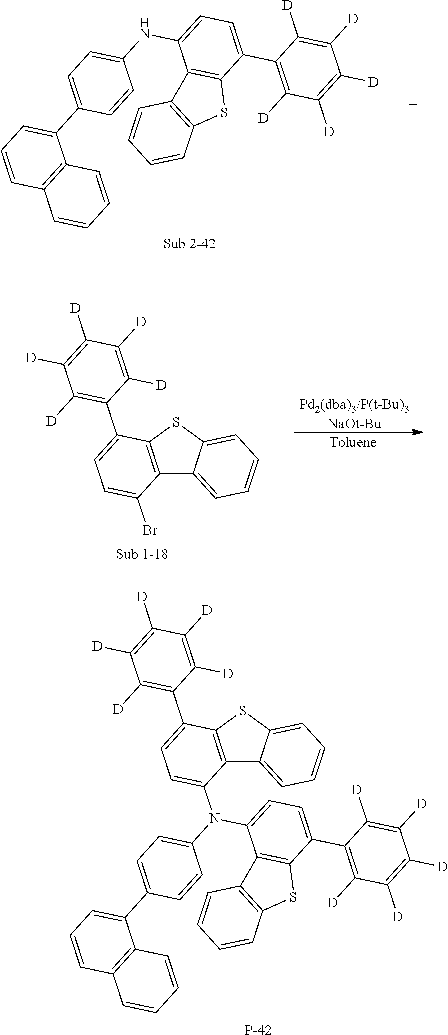

### 1) Synthesis of Sub 2-37

Sub 1-18 (50 g, 166.8 mmol) was dissolved in toluene (834 mL) in a round-bottom flask, Sub 2-18b (36.6 g, 166.8 mmol), Pd₂(dba)₃ (4.58 g, 5.0 mmol), P(t-Bu)₃ (2.02 g, 10.01 mmol), NaOt-Bu (32.1 g, 333.5 mmol) were added, and 57.3 g of the product (yield: 71.2%) was obtained using the synthetic method of Sub 2-1.

### 2) Synthesis of P-42

Sub 2-37 (10 g, 20.7 mmol) was dissolved in toluene (104 mL) in a round-bottom flask, Sub 1-18 (6.2 g, 20.7 mmol), Pd₂(dba)₃ (0.57 g, 0.62 mmol), P(t-Bu)₃ (0.25 g, 1.24 mmol), NaOt-Bu (4.0 g, 41.4 mmol) were added, and 10.7 g of the product (yield: 69.0%) was obtained using the synthetic method of Sub 2-1.

### 6. Synthesis of P-54

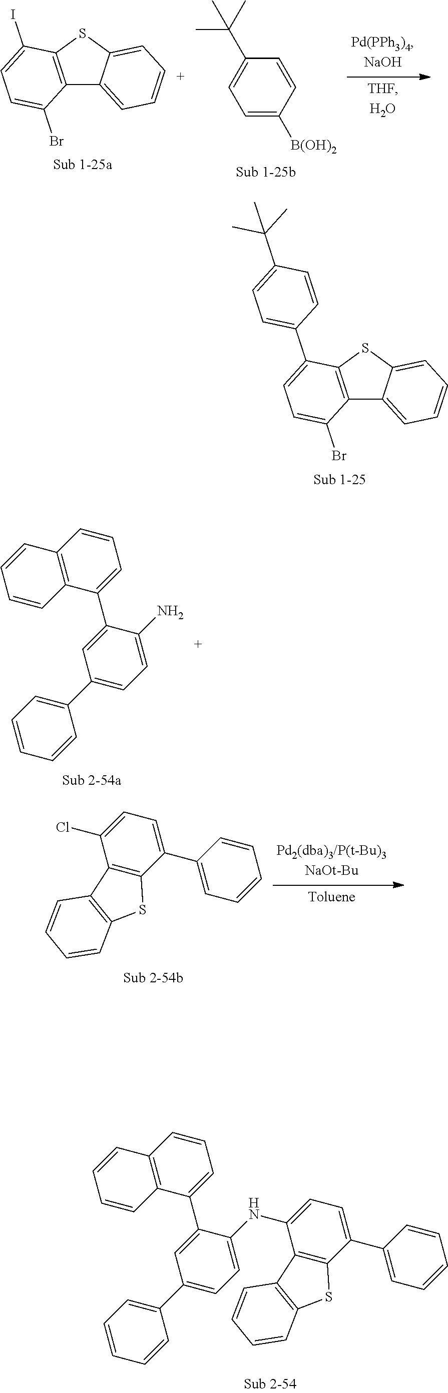 [SMILES: Brc1ccc(I)c2sc3ccccc3c12 ; CC(C)(C)c1ccc(B(O)O)cc1 ; CC(C)(C)c1ccc(-c2ccc(Br)c3c2sc2ccccc23)cc1 ; Nc1ccc(-c2ccccc2)cc1-c1cccc2ccccc12 ; Clc1ccc(-c2ccccc2)c2sc3ccccc3c12 ; c1ccc(-c2ccc(Nc3ccc(-c4ccccc4)c4sc5ccccc5c34)c(-c3cccc4ccccc34)c2)cc1]

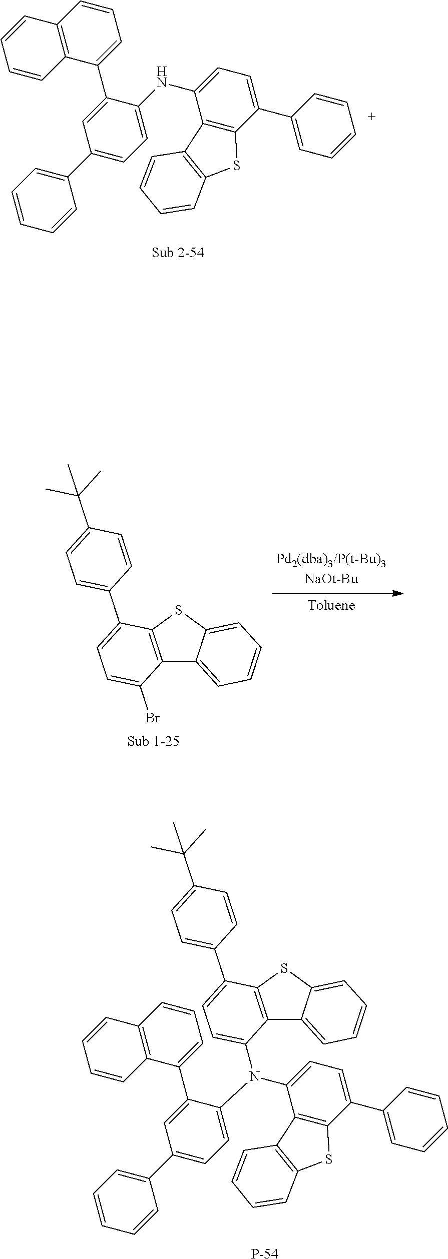

### 1) Synthesis of Sub 1-25

Sub 1-25a (50 g, 128.5 mmol) was dissolved in THF (Tetrahydrofuran) (643 mL) in a round-bottom flask, Sub 1-25b (22.9 g, 128.5 mmol), K₂CO₃ (53.3 g, 385.6 mmol), Pd(PPh₃)₄ (8.91 g, 7.71 mmol), water (321 mL) were added, and 40.4 g of the product (yield: 79.6%) was obtained using the synthetic method of Sub 2-1c.

### 2) Synthesis of Sub 2-54

Sub 2-49a (50 g, 169.3 mmol) was dissolved in toluene (846 mL) in a round-bottom flask, Sub 2-49b (49.9 g, 169.3 mmol), Pd₂(dba)₃ (4.65 g, 5.08 mmol), P(t-Bu)₃ (2.05 g, 10.16 mmol), NaOt-Bu (32.5 g, 338.5 mmol) were added, and 64.3 g of the product (yield: 68.6%) was obtained using the synthetic method of Sub 2-1.

### 3) Synthesis of P-54

Sub 2-49 (10 g, 18.1 mmol) was dissolved in toluene (90 mL) in a round-bottom flask, Sub 1-25 (7.1 g, 18.1 mmol), Pd₂(dba)₃ (0.50 g, 0.54 mmol), P(t-Bu)₃ (0.22 g, 1.08 mmol), NaOt-Bu (3.5 g, 36.1 mmol) were added, and 11.2 g of the product (yield: 71.5%) was obtained using the synthetic method of Sub 2-1.

### 7. Synthesis of P-66

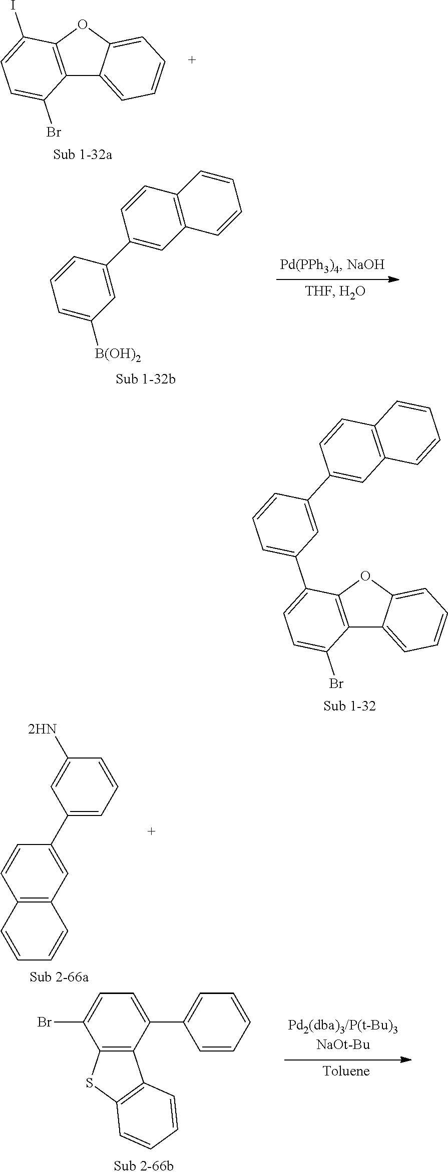

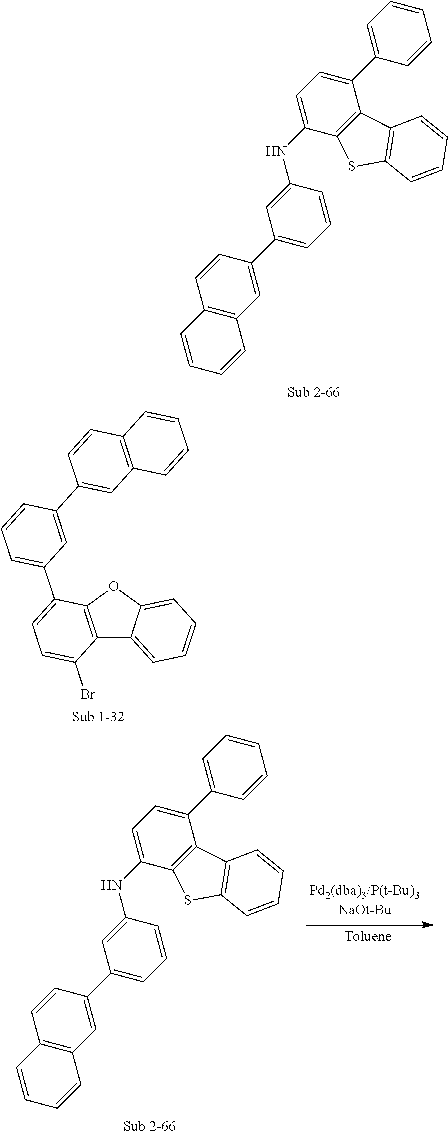 [SMILES: c1ccc(-c2ccc(Nc3cccc(-c4ccc5ccccc5c4)c3)c3sc4ccccc4c23)cc1 ; Brc1ccc(-c2cccc(-c3ccc4ccccc4c3)c2)c2oc3ccccc3c12 ; c1ccc(-c2ccc(Nc3cccc(-c4ccc5ccccc5c4)c3)c3sc4ccccc4c23)cc1]

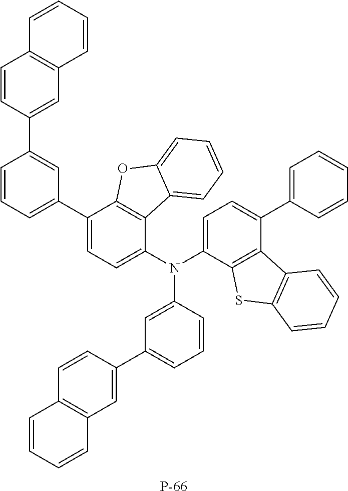

### 1) Synthesis of Sub 1-32

Sub 1-32a (50 g, 134.1 mmol) was dissolved in THF (Tetrahydrofuran) (670 mL) in a round-bottom flask, Sub 1-32b (33.3 g, 134.1 mmol), K₂CO₃ (55.6 g, 402.2 mmol), Pd(PPh₃)₄ (9.29 g, 8.04 mmol), water (335 mL) were added, and 47.9 g of the product (yield: 79.6%) was obtained using the synthetic method of Sub 2-1c.

### 2) Synthesis of Sub 2-66

Sub 2-59a (50 g, 228.0 mmol) was dissolved in toluene (1140 mL) in a round-bottom flask, Sub 2-59b (56.6 g, 228.0 mmol), Pd₂(dba)₃ (6.26 g, 6.84 mmol), P(t-Bu)₃ (2.77 g, 13.68 mmol), NaOt-Bu (43.8 g, 456.0 mmol) were added, and 76.3 g of the product (yield: 70.1%) was obtained using the synthetic method of Sub 2-1.

### 3) Synthesis of P-66

Sub 1-32 (10 g, 22.3 mmol) was dissolved in toluene (11 mL) in a round-bottom flask, Sub 2-59 (10.6 g, 22.3 mmol), Pd₂(dba)₃ (0.61 g, 0.67 mmol), P(t-Bu)₃ (0.27 g, 1.34 mmol), NaOt-Bu (4.3 g, 44.5 mmol) were added, and 7.6 g of the product (yield: 71.3%) was obtained using the synthetic method of Sub 2-1.

### 8. Synthesis of P-83

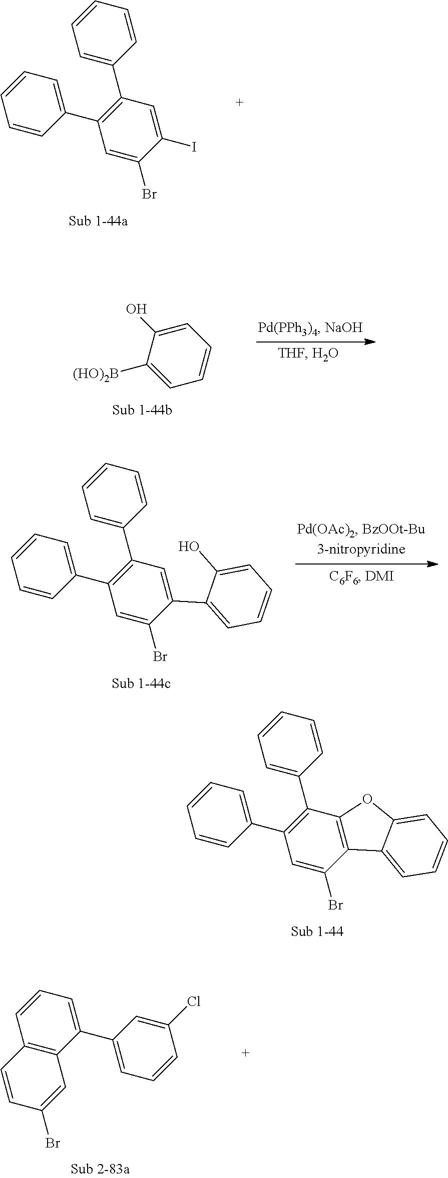 [SMILES: Brc1cc(-c2ccccc2)c(-c2ccccc2)cc1I ; OB(O)c1ccccc1O ; Oc1ccccc1-c1cc(-c2ccccc2)c(-c2ccccc2)cc1Br ; Brc1cc(-c2ccccc2)c(-c2ccccc2)c2oc3ccccc3c12 ; Clc1cccc(-c2cccc3ccc(Br)cc23)c1]

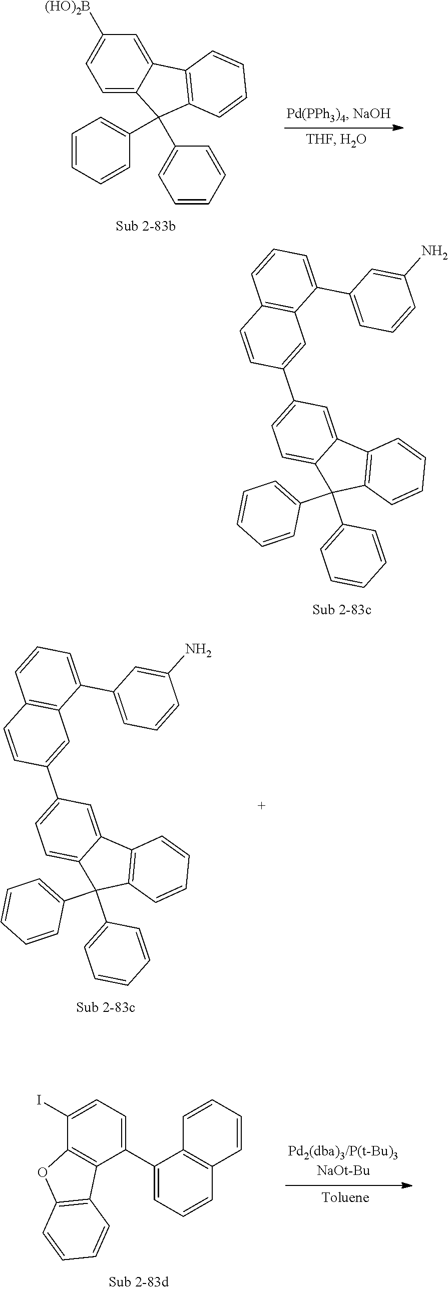 [SMILES: OB(O)c1ccc2c(c1)-c1ccccc1C2(c1ccccc1)c1ccccc1 ; Nc1cccc(-c2cccc3ccc(-c4ccc5c(c4)-c4ccccc4C5(c4ccccc4)c4ccccc4)cc23)c1 ; Nc1cccc(-c2cccc3ccc(-c4ccc5c(c4)-c4ccccc4C5(c4ccccc4)c4ccccc4)cc23)c1 ; Ic1ccc(-c2cccc3ccccc23)c2c1oc1ccccc12]

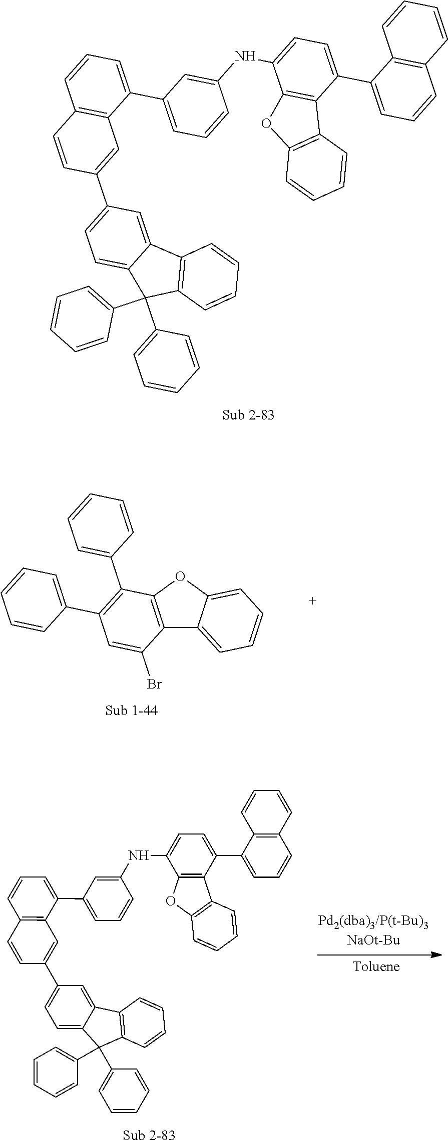 [SMILES: c1ccc(C2(c3ccccc3)c3ccccc3-c3cc(-c4ccc5cccc(-c6cccc(Nc7ccc(-c8cccc9ccccc89)c8c7oc7ccccc78)c6)c5c4)ccc32)cc1 ; Brc1cc(-c2ccccc2)c(-c2ccccc2)c2oc3ccccc3c12 ; c1ccc(C2(c3ccccc3)c3ccccc3-c3cc(-c4ccc5cccc(-c6cccc(Nc7ccc(-c8cccc9ccccc89)c8c7oc7ccccc78)c6)c5c4)ccc32)cc1]

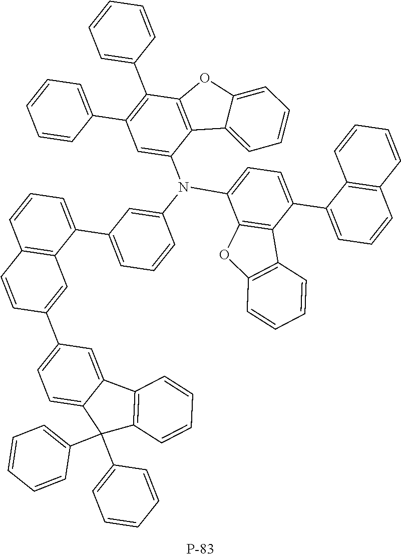

### 1) Synthesis of Sub 1-44c

Sub 1-44a (50 g, 114.9 mmol) was dissolved in THF (575 mL) in a round-bottom flask, Sub 1-44b (15.9 g, 114.9 mmol), NaOH (13.8 g, 344.7 mmol), Pd(PPh₃)₄ (7.97 g, 6.89 mmol), water (287 mL) were added, and 36.4 g of the product (yield: 79.0%) was obtained using the synthetic method of Sub 2-1c.

### 2) Synthesis of Sub 1-44

Pd(OAc)₂ (1.02 g, 4.54 mmol), 3-nitropyridine (0.563 g, 4.54 mmol), BzOOt-Bu (tert-butyl peroxybenzoate) (35.3 g, 181.6 mmol), C₆F₆ (hexafluorobenzene) (135 mL), DMI (N,N′-dimethylimidazolidinone) (91 mL) were added to Sub 1-44c (36.4 g, 90.8 mmol) and 23.6 g of the product (yield: 65.2%) was obtained using the synthetic method of Sub 1-13.

### 3) Synthesis of Sub 2-83c

Sub 2-76a (50.0 g, 157.4 mmol) was dissolved in THF (Tetrahydrofuran) (787 mL) in a round-bottom flask, Sub 2-76b (57.0 g, 157.4 mmol), K₂CO₃ (65.3 g, 472.3 mmol), Pd(PPh₃)₄ (10.91 g, 9.45 mmol), water (394 mL) were added, and 66.2 g of the product (yield: 78.5%) was obtained using the synthetic method of Sub 2-1c.

### 4) Synthesis of Sub 2-83

Sub 2-76c (50.0 g, 93.3 mmol) was dissolved in Toluene (467 mL) in a round-bottom flask, Sub 2-76d (39.2 g, 93.3 mmol), Pd₂(dba)₃ (2.56 g, 2.80 mmol), P(t-Bu)₃ (1.13 g, 5.60 mmol), NaOt-Bu (17.9 g, 186.7 mmol) were added, and 52.7 g of the product (yield: 68.2%) was obtained using the synthetic method of Sub 2-1.

### 5) Synthesis of P-83

Sub 1-44 (10.0 g, 25.0 mmol) was dissolved in Toluene (125 mL) in a round-bottom flask, Sub 2-76 (20.7 g, 25.0 mmol), Pd₂(dba)₃ (0.69 g, 0.75 mmol), P(t-Bu)₃ (0.30 g, 1.50 mmol), NaOt-Bu (4.8 g, 50.1 mmol) were added, and 19.6 g of the product (yield: 68.2%) was obtained using the synthetic method of Sub 2-1.

### 9. Synthesis of P-88

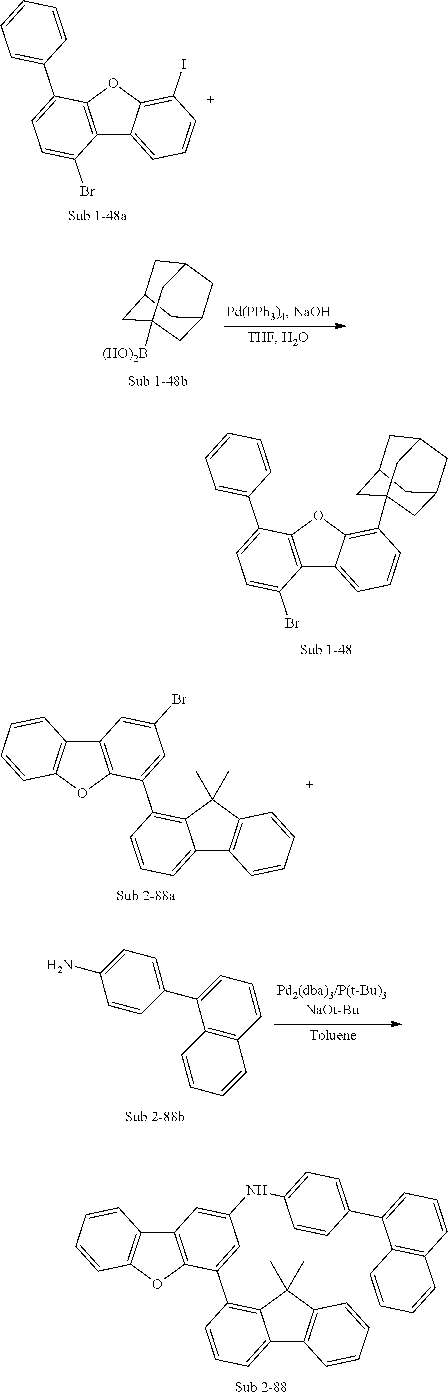 [SMILES: Brc1ccc(-c2ccccc2)c2oc3c(I)cccc3c12 ; OB(O)C12CC3CC(CC(C3)C1)C2 ; Brc1ccc(-c2ccccc2)c2oc3c(C45CC6CC(CC(C6)C4)C5)cccc3c12 ; CC1(C)c2ccccc2-c2cccc(-c3cc(Br)cc4c3oc3ccccc34)c21 ; Nc1ccc(-c2cccc3ccccc23)cc1 ; CC1(C)c2ccccc2-c2cccc(-c3cc(Nc4ccc(-c5cccc6ccccc56)cc4)cc4c3oc3ccccc34)c21]

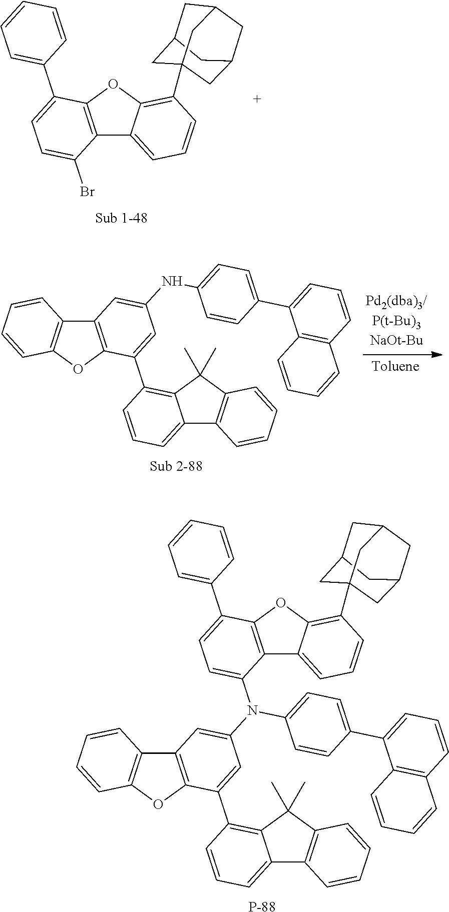

### 1) Synthesis of Sub 1-48

Sub 1-48a (50.0 g, 111.3 mmol) was dissolved in THF (Tetrahydrofuran) (557 mL) in a round-bottom flask, Sub 1-48b (20.0 g, 111.3 mmol), K₂CO₃ (46.2 g, 334.0 mmol), Pd(PPh₃)₄ (7.72 g, 6.68 mmol), water (278 mL) were added, and 40.4 g of the product (yield: 79.3%) was obtained using the synthetic method of Sub 2-1c.

### 2) Synthesis of Sub 2-81

Sub 2-81a (50.0 g, 113.8 mmol) was dissolved in Toluene (569 mL) in a round-bottom flask, Sub 2-81b (25.4 g, 113.8 mmol), Pd₂(dba)₃ (3.13 g, 3.41 mmol), P(t-Bu)₃ (1.38 g, 6.83 mmol), NaOt-Bu (21.9 g, 227.6 mmol) were added, and 44.3 g of the product (yield: 67.4%) was obtained using the synthetic method of Sub 2-1.

### 3) Synthesis of P-88

Sub 1-48 (10 g, 21.9 mmol) was dissolved in Toluene (109 mL) in a round-bottom flask, Sub 2-81 (12.6 g, 21.9 mmol), Pd₂(dba)₃ (0.60 g, 0.66 mmol), P(t-Bu)₃ (0.27 g, 1.31 mmol), NaOt-Bu (4.2 g, 43.7 mmol) were added, and 14.2 g of the product (yield: 68.0%) was obtained using the synthetic method of Sub 2-1.

### 10. Synthesis of P-92

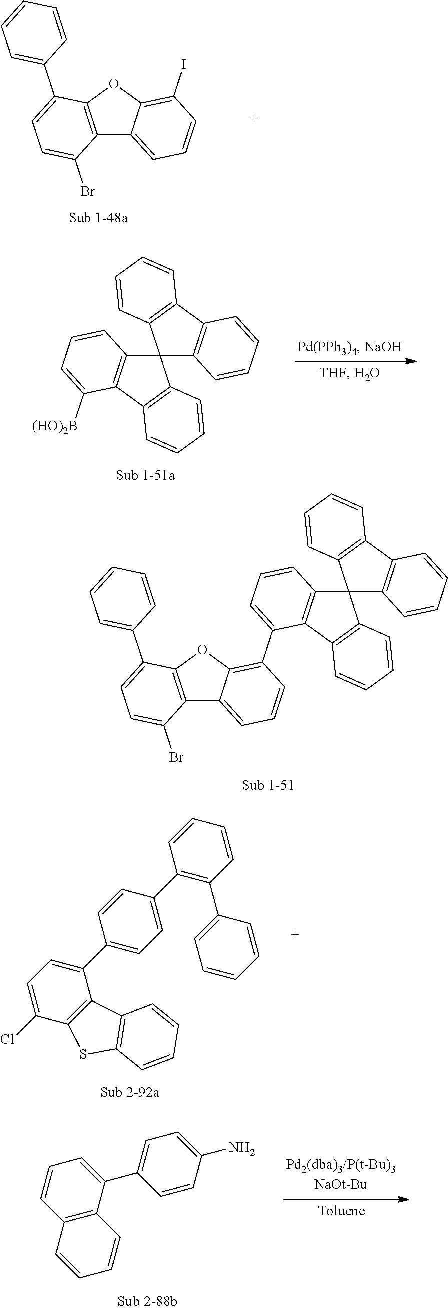 [SMILES: Brc1ccc(-c2ccccc2)c2oc3c(I)cccc3c12 ; OB(O)c1cccc2c1-c1ccccc1C21c2ccccc2-c2ccccc21 ; Brc1ccc(-c2ccccc2)c2oc3c(-c4cccc5c4-c4ccccc4C54c5ccccc5-c5ccccc54)cccc3c12 ; Clc1ccc(-c2ccc(-c3ccccc3-c3ccccc3)cc2)c2c1sc1ccccc12 ; Nc1ccc(-c2cccc3ccccc23)cc1]

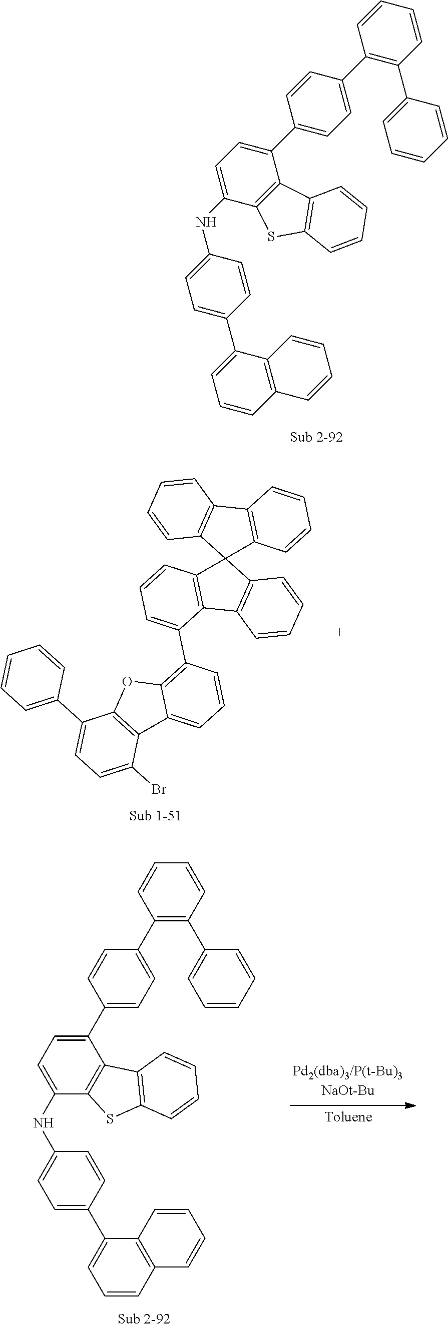 [SMILES: c1ccc(-c2ccccc2-c2ccc(-c3ccc(Nc4ccc(-c5cccc6ccccc56)cc4)c4sc5ccccc5c34)cc2)cc1 ; Brc1ccc(-c2ccccc2)c2oc3c(-c4cccc5c4-c4ccccc4C54c5ccccc5-c5ccccc54)cccc3c12 ; c1ccc(-c2ccccc2-c2ccc(-c3ccc(Nc4ccc(-c5cccc6ccccc56)cc4)c4sc5ccccc5c34)cc2)cc1]

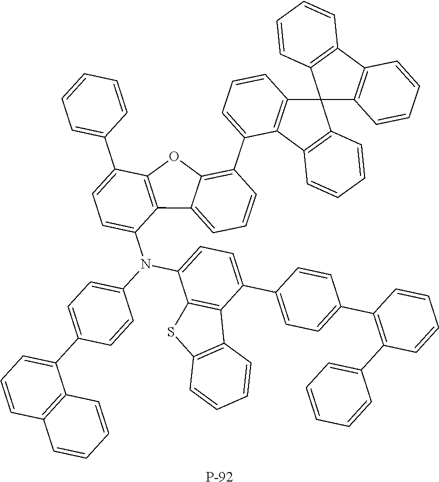

### 1) Synthesis of Sub 1-51

Sub 1-48a (50.0 g, 111.3 mmol) was dissolved in THF (Tetrahydrofuran) (557 mL) in a round-bottom flask, Sub 1-51a (40.1 g, 111.3 mmol), K₂CO₃ (46.2 g, 334.0 mmol), Pd(PPh₃)₄ (7.72 g, 6.68 mmol), water (278 mL) were added, and 56.3 g of the product (yield: 79.3%) was obtained using the synthetic method of Sub 2-1c.

### 2) Synthesis of Sub 2-85

Sub 2-85a (50.0 g, 111.9 mmol) was dissolved in Toluene (559 mL) in a round-bottom flask, Sub 2-81b (24.5 g, 111.9 mmol), Pd₂(dba)₃ (3.07 g, 3.36 mmol), P(t-Bu)₃ (1.36 g, 6.71 mmol), NaOt-Bu (21.5 g, 223.7 mmol) were added, and 48.8 g of the product (yield: 69.2%) was obtained using the synthetic method of Sub 2-1.

### 3) Synthesis of P-92

Sub 1-51 (10 g, 15.7 mmol) was dissolved in Toluene (79 mL) in a round-bottom flask, Sub 2-85 (9.9 g, 15.7 mmol), Pd₂(dba)₃ (0.43 g, 0.47 mmol), P(t-Bu)₃ (0.19 g, 0.94 mmol), NaOt-Bu (3.0 g, 31.4 mmol) were added, and 12.9 g of the product (yield: 69.2%) was obtained using the synthetic method of Sub 2-1.

### 11. Synthesis of P-107

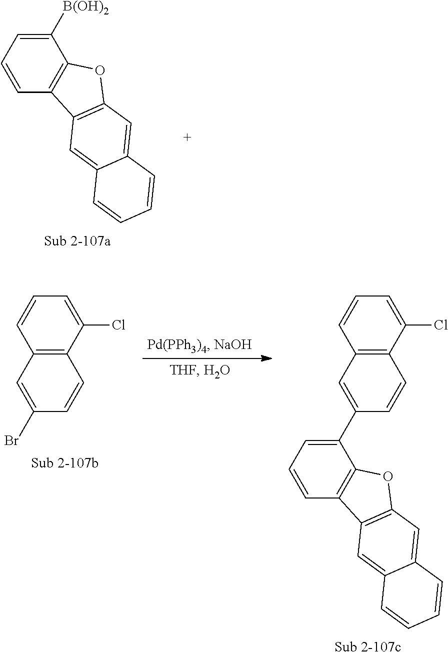

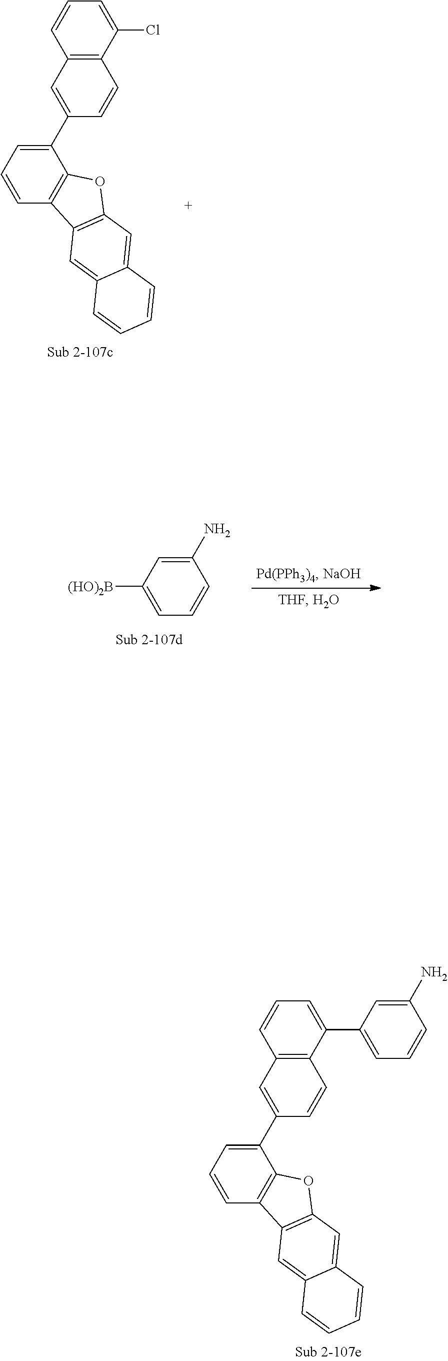

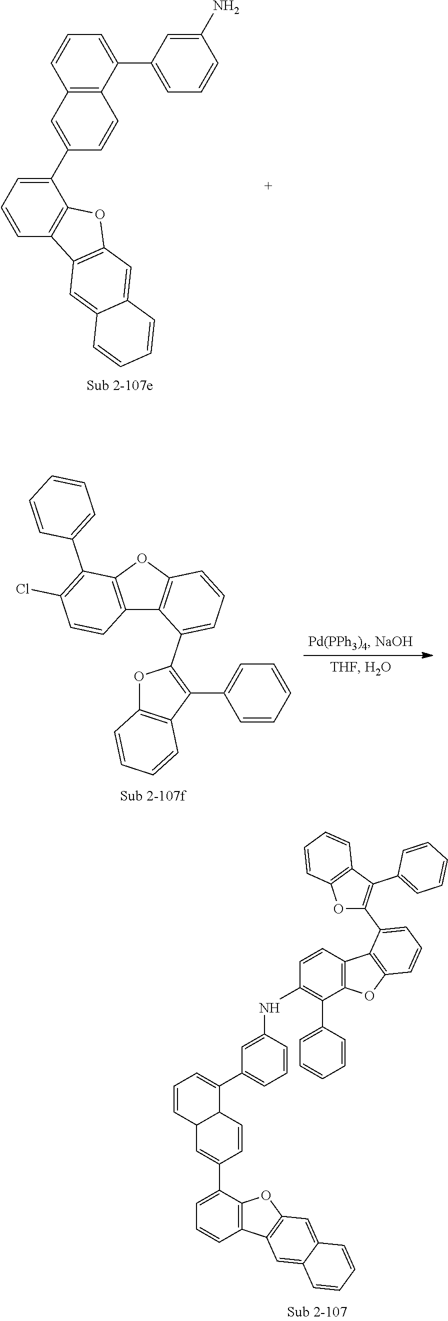

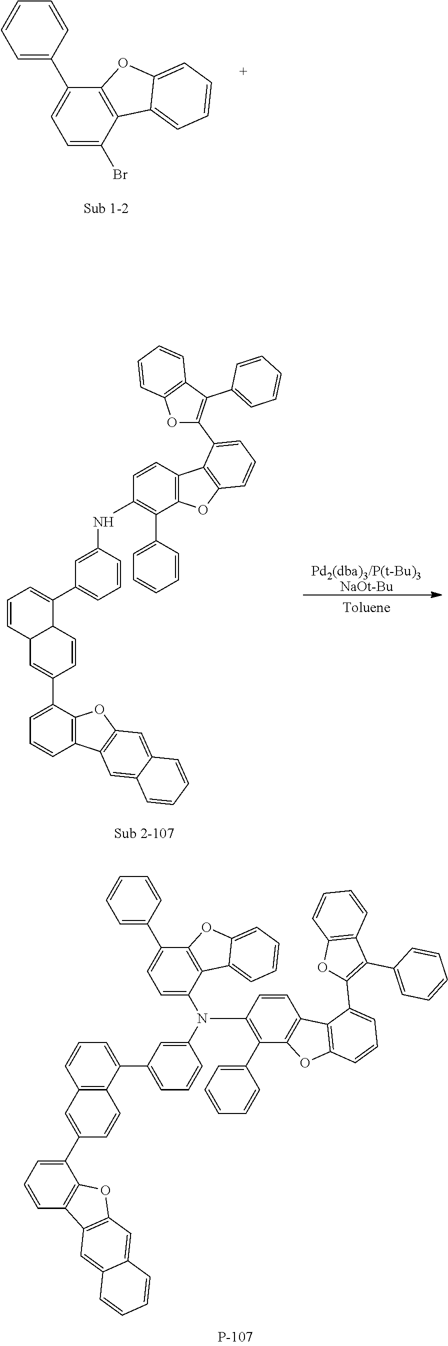 [SMILES: Brc1ccc(-c2ccccc2)c2oc3ccccc3c12 ; C1=CC2C=C(c3cccc4c3oc3cc5ccccc5cc34)C=CC2C(c2cccc(Nc3ccc4c(oc5cccc(-c6oc7ccccc7c6-c6ccccc6)c54)c3-c3ccccc3)c2)=C1 ; "Pd2(dba)3/P(t-Bu)3NaOt-Bu" ; "Toluene" ; c1ccc(-c2c(-c3cccc4oc5c(-c6ccccc6)c(N(c6cccc(-c7cccc8cc(-c9cccc%10c9oc9cc%11ccccc%11cc9%10)ccc78)c6)c6ccc(-c7ccccc7)c7oc8ccccc8c67)ccc5c34)oc3ccccc23)cc1]

### 1) Synthesis of Sub 2-100c

Sub 2-100a (70.0 g, 267.1 mmol) was dissolved in THF (Tetrahydrofuran) (1336 mL) in a round-bottom flask, Sub 2-100b (64.5 g, 267.1 mmol), K₂CO₃ (110.7 g, 801.3 mmol), Pd(PPh₃)₄ (18.52 g, 16.03 mmol), water (668 mL) were added, and 78.7 g of the product (yield: 77.8%) was obtained using the synthetic method of Sub 2-1c.

### 2) Synthesis of Sub 2-100e

Sub 2-100c (70.0 g, 184.8 mmol) was dissolved in THF (Tetrahydrofuran) (924 mL) in a round-bottom flask, Sub 2-100d (25.3 g, 184.8 mmol), K₂CO₃ (76.6 g, 554.3 mmol), Pd(PPh₃)₄ (12.81 g, 11.09 mmol), water (462 mL) were added, and 64.4 g of the product (yield: 80.0%) was obtained using the synthetic method of Sub 2-1c.

### 3) Synthesis of Sub 2-100

Sub 2-100e (50.0 g, 114.8 mmol) was dissolved in Toluene (574 mL) in a round-bottom flask, Sub 2-100f (54.1 g, 114.8 mmol), Pd₂(dba)₃ (3.15 g, 3.44 mmol), P(t-Bu)₃ (1.39 g, 6.89 mmol), NaOt-Bu (22.1 g, 229.6 mmol) were added, and 70.3 g of the product (yield: 70.2%) was obtained using the synthetic method of Sub 2-1.

### 4) Synthesis of P-107

Sub 1-2 (10 g, 30.9 mmol) was dissolved in Toluene (155 mL) in a round-bottom flask, Sub 2-100 (27.0 g, 30.9 mmol), Pd₂(dba)₃ (0.85 g, 0.93 mmol), P(t-Bu)₃ (0.38 g, 1.86 mmol), NaOt-Bu (5.9 g, 61.9 mmol) were added, and 23.3 g of the product (yield: 67.6%) was obtained using the synthetic method of Sub 2-1.

### 12. Synthesis of P-108

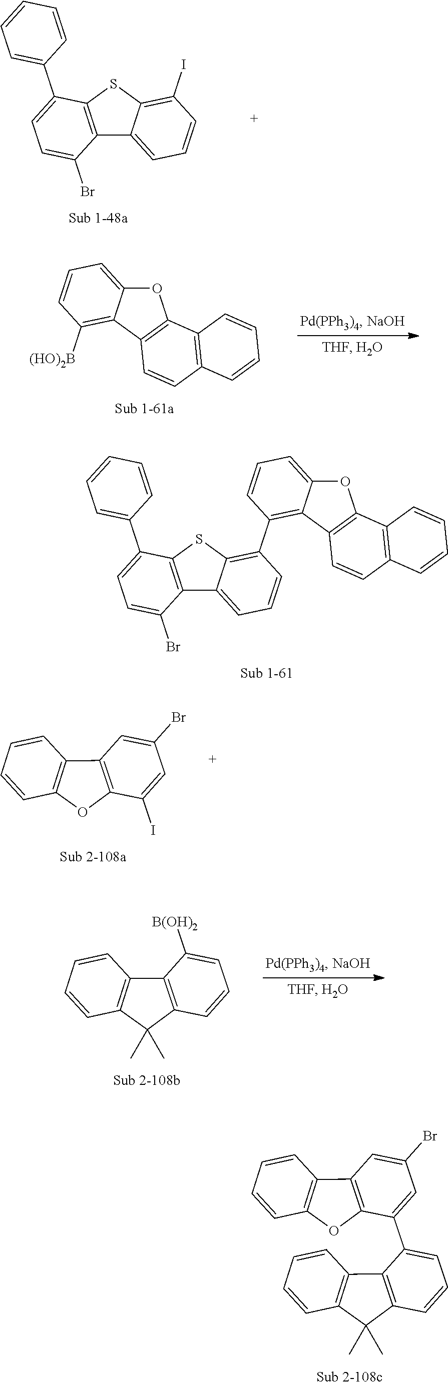 [SMILES: Brc1ccc(-c2ccccc2)c2sc3c(I)cccc3c12 ; OB(O)c1cccc2oc3c4ccccc4ccc3c12 ; Brc1ccc(-c2ccccc2)c2sc3c(-c4cccc5oc6c7ccccc7ccc6c45)cccc3c12 ; Brc1cc(I)c2oc3ccccc3c2c1 ; CC1(C)c2ccccc2-c2c(B(O)O)cccc21 ; CC1(C)c2ccccc2-c2c(-c3cc(Br)cc4c3oc3ccccc34)cccc21]

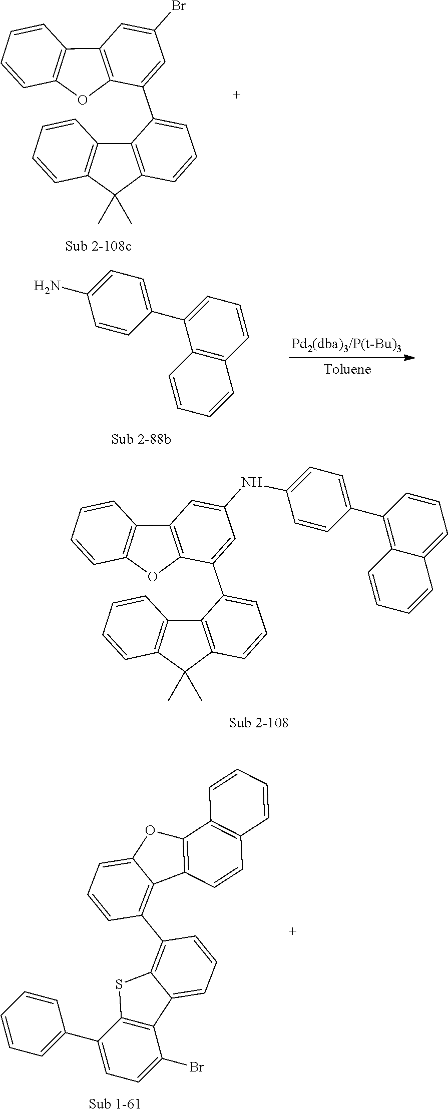 [SMILES: CC1(C)c2ccccc2-c2c(-c3cc(Br)cc4c3oc3ccccc34)cccc21 ; Nc1ccc(-c2cccc3ccccc23)cc1 ; CC1(C)c2ccccc2-c2c(-c3cc(Nc4ccc(-c5cccc6ccccc56)cc4)cc4c3oc3ccccc34)cccc21 ; Brc1ccc(-c2ccccc2)c2sc3c(-c4cccc5oc6c7ccccc7ccc6c45)cccc3c12]

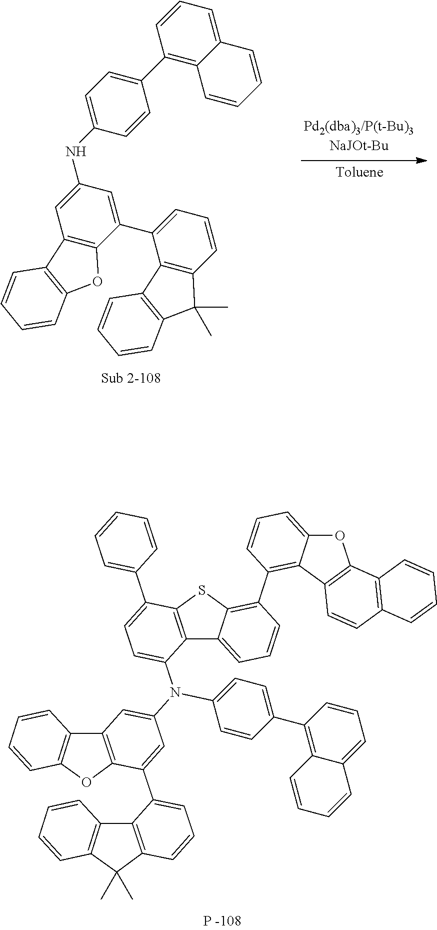 [SMILES: CC1(C)c2ccccc2-c2c(-c3cc(Nc4ccc(-c5cccc6ccccc56)cc4)cc4c3oc3ccccc34)cccc21 ; CC1(C)c2ccccc2-c2c(-c3cc(N(c4ccc(-c5cccc6ccccc56)cc4)c4ccc(-c5ccccc5)c5sc6c(-c7cccc8oc9c%10ccccc%10ccc9c78)cccc6c45)cc4c3oc3ccccc34)cccc21]

### 1) Synthesis of Sub 1-61

Sub 1-48a (50.0 g, 107.5 mmol) was dissolved in THF (Tetrahydrofuran) (537 mL) in a round-bottom flask, Sub 1-61a (28.2 g, 262.07 mmol), K₂CO₃ (44.6 g, 322.5 mmol), Pd(PPh₃)₄ (7.45 g, 6.45 mmol), water (269 mL) were added, and 45.3 g of the product (yield: 75.8%) was obtained using the synthetic method of Sub 2-1c.

### 2) Synthesis of Sub 2-101c

Sub 2-101a (50.0 g, 134.1 mmol) was dissolved in THF (Tetrahydrofuran) (670 mL) in a round-bottom flask, Sub 2-101b (31.9 g, 134.1 mmol), K₂CO₃ (55.6 g, 402.2 mmol), Pd(PPh₃)₄ (9.29 g, 8.04 mmol), water (335 mL) were added, and 46.5 g of the product (yield: 78.9%) was obtained using the synthetic method of Sub 2-1c.

### 3) Synthesis of Sub 2-101

Sub 2-101c (40.0 g, 91.0 mmol) was dissolved in Toluene (455 mL) in a round-bottom flask, Sub 2-81b (20.0 g, 91.0 mmol), Pd₂(dba)₃ (2.50 g, 2.73 mmol), P(t-Bu)₃ (1.11 g, 5.46 mmol), NaOt-Bu (17.5 g, 182.1 mmol) were added, and 36.6 g of the product (yield: 69.5%) was obtained using the synthetic method of Sub 2-1.

### 4) Synthesis of P-108

Sub 1-61 (10 g, 18.0 mmol) was dissolved in Toluene (90 mL) in a round-bottom flask, Sub 2-101 (10.4 g, 18.0 mmol), Pd₂(dba)₃ (0.49 g, 0.54 mmol), P(t-Bu)₃ (0.22 g, 1.08 mmol), NaOt-Bu (3.5 g, 36.0 mmol) were added, and 12.9 g of the product (yield: 67.9%) was obtained using the synthetic method of Sub 2-1.

Sub 1 of Reaction Scheme 1 may be a compound as follows, but is not limited thereto, and the FD-MS (Field Desorption-Mass Spectrometry) values of compounds belonging to the following Sub 1 are as shown in Table 1.

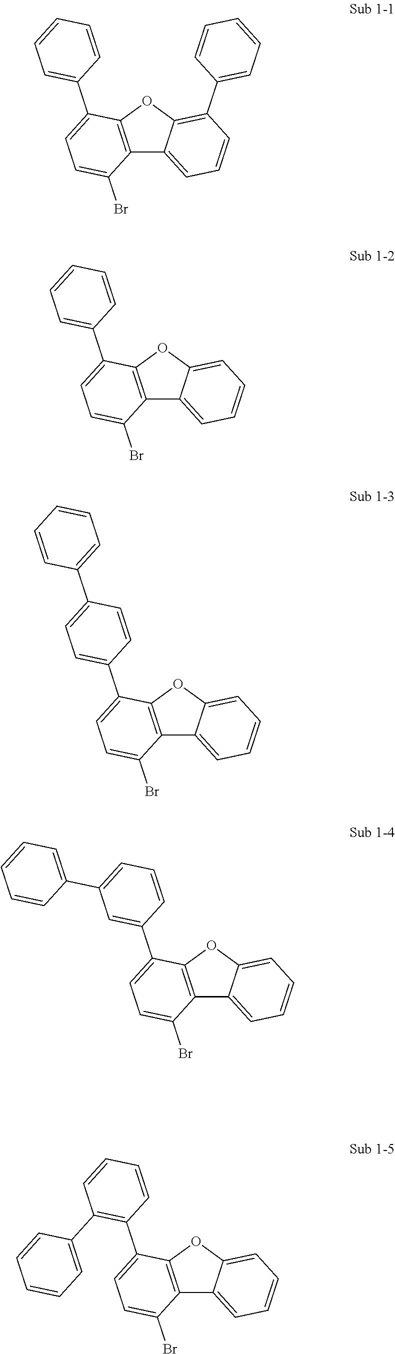 [SMILES: Brc1ccc(-c2ccccc2)c2oc3c(-c4ccccc4)cccc3c12 ; Brc1ccc(-c2ccccc2)c2oc3ccccc3c12 ; Brc1ccc(-c2ccc(-c3ccccc3)cc2)c2oc3ccccc3c12 ; Brc1ccc(-c2cccc(-c3ccccc3)c2)c2oc3ccccc3c12 ; Brc1ccc(-c2ccccc2-c2ccccc2)c2oc3ccccc3c12]

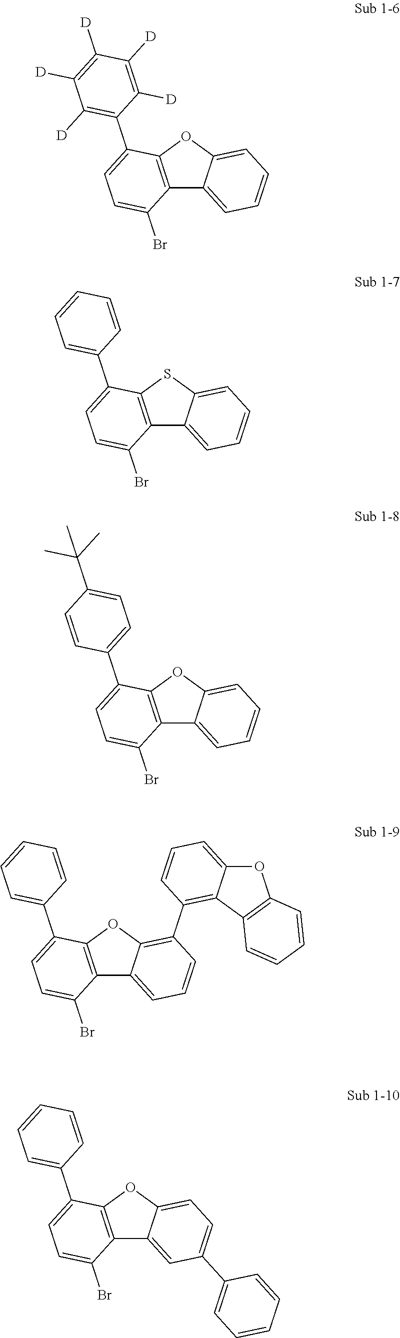 [SMILES: [2H]c1c([2H])c([2H])c(-c2ccc(Br)c3c2oc2ccccc23)c([2H])c1[2H] ; Brc1ccc(-c2ccccc2)c2sc3ccccc3c12 ; CC(C)(C)c1ccc(-c2ccc(Br)c3c2oc2ccccc23)cc1 ; Brc1ccc(-c2ccccc2)c2oc3c(-c4cccc5oc6ccccc6c45)cccc3c12 ; Brc1ccc(-c2ccccc2)c2oc3ccc(-c4ccccc4)cc3c12]

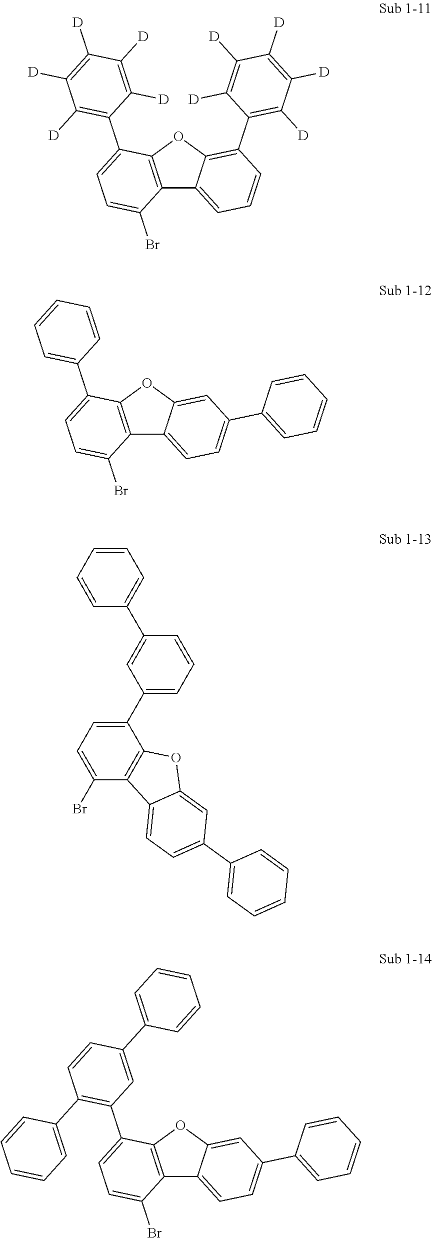 [SMILES: [2H]c1c([2H])c([2H])c(-c2cccc3c2oc2c(-c4c([2H])c([2H])c([2H])c([2H])c4[2H])ccc(Br)c23)c([2H])c1[2H] ; Brc1ccc(-c2ccccc2)c2oc3cc(-c4ccccc4)ccc3c12 ; Brc1ccc(-c2cccc(-c3ccccc3)c2)c2oc3cc(-c4ccccc4)ccc3c12 ; Brc1ccc(-c2cc(-c3ccccc3)ccc2-c2ccccc2)c2oc3cc(-c4ccccc4)ccc3c12]

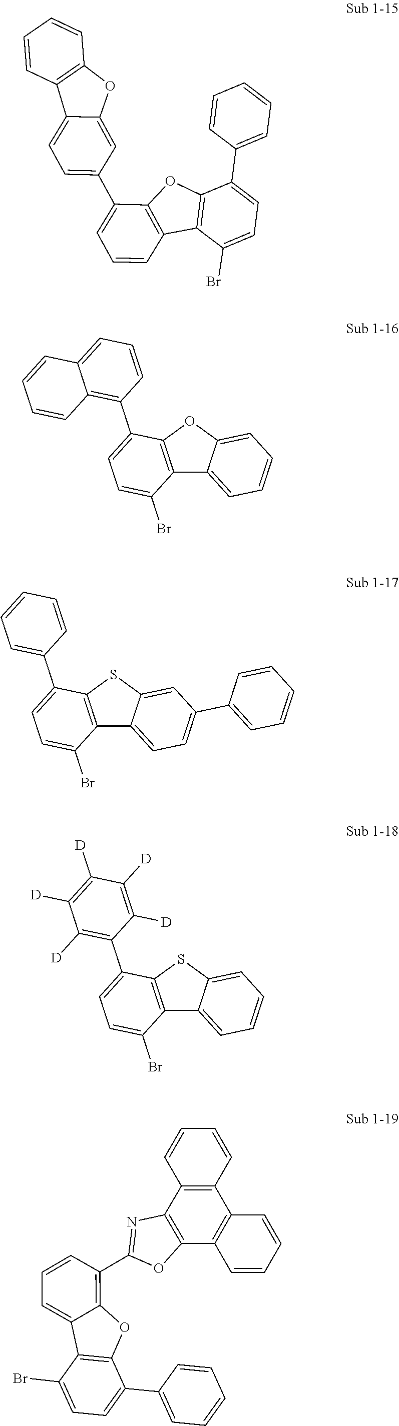 [SMILES: Brc1ccc(-c2ccccc2)c2oc3c(-c4ccc5c(c4)oc4ccccc45)cccc3c12 ; Brc1ccc(-c2cccc3ccccc23)c2oc3ccccc3c12 ; Brc1ccc(-c2ccccc2)c2sc3cc(-c4ccccc4)ccc3c12 ; [2H]c1c([2H])c([2H])c(-c2ccc(Br)c3c2sc2ccccc23)c([2H])c1[2H] ; Brc1ccc(-c2ccccc2)c2oc3c(-c4nc5c6ccccc6c6ccccc6c5o4)cccc3c12]

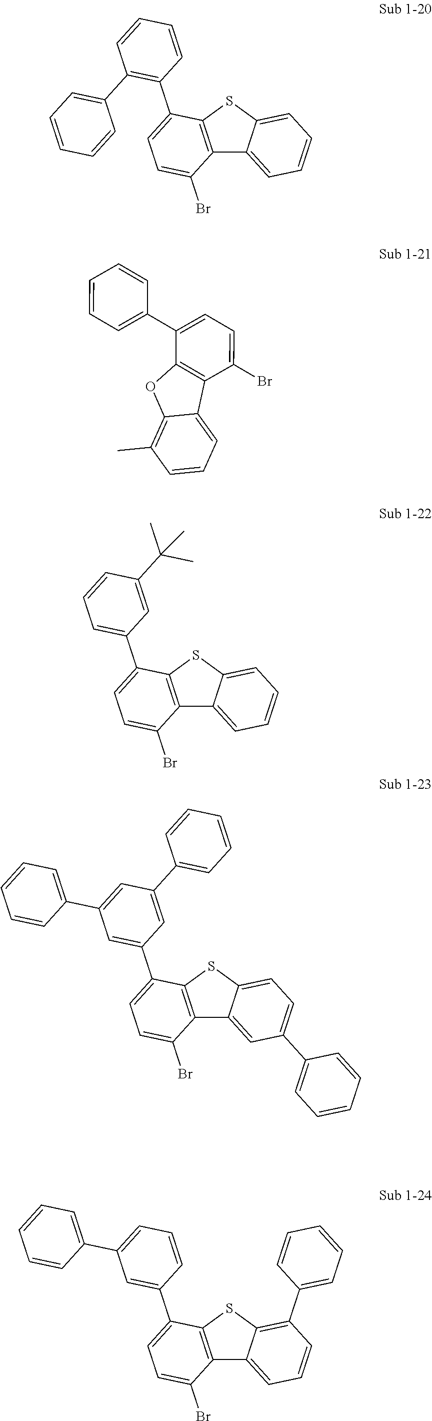 [SMILES: Brc1ccc(-c2ccccc2-c2ccccc2)c2sc3ccccc3c12 ; Cc1cccc2c1oc1c(-c3ccccc3)ccc(Br)c12 ; CC(C)(C)c1cccc(-c2ccc(Br)c3c2sc2ccccc23)c1 ; Brc1ccc(-c2cc(-c3ccccc3)cc(-c3ccccc3)c2)c2sc3ccc(-c4ccccc4)cc3c12 ; Brc1ccc(-c2cccc(-c3ccccc3)c2)c2sc3c(-c4ccccc4)cccc3c12]

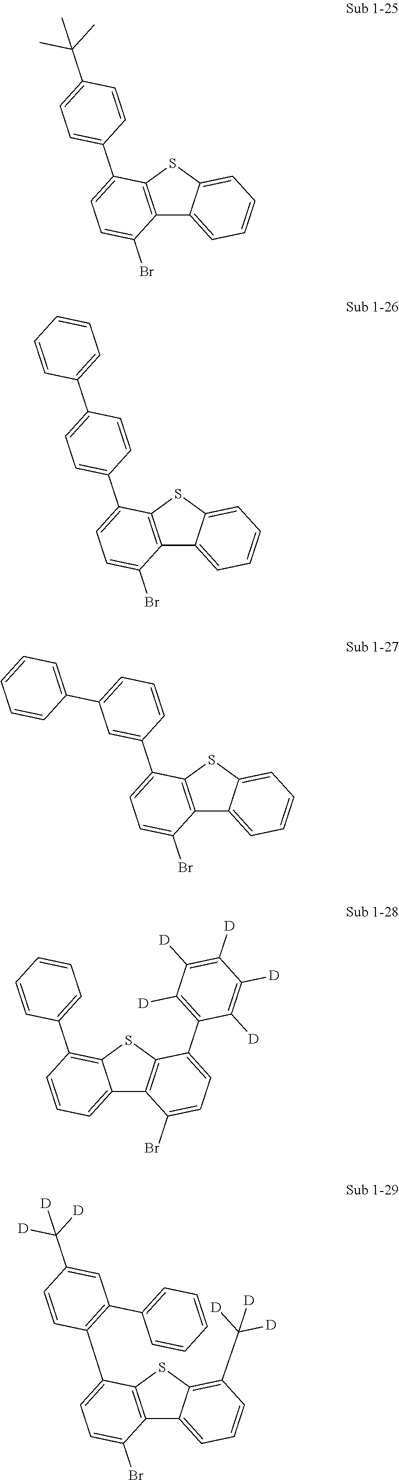 [SMILES: CC(C)(C)c1ccc(-c2ccc(Br)c3c2sc2ccccc23)cc1 ; Brc1ccc(-c2ccc(-c3ccccc3)cc2)c2sc3ccccc3c12 ; Brc1ccc(-c2cccc(-c3ccccc3)c2)c2sc3ccccc3c12 ; [2H]c1c([2H])c([2H])c(-c2ccc(Br)c3c2sc2c(-c4ccccc4)cccc23)c([2H])c1[2H] ; [2H]C([2H])([2H])c1ccc(-c2ccc(Br)c3c2sc2c(C([2H])([2H])[2H])cccc23)c(-c2ccccc2)c1]

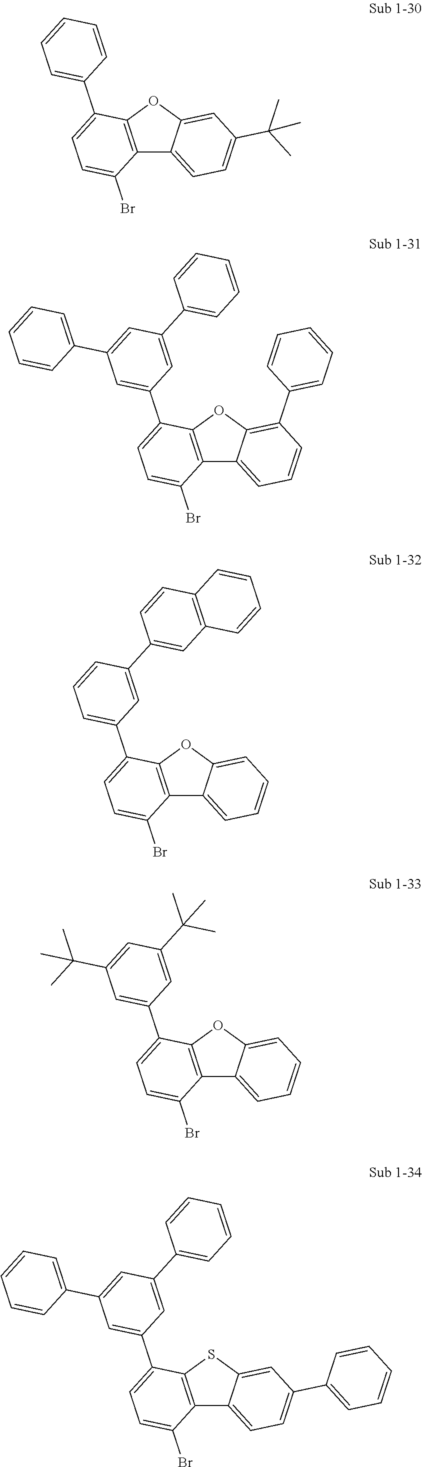

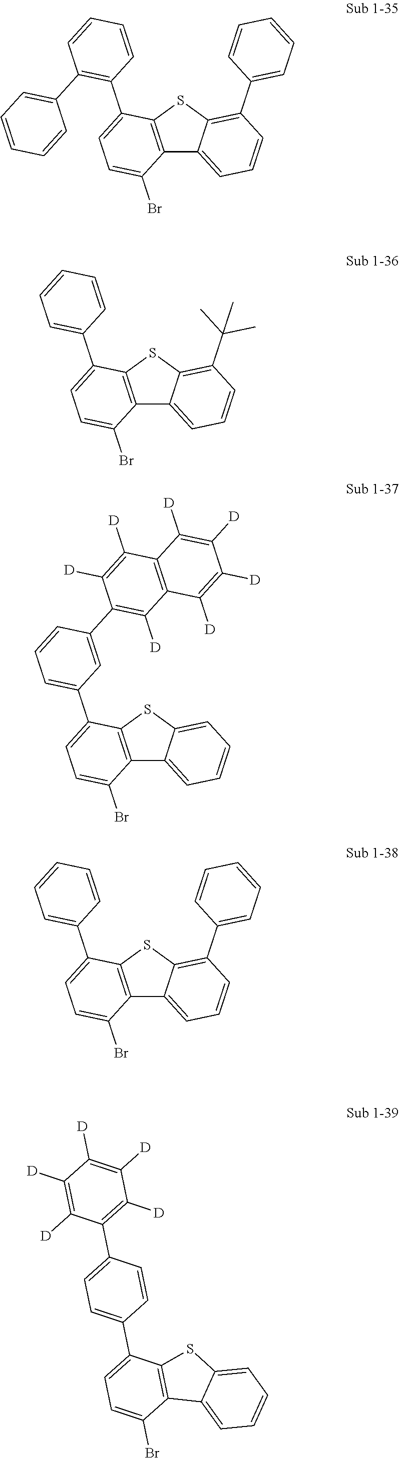 [SMILES: Brc1ccc(-c2ccccc2-c2ccccc2)c2sc3c(-c4ccccc4)cccc3c12 ; CC(C)(C)c1cccc2c1sc1c(-c3ccccc3)ccc(Br)c12 ; [2H]c1c([2H])c([2H])c2c([2H])c(-c3cccc(-c4ccc(Br)c5c4sc4ccccc45)c3)c([2H])c([2H])c2c1[2H] ; Brc1ccc(-c2ccccc2)c2sc3c(-c4ccccc4)cccc3c12 ; [2H]c1c([2H])c([2H])c(-c2ccc(-c3ccc(Br)c4c3sc3ccccc34)cc2)c([2H])c1[2H]]

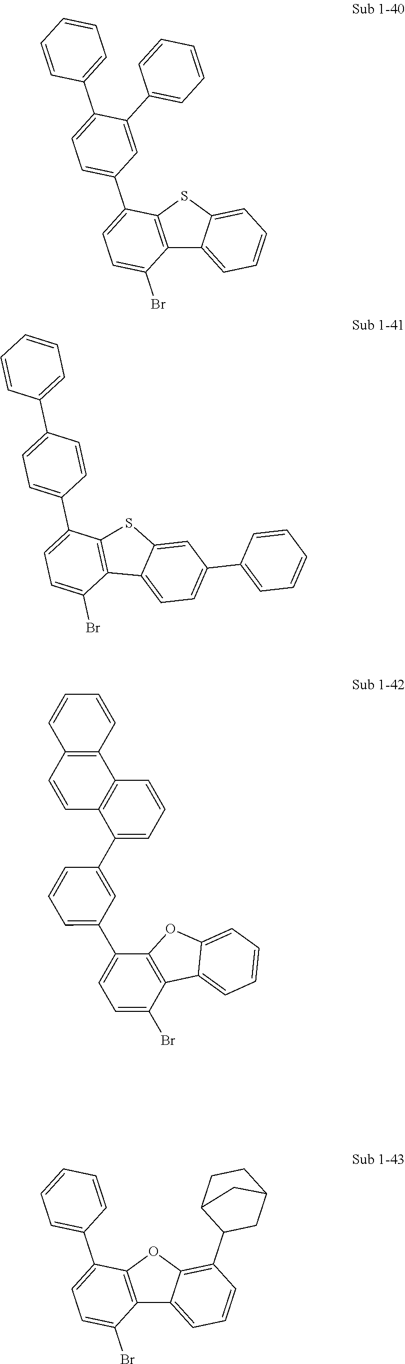 [SMILES: Brc1ccc(-c2ccc(-c3ccccc3)c(-c3ccccc3)c2)c2sc3ccccc3c12 ; Brc1ccc(-c2ccc(-c3ccccc3)cc2)c2sc3cc(-c4ccccc4)ccc3c12 ; Brc1ccc(-c2cccc(-c3cccc4c3ccc3ccccc34)c2)c2oc3ccccc3c12 ; Brc1ccc(-c2ccccc2)c2oc3c(C4CC5CCC4C5)cccc3c12]

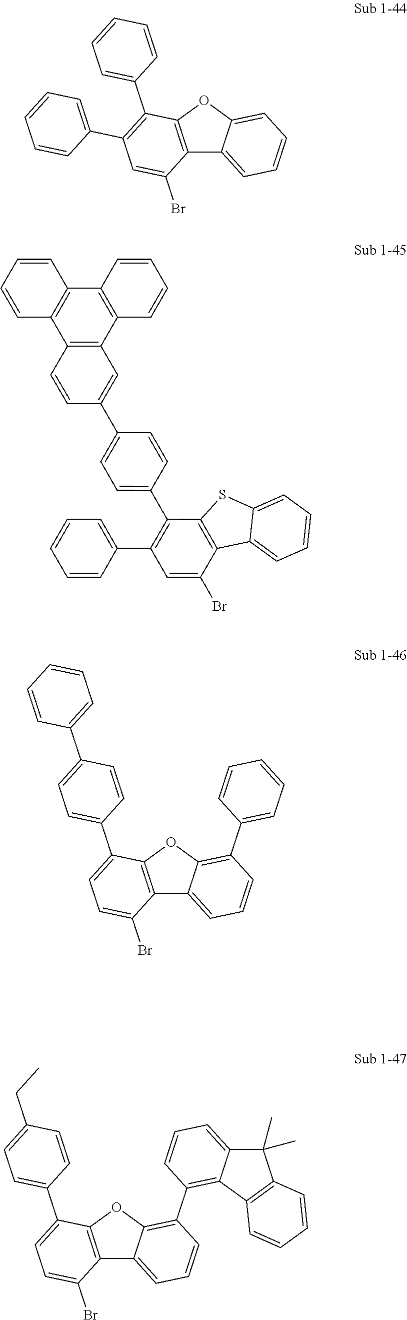 [SMILES: Brc1cc(-c2ccccc2)c(-c2ccccc2)c2oc3ccccc3c12 ; Brc1cc(-c2ccccc2)c(-c2ccc(-c3ccc4c5ccccc5c5ccccc5c4c3)cc2)c2sc3ccccc3c12 ; Brc1ccc(-c2ccc(-c3ccccc3)cc2)c2oc3c(-c4ccccc4)cccc3c12 ; CCc1ccc(-c2ccc(Br)c3c2oc2c(-c4cccc5c4-c4ccccc4C5(C)C)cccc23)cc1]

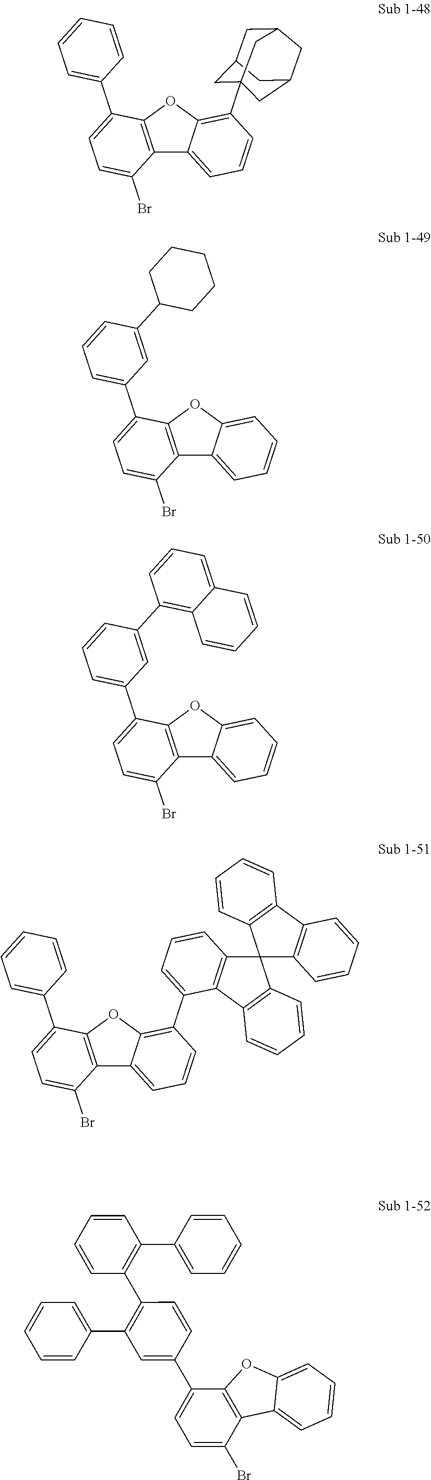

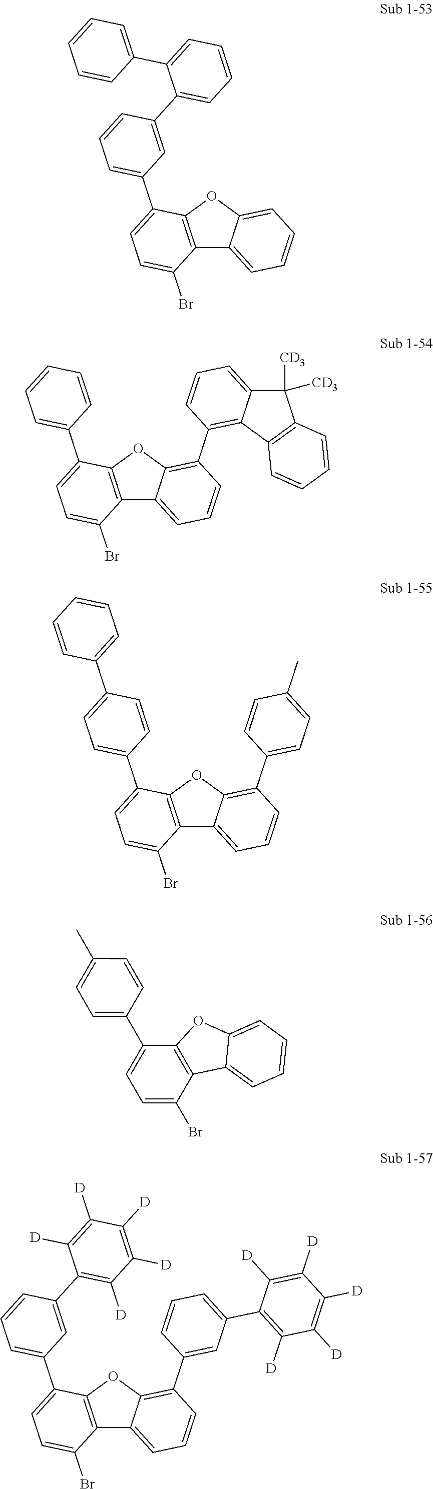 [SMILES: Brc1ccc(-c2cccc(-c3ccccc3-c3ccccc3)c2)c2oc3ccccc3c12 ; [2H]C([2H])([2H])C1(C([2H])([2H])[2H])c2ccccc2-c2c(-c3cccc4c3oc3c(-c5ccccc5)ccc(Br)c34)cccc21 ; Cc1ccc(-c2cccc3c2oc2c(-c4ccc(-c5ccccc5)cc4)ccc(Br)c23)cc1 ; Cc1ccc(-c2ccc(Br)c3c2oc2ccccc23)cc1 ; [2H]c1c([2H])c([2H])c(-c2cccc(-c3cccc4c3oc3c(-c5cccc(-c6c([2H])c([2H])c([2H])c([2H])c6[2H])c5)ccc(Br)c34)c2)c([2H])c1[2H]]

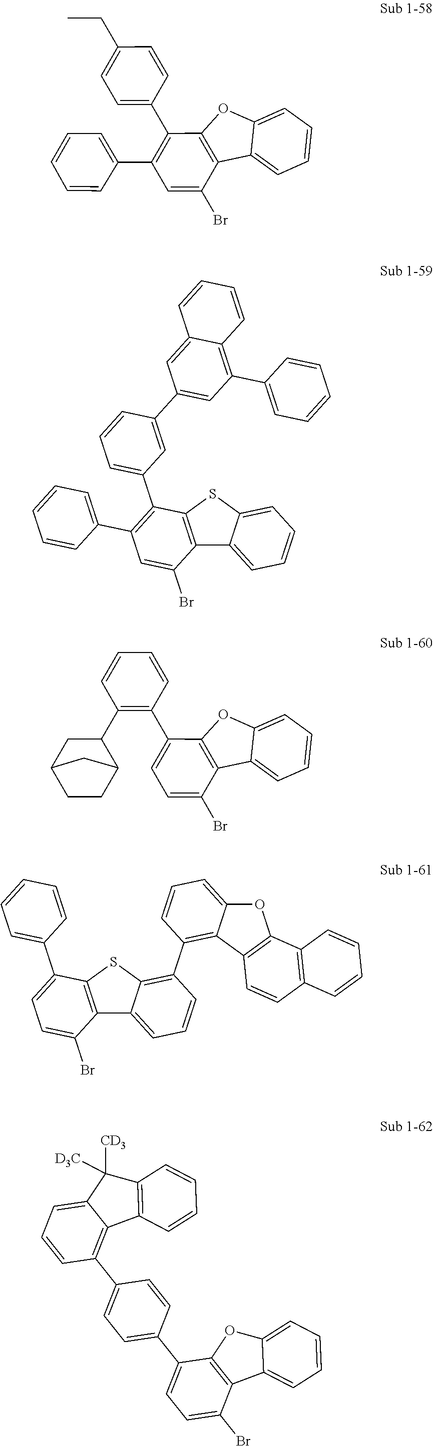 [SMILES: CCc1ccc(-c2c(-c3ccccc3)cc(Br)c3c2oc2ccccc23)cc1 ; Brc1cc(-c2ccccc2)c(-c2cccc(-c3cc(-c4ccccc4)c4ccccc4c3)c2)c2sc3ccccc3c12 ; Brc1ccc(-c2ccccc2C2CC3CCC2C3)c2oc3ccccc3c12 ; Brc1ccc(-c2ccccc2)c2sc3c(-c4cccc5oc6c7ccccc7ccc6c45)cccc3c12 ; [2H]C([2H])([2H])C1(C([2H])([2H])[2H])c2ccccc2-c2c(-c3ccc(-c4ccc(Br)c5c4oc4ccccc45)cc3)cccc21]

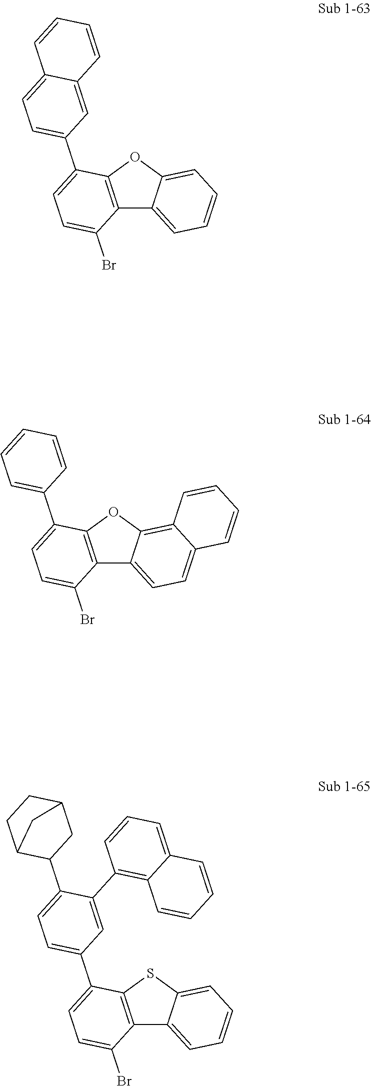 [SMILES: Brc1ccc(-c2ccc3ccccc3c2)c2oc3ccccc3c12 ; Brc1ccc(-c2ccccc2)c2oc3c4ccccc4ccc3c12 ; Brc1ccc(-c2ccc(C3CC4CCC3C4)c(-c3cccc4ccccc34)c2)c2sc3ccccc3c12]

TABLE 1

| Compound | FD-MS | Compound | FD-MS |
|---|---|---|---|
| Sub 1-1 | m/z = 398.03(C₂₄H₁₅BrO = 399.29) | Sub 1-2 | m/z = 322(C₁₈H₁₁BrO = 323.19) |
| Sub 1-3 | m/z = 398.03(C₂₄H₁₅BrO = 399.29) | Sub 1-4 | m/z = 398.03(C₂₄H₁₅BrO = 399.29) |
| Sub 1-5 | m/z = 398.03(C₂₄H₁₅BrO = 399.29) | Sub 1-6 | m/z = 327.03(C₁₈H₆D₅BrO = 328.22) |
| Sub 1-7 | m/z = 337.98(C₁₈H₁₁BrS = 339.25) | Sub 1-8 | m/z = 378.06(C₂₂H₁₉BrO = 379.3) |
| Sub 1-9 | m/z = 488.04(C₃₀H₁₇BrO₂ = 489.37) | Sub 1-10 | m/z = 398.03(C₂₄H₁₅BrO = 399.29) |
| Sub 1-11 | m/z = 408.09(C₂₄H₅D₁₀BrO = 409.35) | Sub 1-12 | m/z = 398.03(C₂₄H₁₅BrO = 399.29) |
| Sub 1-13 | m/z = 474.06(C₃₀H₁₉BrO = 475.39) | Sub 1-14 | m/z = 550.09(C₃₆H₂₃BrO = 551.48) |
| Sub 1-15 | m/z = 488.04(C₃₀H₁₇BrO₂ = 489.37) | Sub 1-16 | m/z = 372.01(C₂₂H₁₃BrO = 373.25) |
| Sub 1-17 | m/z = 414.01(C₂₄H₁₅BrS = 415.35) | Sub 1-18 | m/z = 343.01(C₁₈H₆D₅BrS = 344.28) |
| Sub 1-19 | m/z = 539.05(C₃₃H₁₈BrNO₂ = 540.42) | Sub 1-20 | m/z = 414.01(C₂₄H₁₅BrS = 415.35) |
| Sub 1-21 | m/z = 336.01(C₁₉H₁₃BrO = 337.22) | Sub 1-22 | m/z = 394.04(C₂₂H₁₉BrS = 395.36) |
| Sub 1-23 | m/z = 566.07(C₃₆H₂₃BrS = 567.54) | Sub 1-24 | m/z = 490.04(C₃₀H₁₉BrS = 491.45) |
| Sub 1-25 | m/z = 394.04(C₂₂H₁₉BrS = 395.36) | Sub 1-26 | m/z = 414.01(C₂₄H₁₅BrS = 415.35) |
| Sub 1-27 | m/z = 414.01(C₂₄H₁₅BrS = 415.35) | Sub 1-28 | m/z = 419.04(C₂₄H₁₀D₅BrS = 420.38) |
| Sub 1-29 | m/z = 448.08(C₂₆H₁₃D₆BrS = 449.44) | Sub 1-30 | m/z = 378.06(C₂₂H₁₉BrO = 379.3) |
| Sub 1-31 | m/z = 550.09(C₃₆H₂₃BrO = 551.48) | Sub 1-32 | m/z = 448.05(C₂₈H₁₇BrO = 449.35) |
| Sub 1-33 | m/z = 434.12(C₂₆H₂₇BrO = 435.41) | Sub 1-34 | m/z = 566.07(C₃₆H₂₃BrS = 567.54) |
| Sub 1-35 | m/z = 490.04(C₃₀H₁₉BrS = 491.45) | Sub 1-36 | m/z = 394.04(C₂₂H₁₉BrS = 395.36) |
| Sub 1-37 | m/z = 471.07(C₂₈H₁₀D₇BrS = 472.45) | Sub 1-38 | m/z = 414.01(C₂₄H₁₅BrS = 415.35) |
| Sub 1-39 | m/z = 419.04(C₂₄H₁₀D₅BrS = 420.38) | Sub 1-40 | m/z = 490.04(C₃₀H₁₉BrS = 491.45) |
| Sub 1-41 | m/z = 490.04(C₃₀H₁₉BrS = 491.45) | Sub 1-42 | m/z = 498.06(C₃₂H₁₉BrO = 499.41) |
| Sub 1-43 | m/z = 416.08(C₂₅H₂₁BrO = 417.35) | Sub 1-44 | m/z = 398.03(C₂₄H₁₅BrO = 399.29) |
| Sub 1-45 | m/z = 640.09(C₄₂H₂₅BrS = 641.63) | Sub 1-46 | m/z = 474.06(C₃₀H₁₉BrO = 475.39) |
| Sub 1-47 | m/z = 542.12(C₃₅H₂₇BrO = 543.5) | Sub 1-48 | m/z = 456.11(C₂₈H₂₅BrO = 457.41) |
| Sub 1-49 | m/z = 404.08(C₂₄H₂₁BrO = 405.34) | Sub 1-50 | m/z = 448.05(C₂₈H₁₇BrO = 449.35) |
| Sub 1-51 | m/z = 636.11(C₄₃H₂₅BrO = 637.58) | Sub 1-52 | m/z = 550.09(C₃₆H₂₃BrO = 551.48) |
| Sub 1-53 | m/z = 474.06(C₃₀H₁₉BrO = 475.39) | Sub 1-54 | m/z = 520.13(C₃₃H₁₇D₆BrO = 521.49) |
| Sub 1-55 | m/z = 488.08(C₃₁H₂₁BrO = 489.41) | Sub 1-56 | m/z = 336.01(C₁₉H₁₃BrO = 337.22) |
| Sub 1-57 | m/z = 560.16(C₃₆H₁₃D₁₀BrO = 561.54) | Sub 1-58 | m/z = 426.06(C₂₆H₁₉BrO = 427.34) |
| Sub 1-59 | m/z = 616.09(C₄₀H₂₅BrS = 617.6) | Sub 1-60 | m/z = 416.08(C₂₅H₂₁BrO = 417.35) |
| Sub 1-61 | m/z = 554.03(C₃₄H₁₉BrOS = 555.49) | Sub 1-62 | m/z = 520.13(C₃₃H₁₇D₆BrO = 521.49) |
| Sub 1-63 | m/z = 372.01(C₂₂H₁₃BrO = 373.25) | Sub 1-64 | m/z = 372.01(C₂₂H₁₃BrO = 373.25) |
| Sub 1-65 | m/z = 558.1(C₃₅H₂₇BrS = 559.57) | | |

Sub 2 of Reaction Scheme 1 may be a compound as follows, but is not limited thereto, and the FD-MS (Field Desorption-Mass Spectrometry) values of compounds belonging to Sub 2 are as shown in Table 2.

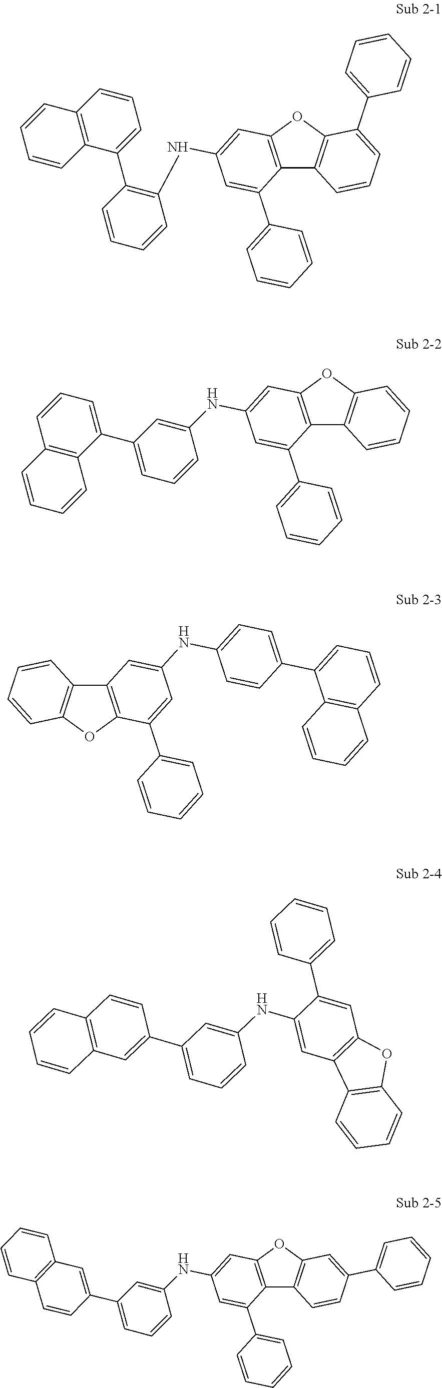 [SMILES: c1ccc(-c2cccc3c2oc2cc(Nc4ccccc4-c4cccc5ccccc45)cc(-c4ccccc4)c23)cc1 ; c1ccc(-c2cc(Nc3cccc(-c4cccc5ccccc45)c3)cc3oc4ccccc4c23)cc1 ; c1ccc(-c2cc(Nc3ccc(-c4cccc5ccccc45)cc3)cc3c2oc2ccccc23)cc1 ; c1ccc(-c2cc3oc4ccccc4c3cc2Nc2cccc(-c3ccc4ccccc4c3)c2)cc1 ; c1ccc(-c2ccc3c(c2)oc2cc(Nc4cccc(-c5ccc6ccccc6c5)c4)cc(-c4ccccc4)c23)cc1]

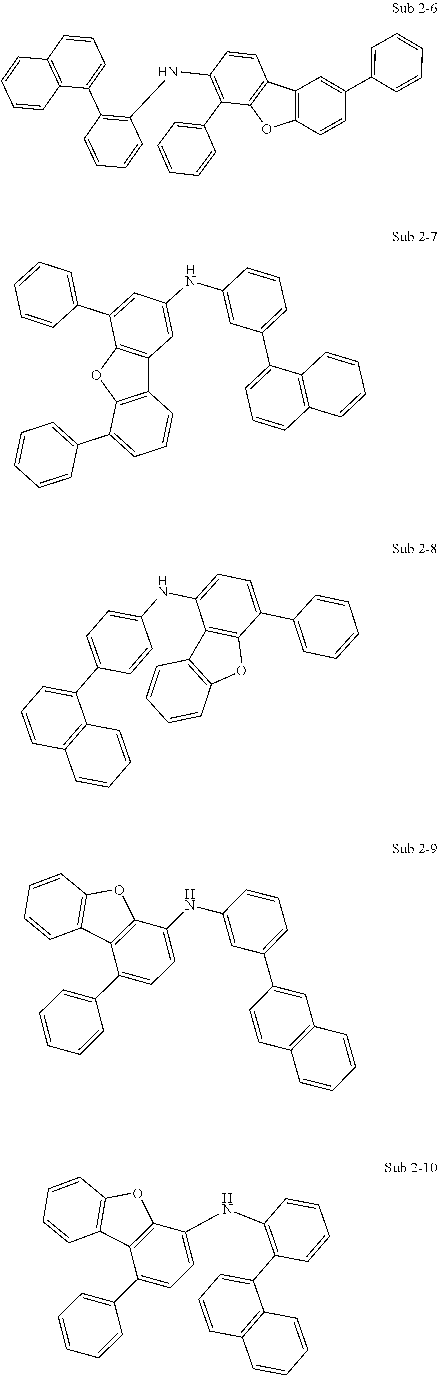 [SMILES: c1ccc(-c2ccc3oc4c(-c5ccccc5)c(Nc5ccccc5-c5cccc6ccccc56)ccc4c3c2)cc1 ; c1ccc(-c2cccc3c2oc2c(-c4ccccc4)cc(Nc4cccc(-c5cccc6ccccc56)c4)cc23)cc1 ; c1ccc(-c2ccc(Nc3ccc(-c4cccc5ccccc45)cc3)c3c2oc2ccccc23)cc1 ; c1ccc(-c2ccc(Nc3cccc(-c4ccc5ccccc5c4)c3)c3oc4ccccc4c23)cc1 ; c1ccc(-c2ccc(Nc3ccccc3-c3cccc4ccccc34)c3oc4ccccc4c23)cc1]

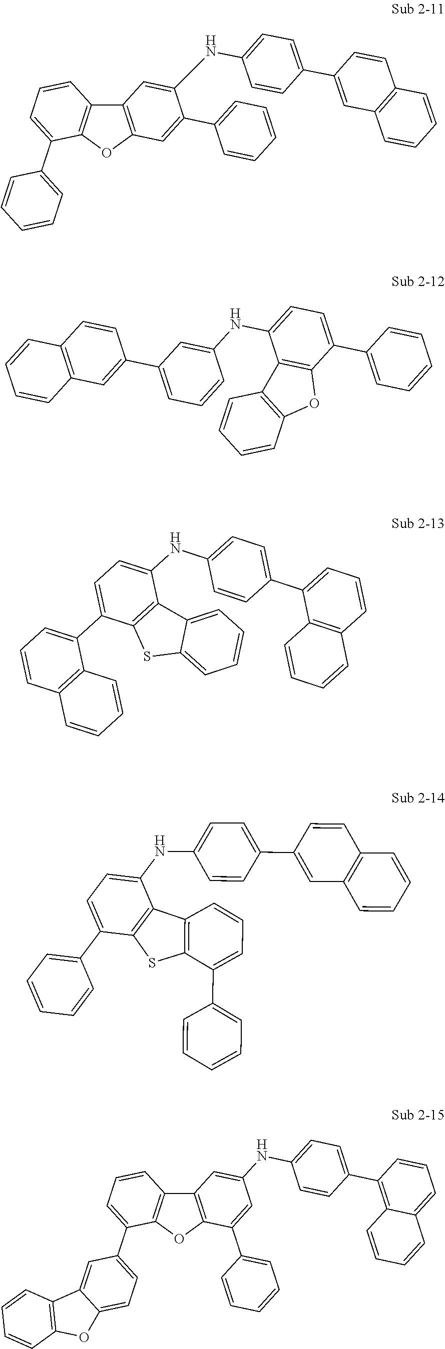 [SMILES: c1ccc(-c2cc3oc4c(-c5ccccc5)cccc4c3cc2Nc2ccc(-c3ccc4ccccc4c3)cc2)cc1 ; c1ccc(-c2ccc(Nc3cccc(-c4ccc5ccccc5c4)c3)c3c2oc2ccccc23)cc1 ; c1ccc2c(-c3ccc(Nc4ccc(-c5cccc6ccccc56)c5sc6ccccc6c45)cc3)cccc2c1 ; c1ccc(-c2cccc3c2sc2c(-c4ccccc4)ccc(Nc4ccc(-c5ccc6ccccc6c5)cc4)c23)cc1 ; c1ccc(-c2cc(Nc3ccc(-c4cccc5ccccc45)cc3)cc3c2oc2c(-c4ccc5oc6ccccc6c5c4)cccc23)cc1]

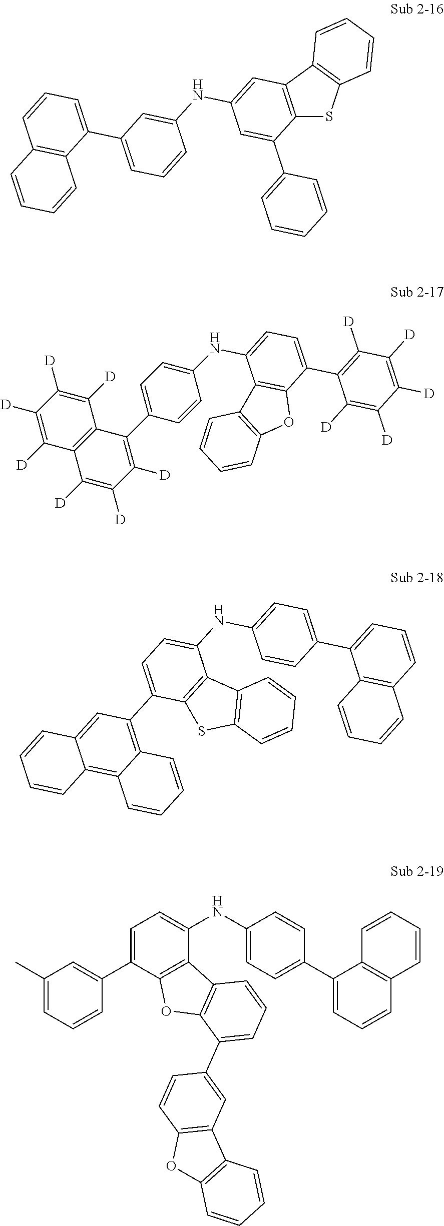 [SMILES: c1ccc(-c2cc(Nc3cccc(-c4cccc5ccccc45)c3)cc3c2sc2ccccc23)cc1 ; [2H]c1c([2H])c([2H])c(-c2ccc(Nc3ccc(-c4c([2H])c([2H])c([2H])c5c([2H])c([2H])c([2H])c([2H])c45)cc3)c3c2oc2ccccc23)c([2H])c1[2H] ; c1ccc2c(-c3ccc(Nc4ccc(-c5cc6ccccc6c6ccccc56)c5sc6ccccc6c45)cc3)cccc2c1 ; Cc1cccc(-c2ccc(Nc3ccc(-c4cccc5ccccc45)cc3)c3c2oc2c(-c4ccc5oc6ccccc6c5c4)cccc23)c1]

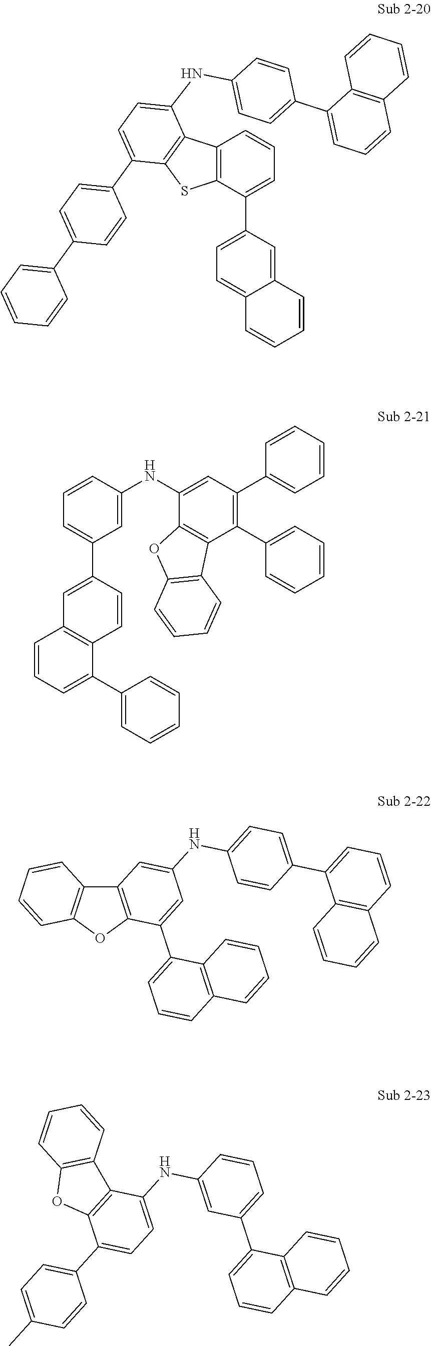 [SMILES: c1ccc(-c2ccc(-c3ccc(Nc4ccc(-c5cccc6ccccc56)cc4)c4c3sc3c(-c5ccc6ccccc6c5)cccc34)cc2)cc1 ; c1ccc(-c2cc(Nc3cccc(-c4ccc5c(-c6ccccc6)cccc5c4)c3)c3oc4ccccc4c3c2-c2ccccc2)cc1 ; c1ccc2c(-c3ccc(Nc4cc(-c5cccc6ccccc56)c5oc6ccccc6c5c4)cc3)cccc2c1 ; Cc1ccc(-c2ccc(Nc3cccc(-c4cccc5ccccc45)c3)c3c2oc2ccccc23)cc1]

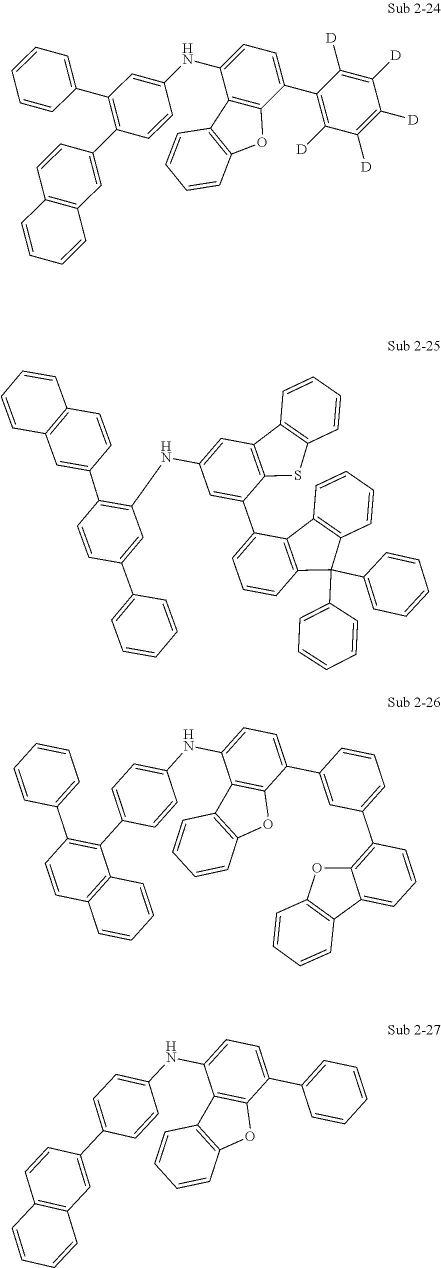 [SMILES: [2H]c1c([2H])c([2H])c(-c2ccc(Nc3ccc(-c4ccc5ccccc5c4)c(-c4ccccc4)c3)c3c2oc2ccccc23)c([2H])c1[2H] ; c1ccc(-c2ccc(-c3ccc4ccccc4c3)c(Nc3cc(-c4cccc5c4-c4ccccc4C5(c4ccccc4)c4ccccc4)c4sc5ccccc5c4c3)c2)cc1 ; c1ccc(-c2ccc3ccccc3c2-c2ccc(Nc3ccc(-c4cccc(-c5cccc6c5oc5ccccc56)c4)c4oc5ccccc5c34)cc2)cc1 ; c1ccc(-c2ccc(Nc3ccc(-c4ccc5ccccc5c4)cc3)c3c2oc2ccccc23)cc1]

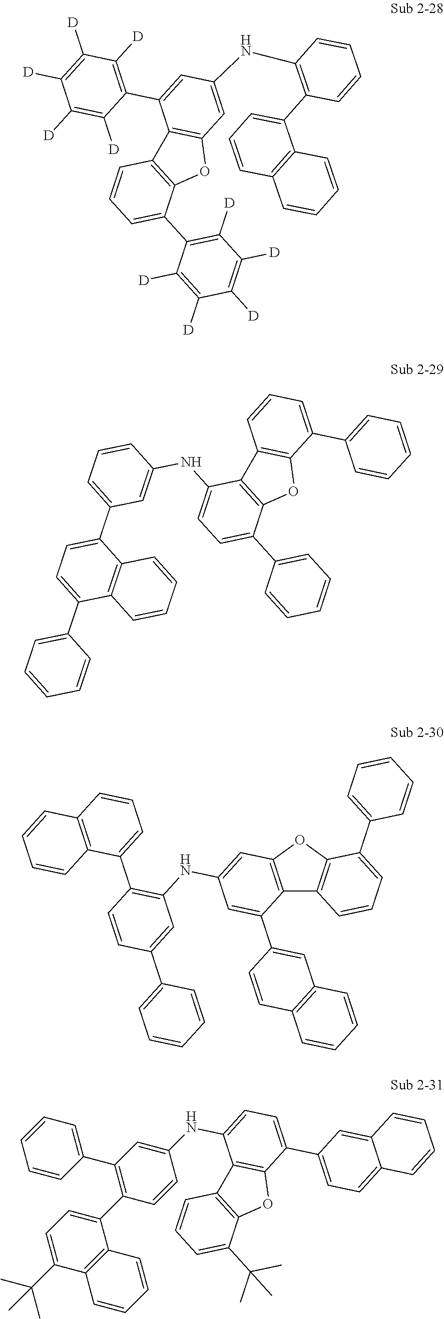 [SMILES: [2H]c1c([2H])c([2H])c(-c2cccc3c2oc2cc(Nc4ccccc4-c4cccc5ccccc45)cc(-c4c([2H])c([2H])c([2H])c([2H])c4[2H])c23)c([2H])c1[2H] ; c1ccc(-c2ccc(-c3cccc(Nc4ccc(-c5ccccc5)c5oc6c(-c7ccccc7)cccc6c45)c3)c3ccccc23)cc1 ; c1ccc(-c2ccc(-c3cccc4ccccc34)c(Nc3cc(-c4ccc5ccccc5c4)c4c(c3)oc3c(-c5ccccc5)cccc34)c2)cc1 ; CC(C)(C)c1ccc(-c2ccc(Nc3ccc(-c4ccc5ccccc5c4)c4oc5c(C(C)(C)C)cccc5c34)cc2-c2ccccc2)c2ccccc12]

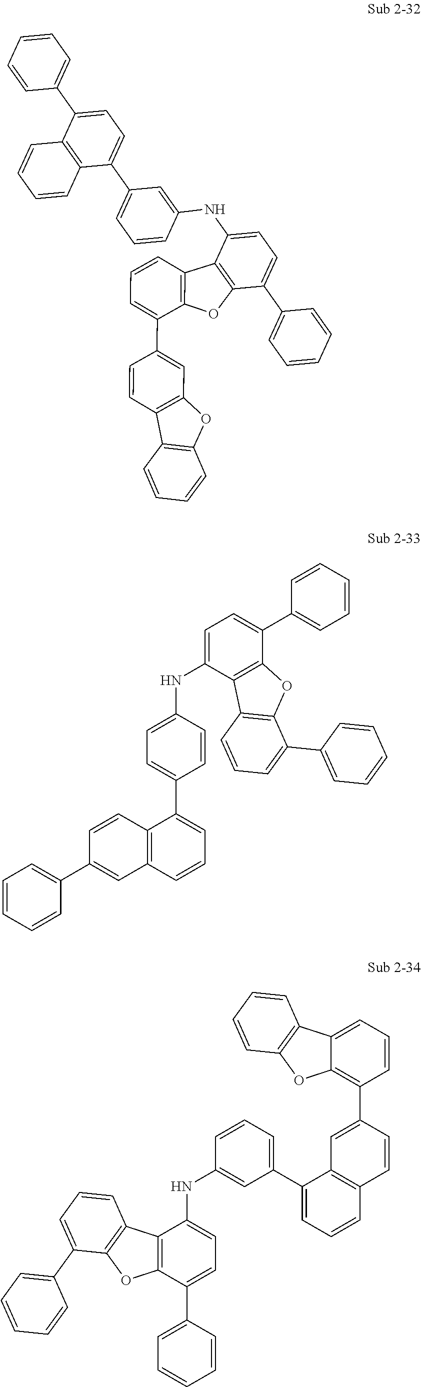 [SMILES: c1ccc(-c2ccc(-c3cccc(Nc4ccc(-c5ccccc5)c5oc6c(-c7ccc8c(c7)oc7ccccc78)cccc6c45)c3)c3ccccc23)cc1 ; c1ccc(-c2ccc3c(-c4ccc(Nc5ccc(-c6ccccc6)c6oc7c(-c8ccccc8)cccc7c56)cc4)cccc3c2)cc1 ; c1ccc(-c2cccc3c2oc2c(-c4ccccc4)ccc(Nc4cccc(-c5cccc6ccc(-c7cccc8c7oc7ccccc78)cc56)c4)c23)cc1]

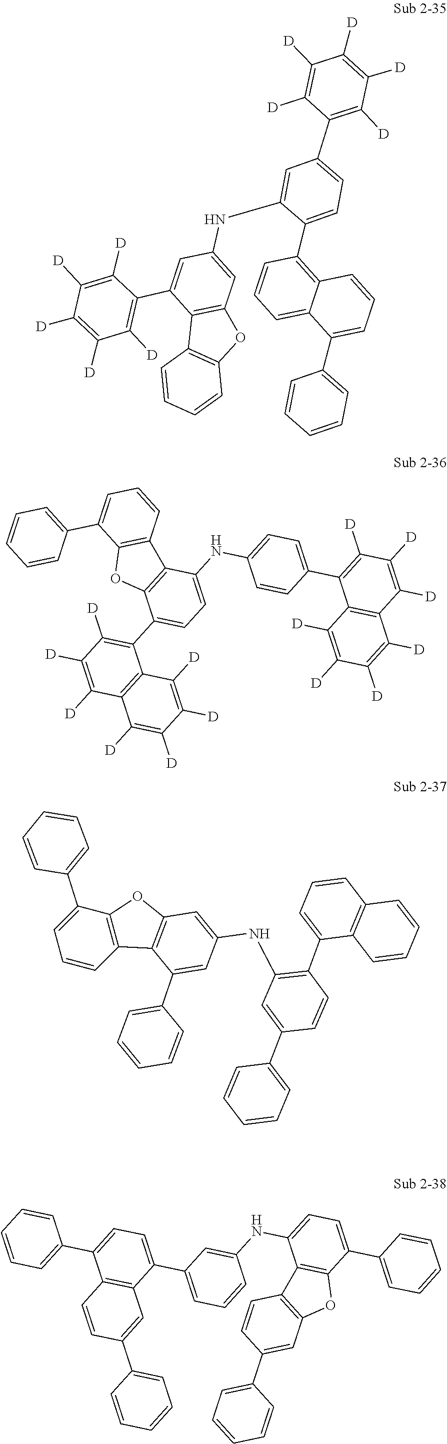 [SMILES: [2H]c1c([2H])c([2H])c(-c2ccc(-c3cccc4c(-c5ccccc5)cccc34)c(Nc3cc(-c4c([2H])c([2H])c([2H])c([2H])c4[2H])c4c(c3)oc3ccccc34)c2)c([2H])c1[2H] ; [2H]c1c([2H])c([2H])c2c(-c3ccc(Nc4ccc(-c5c([2H])c([2H])c([2H])c6c([2H])c([2H])c([2H])c([2H])c56)c5oc6c(-c7ccccc7)cccc6c45)cc3)c([2H])c([2H])c([2H])c2c1[2H] ; c1ccc(-c2ccc(-c3cccc4ccccc34)c(Nc3cc(-c4ccccc4)c4c(c3)oc3c(-c5ccccc5)cccc34)c2)cc1 ; c1ccc(-c2ccc3c(c2)oc2c(-c4ccccc4)ccc(Nc4cccc(-c5ccc(-c6ccccc6)c6ccc(-c7ccccc7)cc56)c4)c23)cc1]

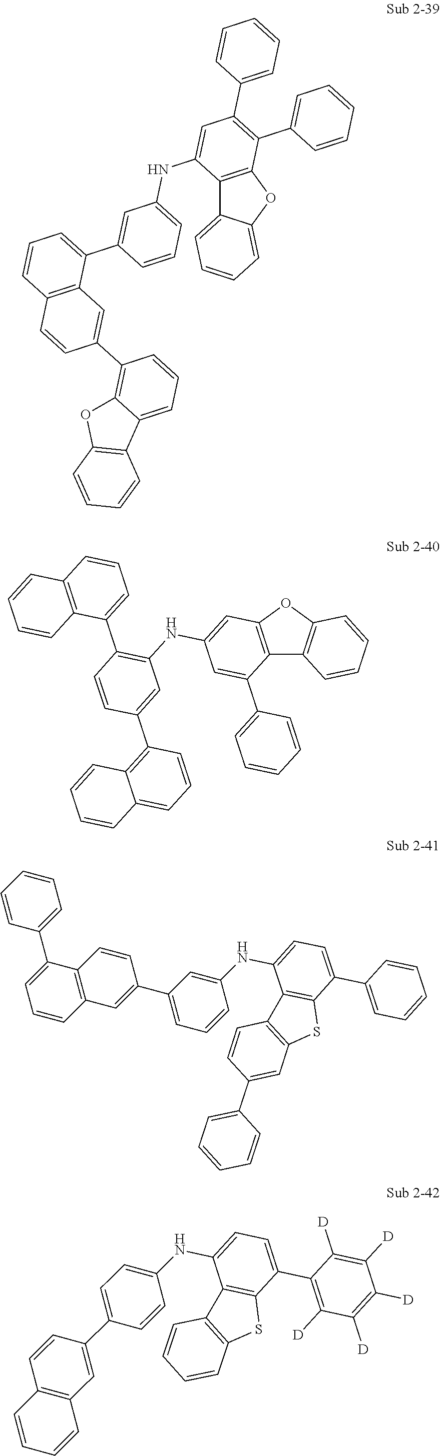 [SMILES: c1ccc(-c2cc(Nc3cccc(-c4cccc5ccc(-c6cccc7c6oc6ccccc67)cc45)c3)c3c(oc4ccccc43)c2-c2ccccc2)cc1 ; c1ccc(-c2cc(Nc3cc(-c4cccc5ccccc45)ccc3-c3cccc4ccccc34)cc3oc4ccccc4c23)cc1 ; c1ccc(-c2ccc3c(c2)sc2c(-c4ccccc4)ccc(Nc4cccc(-c5ccc6c(-c7ccccc7)cccc6c5)c4)c23)cc1 ; [2H]c1c([2H])c([2H])c(-c2ccc(Nc3ccc(-c4ccc5ccccc5c4)cc3)c3c2sc2ccccc23)c([2H])c1[2H]]

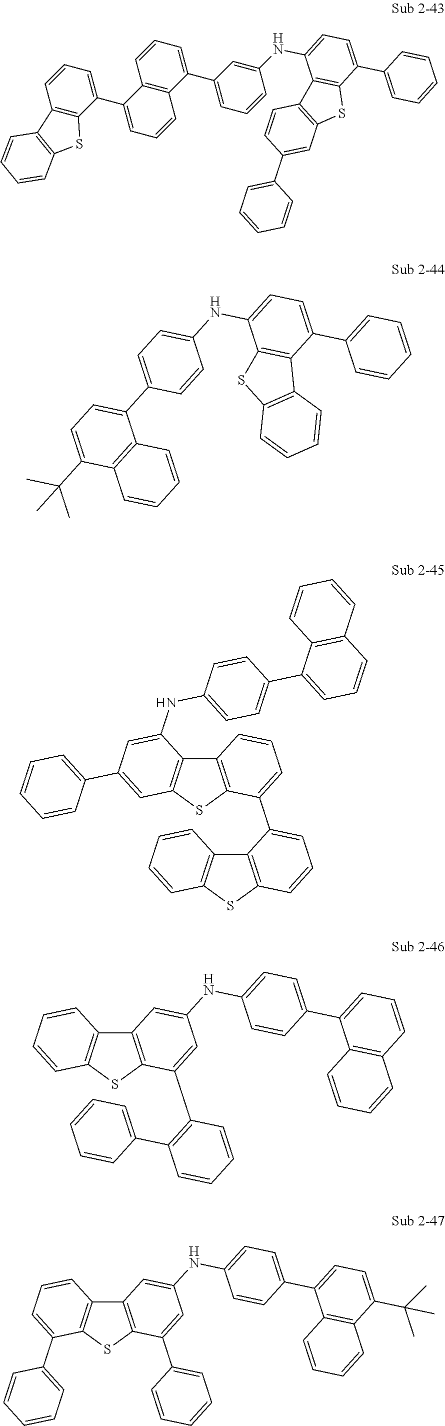 [SMILES: c1ccc(-c2ccc3c(c2)sc2c(-c4ccccc4)ccc(Nc4cccc(-c5cccc6c(-c7cccc8c7sc7ccccc78)cccc56)c4)c23)cc1 ; CC(C)(C)c1ccc(-c2ccc(Nc3ccc(-c4ccccc4)c4c3sc3ccccc34)cc2)c2ccccc12 ; c1ccc(-c2cc(Nc3ccc(-c4cccc5ccccc45)cc3)c3c(c2)sc2c(-c4cccc5sc6ccccc6c45)cccc23)cc1 ; c1ccc(-c2ccccc2-c2cc(Nc3ccc(-c4cccc5ccccc45)cc3)cc3c2sc2ccccc23)cc1 ; CC(C)(C)c1ccc(-c2ccc(Nc3cc(-c4ccccc4)c4sc5c(-c6ccccc6)cccc5c4c3)cc2)c2ccccc12]

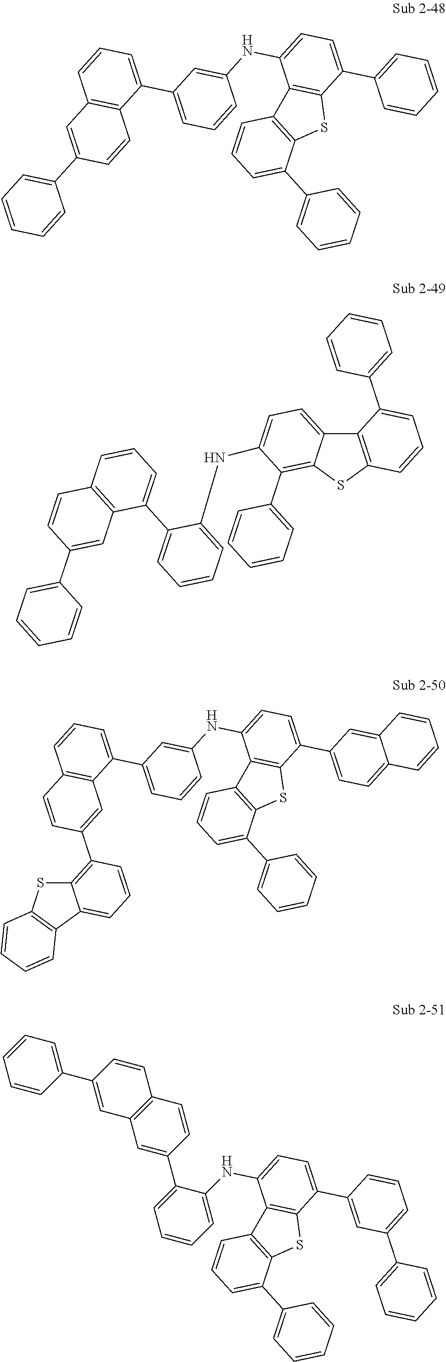 [SMILES: c1ccc(-c2ccc3c(-c4cccc(Nc5ccc(-c6ccccc6)c6sc7c(-c8ccccc8)cccc7c56)c4)cccc3c2)cc1 ; c1ccc(-c2ccc3cccc(-c4ccccc4Nc4ccc5c(sc6cccc(-c7ccccc7)c65)c4-c4ccccc4)c3c2)cc1 ; c1ccc(-c2cccc3c2sc2c(-c4ccc5ccccc5c4)ccc(Nc4cccc(-c5cccc6ccc(-c7cccc8c7sc7ccccc78)cc56)c4)c23)cc1 ; c1ccc(-c2cccc(-c3ccc(Nc4ccccc4-c4ccc5ccc(-c6ccccc6)cc5c4)c4c3sc3c(-c5ccccc5)cccc34)c2)cc1]

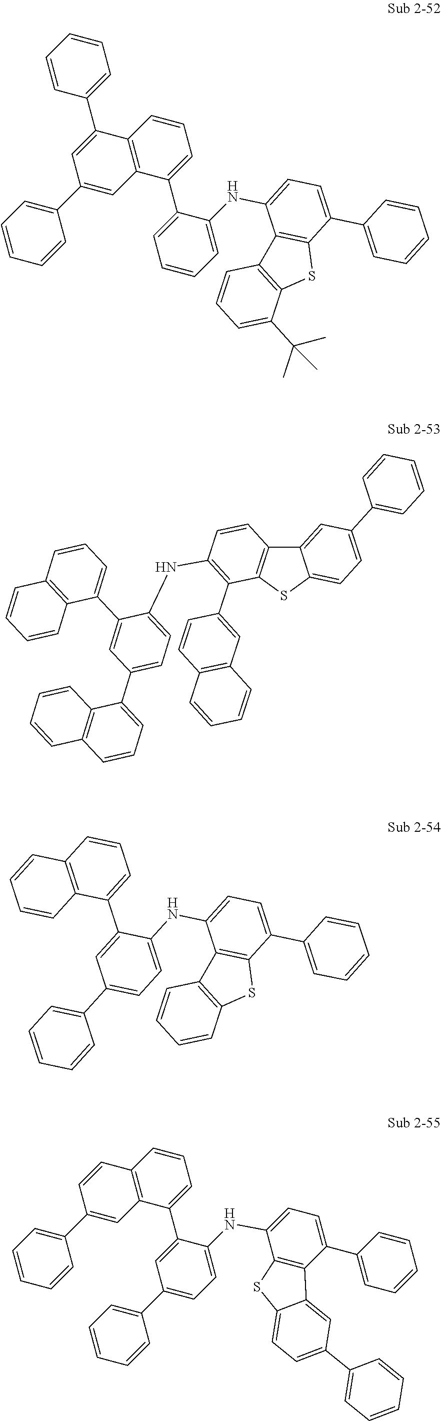 [SMILES: CC(C)(C)c1cccc2c1sc1c(-c3ccccc3)ccc(Nc3ccccc3-c3cccc4c(-c5ccccc5)cc(-c5ccccc5)cc34)c12 ; c1ccc(-c2ccc3sc4c(-c5ccc6ccccc6c5)c(Nc5ccc(-c6cccc7ccccc67)cc5-c5cccc6ccccc56)ccc4c3c2)cc1 ; c1ccc(-c2ccc(Nc3ccc(-c4ccccc4)c4sc5ccccc5c34)c(-c3cccc4ccccc34)c2)cc1 ; c1ccc(-c2ccc(Nc3ccc(-c4ccccc4)c4c3sc3ccc(-c5ccccc5)cc34)c(-c3cccc4ccc(-c5ccccc5)cc34)c2)cc1]

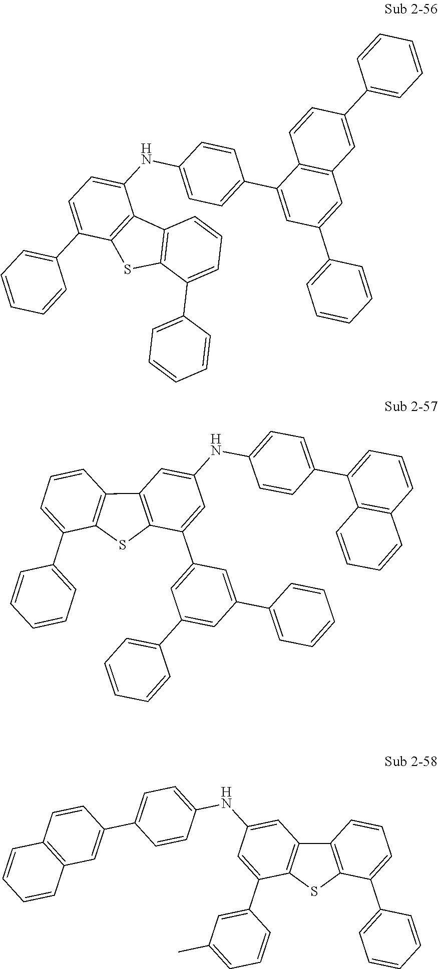 [SMILES: c1ccc(-c2ccc3c(-c4ccc(Nc5ccc(-c6ccccc6)c6sc7c(-c8ccccc8)cccc7c56)cc4)cc(-c4ccccc4)cc3c2)cc1 ; c1ccc(-c2cc(-c3ccccc3)cc(-c3cc(Nc4ccc(-c5cccc6ccccc56)cc4)cc4c3sc3c(-c5ccccc5)cccc34)c2)cc1 ; Cc1cccc(-c2cc(Nc3ccc(-c4ccc5ccccc5c4)cc3)cc3c2sc2c(-c4ccccc4)cccc23)c1]

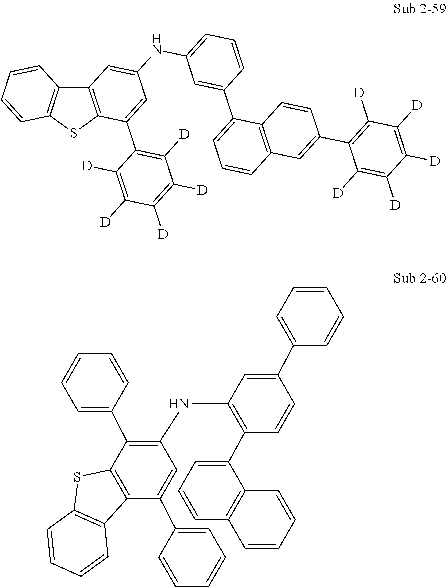 [SMILES: [2H]c1c([2H])c([2H])c(-c2ccc3c(-c4cccc(Nc5cc(-c6c([2H])c([2H])c([2H])c([2H])c6[2H])c6sc7ccccc7c6c5)c4)cccc3c2)c([2H])c1[2H] ; c1ccc(-c2ccc(-c3cccc4ccccc34)c(Nc3cc(-c4ccccc4)c4c(sc5ccccc54)c3-c3ccccc3)c2)cc1]

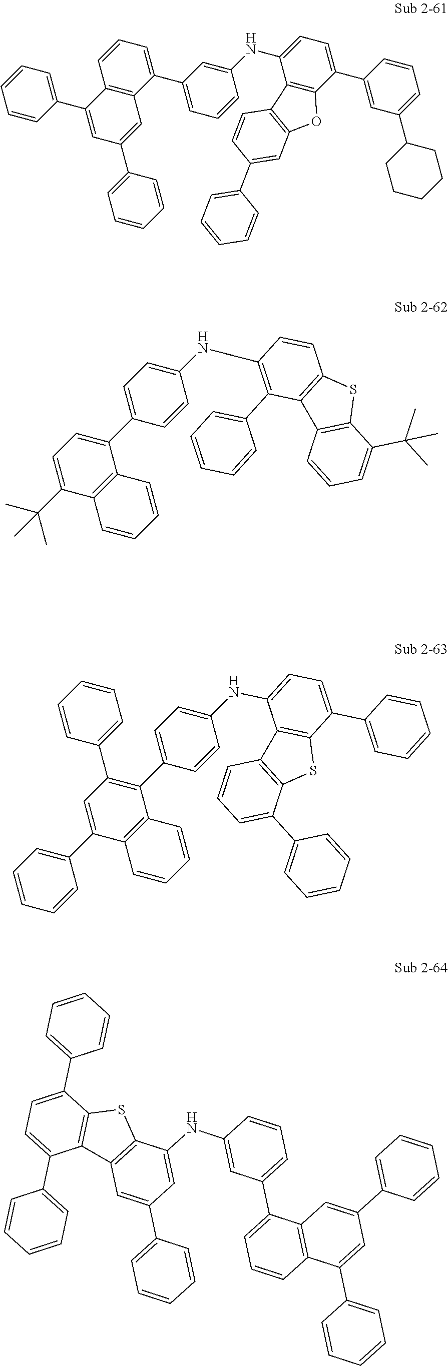 [SMILES: c1ccc(-c2ccc3c(c2)oc2c(-c4cccc(C5CCCCC5)c4)ccc(Nc4cccc(-c5cccc6c(-c7ccccc7)cc(-c7ccccc7)cc56)c4)c23)cc1 ; CC(C)(C)c1ccc(-c2ccc(Nc3ccc4sc5c(C(C)(C)C)cccc5c4c3-c3ccccc3)cc2)c2ccccc12 ; c1ccc(-c2cc(-c3ccccc3)c3ccccc3c2-c2ccc(Nc3ccc(-c4ccccc4)c4sc5c(-c6ccccc6)cccc5c34)cc2)cc1 ; c1ccc(-c2cc(-c3ccccc3)c3cccc(-c4cccc(Nc5cc(-c6ccccc6)cc6c5sc5c(-c7ccccc7)ccc(-c7ccccc7)c56)c4)c3c2)cc1]

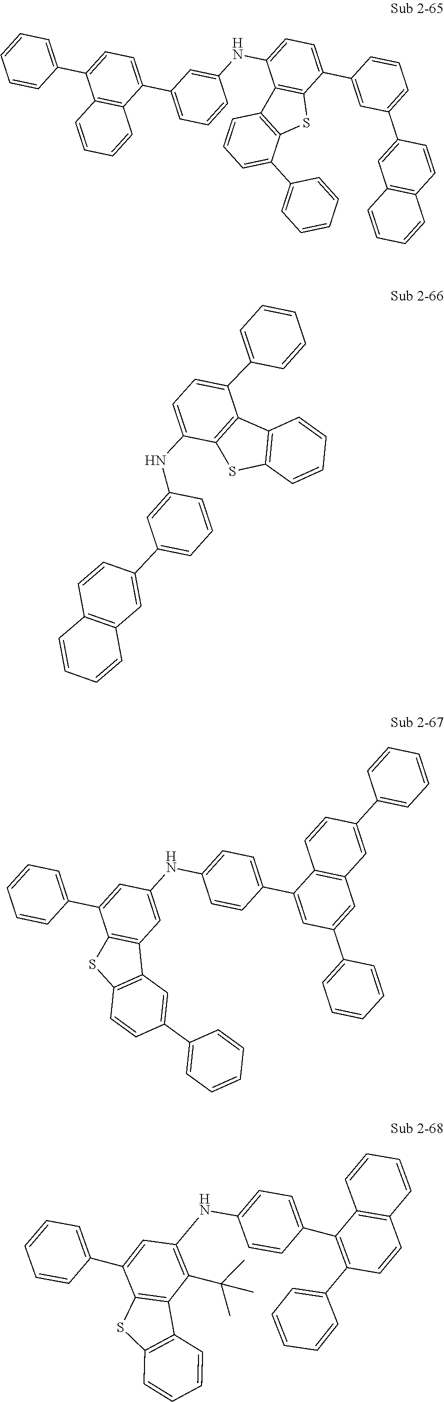 [SMILES: c1ccc(-c2ccc(-c3cccc(Nc4ccc(-c5cccc(-c6ccc7ccccc7c6)c5)c5sc6c(-c7ccccc7)cccc6c45)c3)c3ccccc23)cc1 ; c1ccc(-c2ccc(Nc3cccc(-c4ccc5ccccc5c4)c3)c3sc4ccccc4c23)cc1 ; c1ccc(-c2ccc3c(-c4ccc(Nc5cc(-c6ccccc6)c6sc7ccc(-c8ccccc8)cc7c6c5)cc4)cc(-c4ccccc4)cc3c2)cc1 ; CC(C)(C)c1c(Nc2ccc(-c3c(-c4ccccc4)ccc4ccccc34)cc2)cc(-c2ccccc2)c2sc3ccccc3c12]

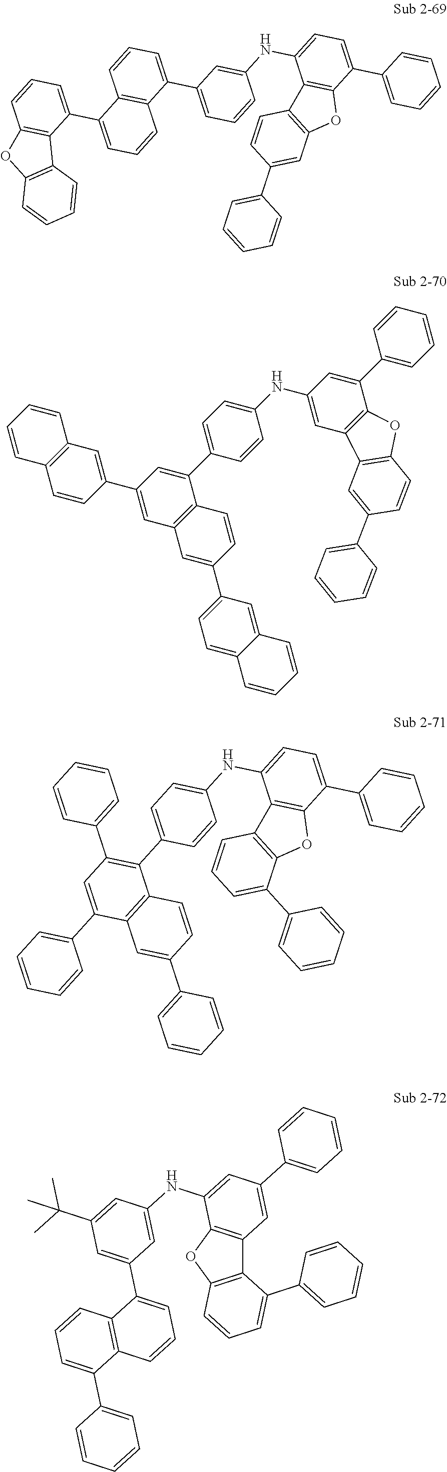 [SMILES: c1ccc(-c2ccc3c(c2)oc2c(-c4ccccc4)ccc(Nc4cccc(-c5cccc6c(-c7cccc8oc9ccccc9c78)cccc56)c4)c23)cc1 ; c1ccc(-c2ccc3oc4c(-c5ccccc5)cc(Nc5ccc(-c6cc(-c7ccc8ccccc8c7)cc7cc(-c8ccc9ccccc9c8)ccc67)cc5)cc4c3c2)cc1 ; c1ccc(-c2ccc3c(-c4ccc(Nc5ccc(-c6ccccc6)c6oc7c(-c8ccccc8)cccc7c56)cc4)c(-c4ccccc4)cc(-c4ccccc4)c3c2)cc1 ; CC(C)(C)c1cc(Nc2cc(-c3ccccc3)cc3c2oc2cccc(-c4ccccc4)c23)cc(-c2cccc3c(-c4ccccc4)cccc23)c1]

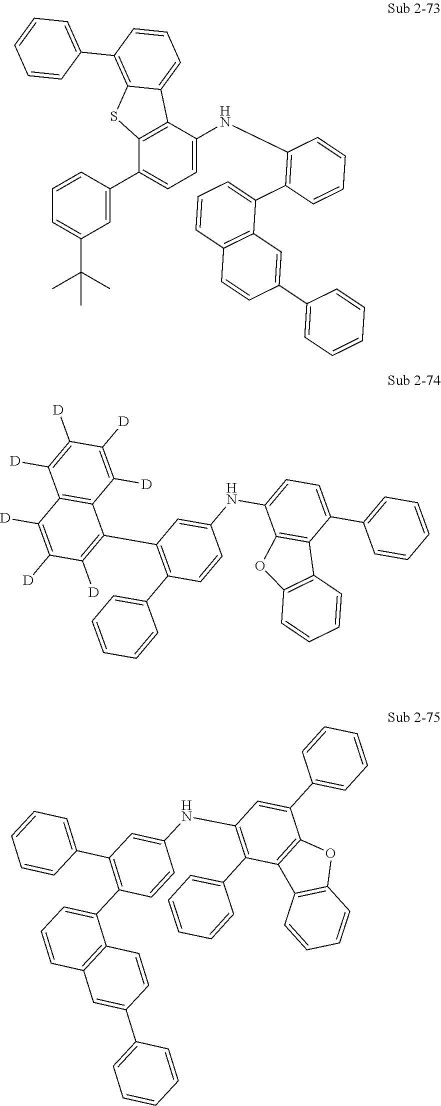 [SMILES: CC(C)(C)c1cccc(-c2ccc(Nc3ccccc3-c3cccc4ccc(-c5ccccc5)cc34)c3c2sc2c(-c4ccccc4)cccc23)c1 ; [2H]c1c([2H])c([2H])c2c(-c3cc(Nc4ccc(-c5ccccc5)c5c4oc4ccccc45)ccc3-c3ccccc3)c([2H])c([2H])c([2H])c2c1[2H] ; c1ccc(-c2ccc3c(-c4ccc(Nc5cc(-c6ccccc6)c6oc7ccccc7c6c5-c5ccccc5)cc4-c4ccccc4)cccc3c2)cc1]

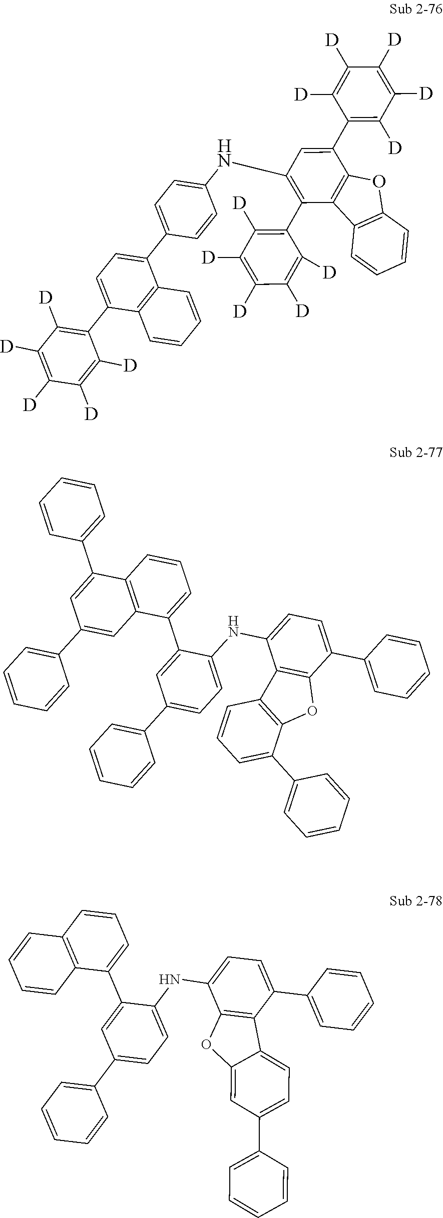 [SMILES: [2H]c1c([2H])c([2H])c(-c2ccc(-c3ccc(Nc4cc(-c5c([2H])c([2H])c([2H])c([2H])c5[2H])c5oc6ccccc6c5c4-c4c([2H])c([2H])c([2H])c([2H])c4[2H])cc3)c3ccccc23)c([2H])c1[2H] ; c1ccc(-c2ccc(Nc3ccc(-c4ccccc4)c4oc5c(-c6ccccc6)cccc5c34)c(-c3cccc4c(-c5ccccc5)cc(-c5ccccc5)cc34)c2)cc1 ; c1ccc(-c2ccc(Nc3ccc(-c4ccccc4)c4c3oc3cc(-c5ccccc5)ccc34)c(-c3cccc4ccccc34)c2)cc1]

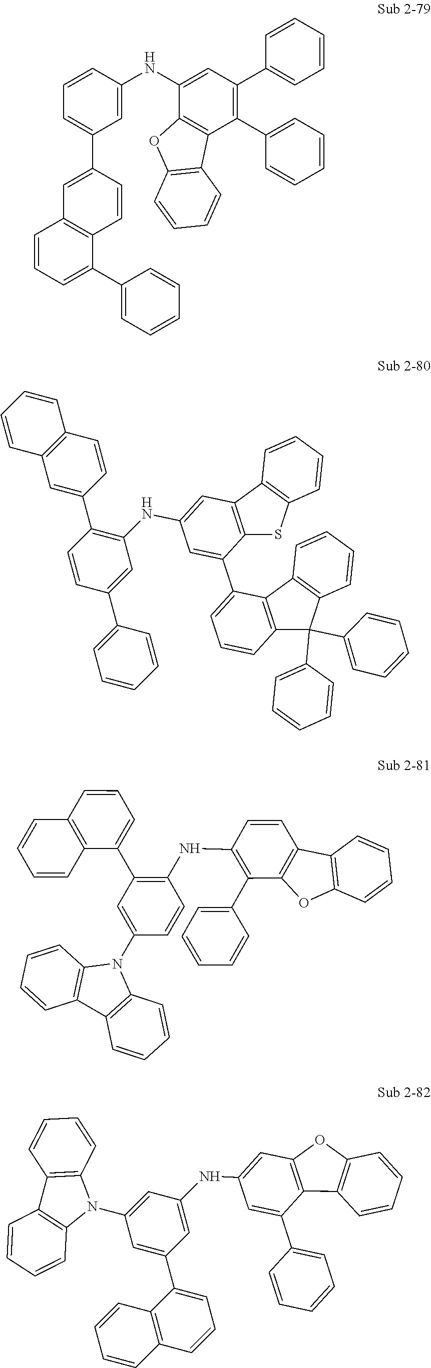 [SMILES: c1ccc(-c2cc(Nc3cccc(-c4ccc5c(-c6ccccc6)cccc5c4)c3)c3oc4ccccc4c3c2-c2ccccc2)cc1 ; c1ccc(-c2ccc(-c3ccc4ccccc4c3)c(Nc3cc(-c4cccc5c4-c4ccccc4C5(c4ccccc4)c4ccccc4)c4sc5ccccc5c4c3)c2)cc1 ; c1ccc(-c2c(Nc3ccc(-n4c5ccccc5c5ccccc54)cc3-c3cccc4ccccc34)ccc3c2oc2ccccc23)cc1 ; c1ccc(-c2cc(Nc3cc(-c4cccc5ccccc45)cc(-n4c5ccccc5c5ccccc54)c3)cc3oc4ccccc4c23)cc1]

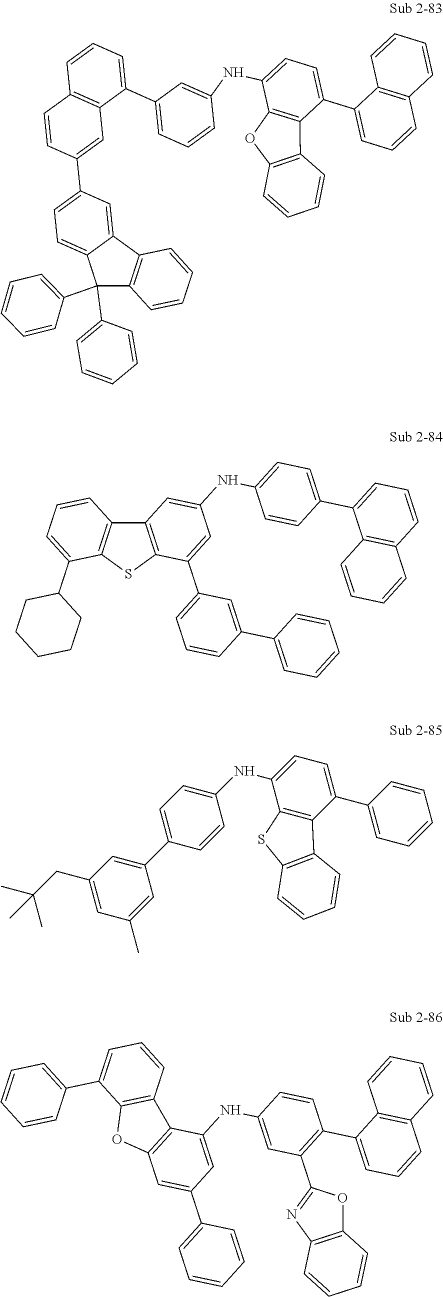 [SMILES: c1ccc(C2(c3ccccc3)c3ccccc3-c3cc(-c4ccc5cccc(-c6cccc(Nc7ccc(-c8cccc9ccccc89)c8c7oc7ccccc78)c6)c5c4)ccc32)cc1 ; c1ccc(-c2cccc(-c3cc(Nc4ccc(-c5cccc6ccccc56)cc4)cc4c3sc3c(C5CCCCC5)cccc34)c2)cc1 ; Cc1cc(CC(C)(C)C)cc(-c2ccc(Nc3ccc(-c4ccccc4)c4c3sc3ccccc34)cc2)c1 ; c1ccc(-c2cc(Nc3ccc(-c4cccc5ccccc45)c(-c4nc5ccccc5o4)c3)c3c(c2)oc2c(-c4ccccc4)cccc23)cc1]

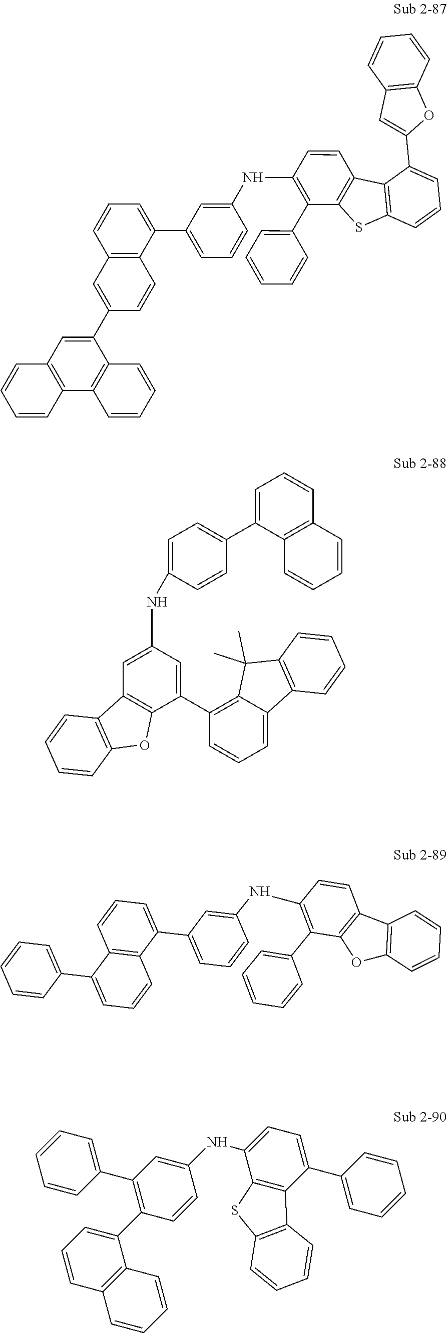 [SMILES: c1ccc(-c2c(Nc3cccc(-c4cccc5cc(-c6cc7ccccc7c7ccccc67)ccc45)c3)ccc3c2sc2cccc(-c4cc5ccccc5o4)c23)cc1 ; CC1(C)c2ccccc2-c2cccc(-c3cc(Nc4ccc(-c5cccc6ccccc56)cc4)cc4c3oc3ccccc34)c21 ; c1ccc(-c2cccc3c(-c4cccc(Nc5ccc6c(oc7ccccc76)c5-c5ccccc5)c4)cccc23)cc1 ; c1ccc(-c2cc(Nc3ccc(-c4ccccc4)c4c3sc3ccccc34)ccc2-c2cccc3ccccc23)cc1]

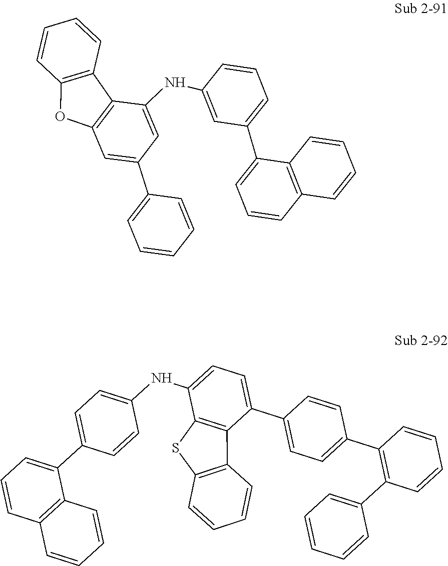 [SMILES: c1ccc(-c2cc(Nc3cccc(-c4cccc5ccccc45)c3)c3c(c2)oc2ccccc23)cc1 ; c1ccc(-c2ccccc2-c2ccc(-c3ccc(Nc4ccc(-c5cccc6ccccc56)cc4)c4sc5ccccc5c34)cc2)cc1]

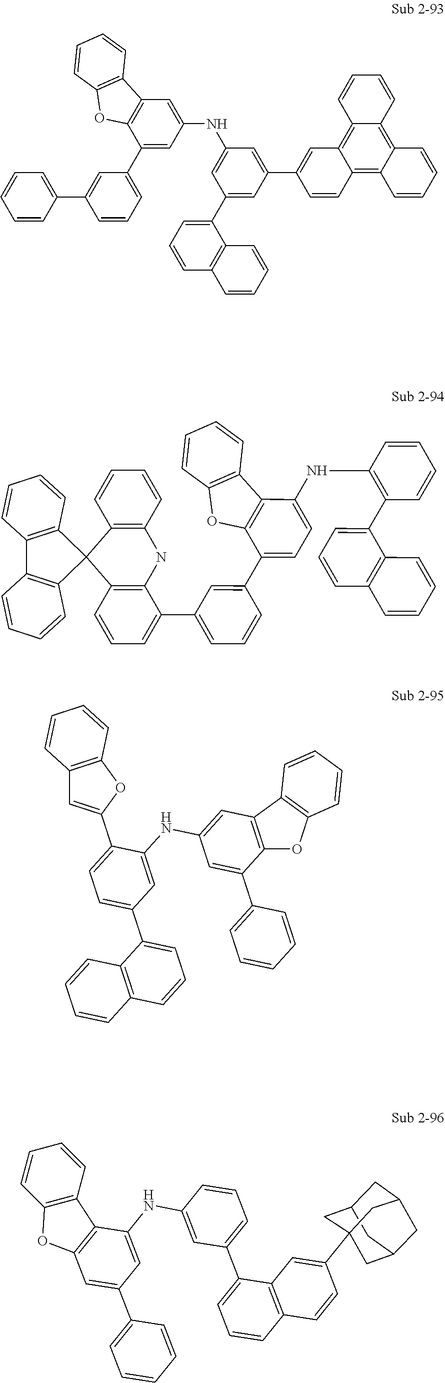 [SMILES: c1ccc(-c2cccc(-c3cc(Nc4cc(-c5ccc6c7ccccc7c7ccccc7c6c5)cc(-c5cccc6ccccc56)c4)cc4c3oc3ccccc34)c2)cc1 ; c1cc(-c2cccc3c2Nc2ccccc2C32c3ccccc3-c3ccccc32)cc(-c2ccc(Nc3ccccc3-c3cccc4ccccc34)c3c2oc2ccccc23)c1 ; c1ccc(-c2cc(Nc3cc(-c4cccc5ccccc45)ccc3-c3cc4ccccc4o3)cc3c2oc2ccccc23)cc1 ; c1ccc(-c2cc(Nc3cccc(-c4cccc5ccc(C67CC8CC(CC(C8)C6)C7)cc45)c3)c3c(c2)oc2ccccc23)cc1]

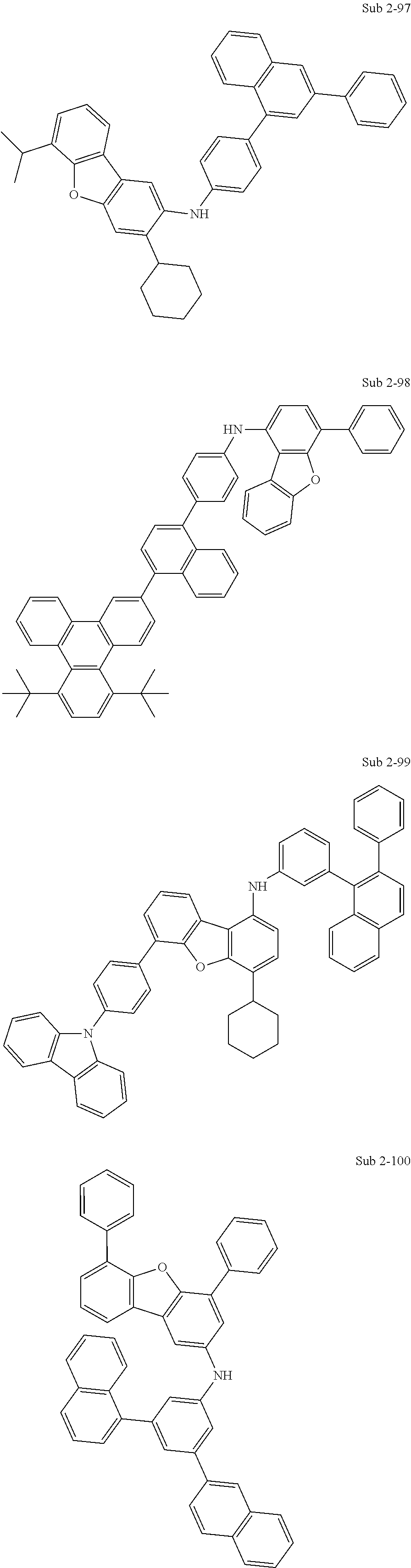 [SMILES: CC(C)c1cccc2c1oc1cc(C3CCCCC3)c(Nc3ccc(-c4cc(-c5ccccc5)cc5ccccc45)cc3)cc12 ; CC(C)(C)c1ccc(C(C)(C)C)c2c3ccc(-c4ccc(-c5ccc(Nc6ccc(-c7ccccc7)c7oc8ccccc8c67)cc5)c5ccccc45)cc3c3ccccc3c12 ; c1ccc(-c2ccc3ccccc3c2-c2cccc(Nc3ccc(C4CCCCC4)c4oc5c(-c6ccc(-n7c8ccccc8c8ccccc87)cc6)cccc5c34)c2)cc1 ; c1ccc(-c2cccc3c2oc2c(-c4ccccc4)cc(Nc4cc(-c5ccc6ccccc6c5)cc(-c5cccc6ccccc56)c4)cc23)cc1]

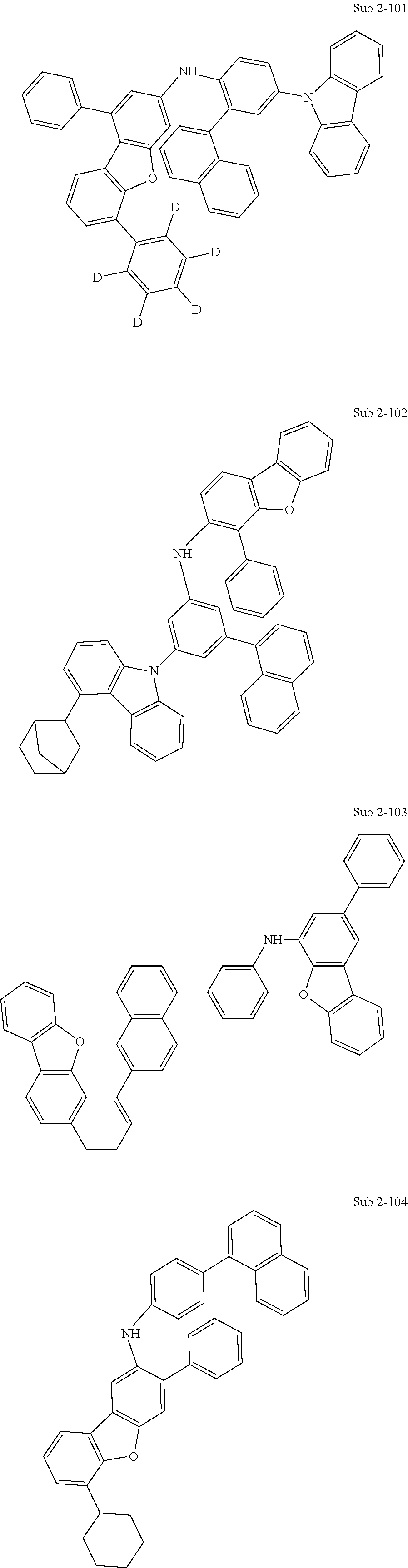 [SMILES: [2H]c1c([2H])c([2H])c(-c2cccc3c2oc2cc(Nc4ccc(-n5c6ccccc6c6ccccc65)cc4-c4cccc5ccccc45)cc(-c4ccccc4)c23)c([2H])c1[2H] ; c1ccc(-c2c(Nc3cc(-c4cccc5ccccc45)cc(-n4c5ccccc5c5c(C6CC7CCC6C7)cccc54)c3)ccc3c2oc2ccccc23)cc1 ; c1ccc(-c2cc(Nc3cccc(-c4cccc5cc(-c6cccc7ccc8c9ccccc9oc8c67)ccc45)c3)c3oc4ccccc4c3c2)cc1 ; c1ccc(-c2cc3oc4c(C5CCCCC5)cccc4c3cc2Nc2ccc(-c3cccc4ccccc34)cc2)cc1]

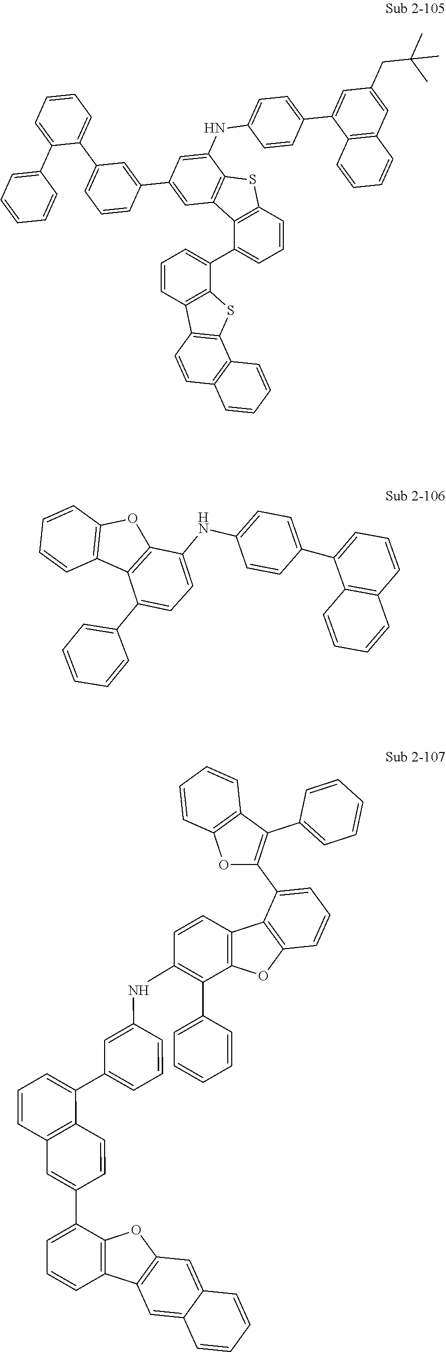 [SMILES: CC(C)(C)Cc1cc(-c2ccc(Nc3cc(-c4cccc(-c5ccccc5-c5ccccc5)c4)cc4c3sc3cccc(-c5cccc6c5sc5c7ccccc7ccc65)c34)cc2)c2ccccc2c1 ; c1ccc(-c2ccc(Nc3ccc(-c4cccc5ccccc45)cc3)c3oc4ccccc4c23)cc1 ; c1ccc(-c2c(-c3cccc4oc5c(-c6ccccc6)c(Nc6cccc(-c7cccc8cc(-c9cccc%10c9oc9cc%11ccccc%11cc9%10)ccc78)c6)ccc5c34)oc3ccccc23)cc1]

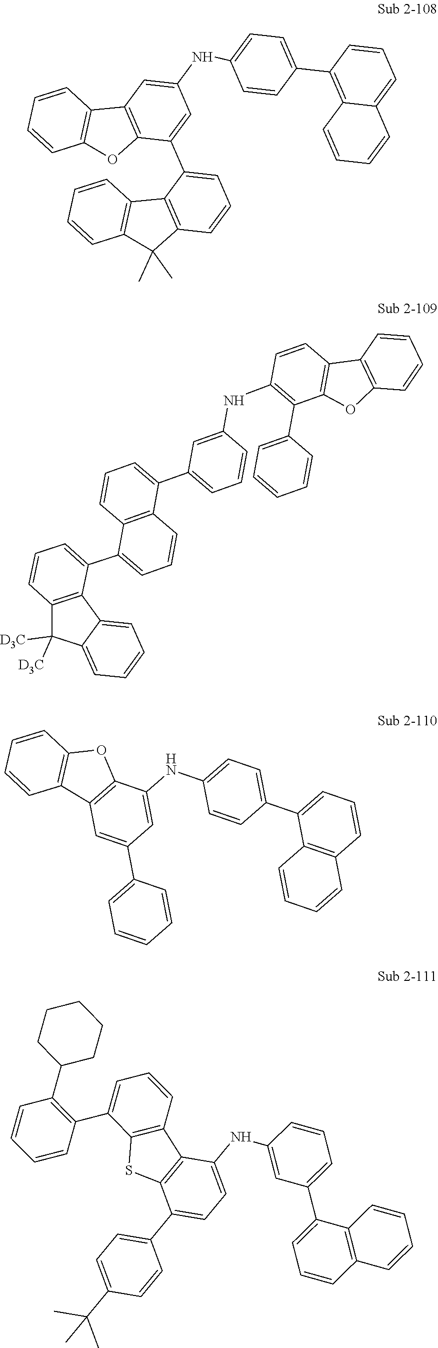 [SMILES: CC1(C)c2ccccc2-c2c(-c3cc(Nc4ccc(-c5cccc6ccccc56)cc4)cc4c3oc3ccccc34)cccc21 ; [2H]C([2H])([2H])C1(C([2H])([2H])[2H])c2ccccc2-c2c(-c3cccc4c(-c5cccc(Nc6ccc7c(oc8ccccc87)c6-c6ccccc6)c5)cccc34)cccc21 ; c1ccc(-c2cc(Nc3ccc(-c4cccc5ccccc45)cc3)c3oc4ccccc4c3c2)cc1 ; CC(C)(C)c1ccc(-c2ccc(Nc3cccc(-c4cccc5ccccc45)c3)c3c2sc2c(-c4ccccc4C4CCCCC4)cccc23)cc1]

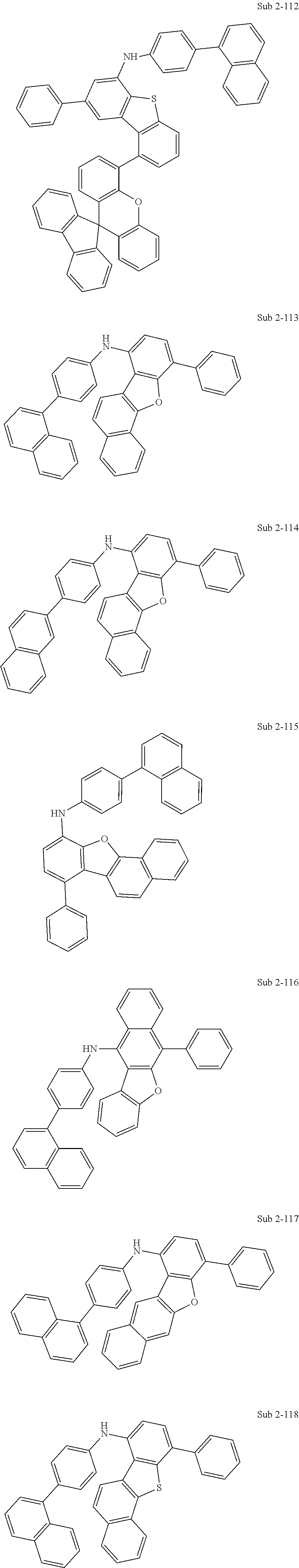

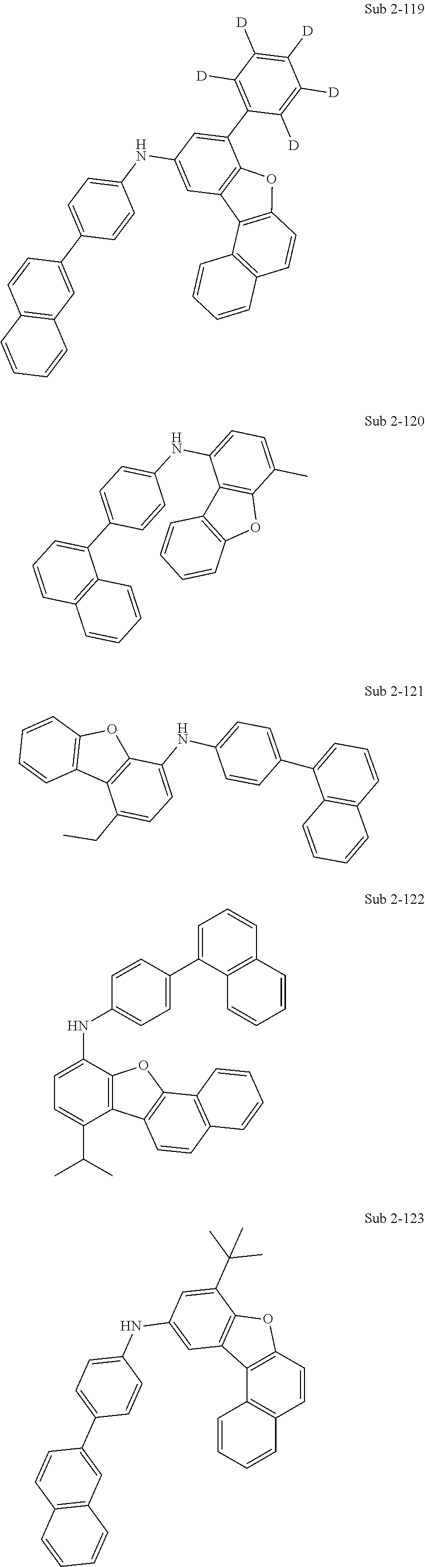 [SMILES: [2H]c1c([2H])c([2H])c(-c2cc(Nc3ccc(-c4ccc5ccccc5c4)cc3)cc3c2oc2ccc4ccccc4c23)c([2H])c1[2H] ; Cc1ccc(Nc2ccc(-c3cccc4ccccc34)cc2)c2c1oc1ccccc12 ; CCc1ccc(Nc2ccc(-c3cccc4ccccc34)cc2)c2oc3ccccc3c12 ; CC(C)c1ccc(Nc2ccc(-c3cccc4ccccc34)cc2)c2oc3c4ccccc4ccc3c12 ; CC(C)(C)c1cc(Nc2ccc(-c3ccc4ccccc4c3)cc2)cc2c1oc1ccc3ccccc3c12]

TABLE 2

| Compound | FD-MS | Compound | FD-MS |
|---|---|---|---|
| Sub 2-1 | m/z = 537.21(C₄₀H₂₇NO = 537.66) | Sub 2-2 | m/z = 461.18(C₃₄H₂₃NO = 461.56) |
| Sub 2-3 | m/z = 461.18(C₃₄H₂₃NO = 461.56) | Sub 2-4 | m/z = 461.18(C₃₄H₂₃NO = 461.56) |
| Sub 2-5 | m/z = 537.21(C₄₀H₂₇NO = 537.66) | Sub 2-6 | m/z = 537.21(C₄₀H₂₇NO = 537.66) |
| Sub 2-7 | m/z = 537.21(C₄₀H₂₇NO = 537.66) | Sub 2-8 | m/z = 461.18(C₃₄H₂₃NO = 461.56) |
| Sub 2-9 | m/z = 461.18(C₃₄H₂₃NO = 461.56) | Sub 2-10 | m/z = 461.18(C₃₄H₂₃NO = 461.56) |
| Sub 2-11 | m/z = 537.21(C₄₀H₂₇NO = 537.66) | Sub 2-12 | m/z = 461.18(C₃₄H₂₃NO = 461.56) |
| Sub 2-13 | m/z = 527.17(C₃₈H₂₅NS = 527.68) | Sub 2-14 | m/z = 553.19(C₄₀H₂₇NS = 553.72) |
| Sub 2-15 | m/z = 627.22(C₄₆H₂₉NO₂ = 627.74) | Sub 2-16 | m/z = 477.16(C₃₄H₂₃NS = 477.63) |
| Sub 2-17 | m/z = 473.25(C₃₄H₁₁D₁₂NO = 473.64) | Sub 2-18 | m/z = 577.19(C₄₂H₂₇NS = 577.74) |
| Sub 2-19 | m/z = 641.24(C₄₇H₃₁NO₂ = 641.77) | Sub 2-20 | m/z = 679.23(C₅₀H₃₃NS = 679.88) |
| Sub 2-21 | m/z = 613.24(C₄₆H₃₁NO = 613.76) | Sub 2-22 | m/z = 511.19(C₃₈H₂₅NO = 511.62) |
| Sub 2-23 | m/z = 475.19(C₃₅H₂₅NO = 475.59) | Sub 2-24 | m/z = 542.24(C₄₀H₂₂D₅NO = 542.69) |
| Sub 2-25 | m/z = 793.28(C₅₉H₃₉NS = 794.03) | Sub 2-26 | m/z = 703.25(C₅₂H₃₃NO₂ = 703.84) |
| Sub 2-27 | m/z = 461.18(C₃₄H₂₃NO = 461.56) | Sub 2-28 | m/z = 547.27(C₄₀H₁₇D₁₀NO = 547.72) |
| Sub 2-29 | m/z = 613.24(C₄₆H₃₁NO = 613.76) | Sub 2-30 | m/z = 663.26(C₅₀H₃₃NO = 663.82) |
| Sub 2-31 | m/z = 699.35(C₅₂H₄₅NO = 699.94) | Sub 2-32 | m/z = 703.25(C₅₂H₃₃NO₂ = 703.84) |
| Sub 2-33 | m/z = 613.24(C₄₆H₃₁NO = 613.76) | Sub 2-34 | m/z = 703.25(C₅₂H₃₃NO₂ = 703.84) |
| Sub 2-35 | m/z = 623.3(C₄₆H₂₁D₁₀NO = 623.82) | Sub 2-36 | m/z = 601.31(C₄₄H₁₅D₁₄NO = 601.81) |
| Sub 2-37 | m/z = 613.24(C₄₆H₃₁NO = 613.76) | Sub 2-38 | m/z = 689.27(C₅₂H₃₅NO = 689.86) |
| Sub 2-39 | m/z = 703.25(C₅₂H₃₃NO₂ = 703.84) | Sub 2-40 | m/z = 587.22(C₄₄H₂₉NO = 587.72) |
| Sub 2-41 | m/z = 629.22(C₄₆H₃₁NS = 629.82) | Sub 2-42 | m/z = 482.19(C₃₄H₁₈D₅NS = 482.66) |
| Sub 2-43 | m/z = 735.21(C₅₂H₃₃NS₂ = 735.96) | Sub 2-44 | m/z = 533.22(C₃₈H₃₁NS = 533.73) |
| Sub 2-45 | m/z = 659.17(C₄₆H₂₉NS₂ = 659.86) | Sub 2-46 | m/z = 553.19(C₄₀H₂₇NS = 553.72) |
| Sub 2-47 | m/z = 609.25(C₄₄H₃₅NS = 609.83) | Sub 2-48 | m/z = 629.22(C₄₆H₃₁NS = 629.82) |
| Sub 2-49 | m/z = 629.22(C₄₆H₃₁NS = 629.82) | Sub 2-50 | m/z = 785.22(C₅₆H₃₅NS₂ = 786.02) |
| Sub 2-51 | m/z = 705.25(C₅₂H₃₅NS = 705.92) | Sub 2-52 | m/z = 685.28(C₅₀H₃₉NS = 685.93) |
| Sub 2-53 | m/z = 729.25(C₅₄H₃₅NS = 729.94) | Sub 2-54 | m/z = 553.19(C₄₀H₂₇NS = 553.72) |
| Sub 2-55 | m/z = 705.25(C₅₂H₃₅NS = 705.92) | Sub 2-56 | m/z = 705.25(C₅₂H₃₅NS = 705.92) |
| Sub 2-57 | m/z = 705.25(C₅₂H₃₅NS = 705.92) | Sub 2-58 | m/z = 567.2(C₄₁H₂₉NS = 567.75) |
| Sub 2-59 | m/z = 563.25(C₄₀H₁₇D₁₀NS = 563.78) | Sub 2-60 | m/z = 629.22(C₄₆H₃₁NS = 629.82) |
| Sub 2-61 | m/z = 787.33(C₅₈H₄₅NS = 788.06) | Sub 2-62 | m/z = 589.28(C₄₂H₃₉NS = 589.84) |
| Sub 2-63 | m/z = 705.25(C₅₂H₃₅NS = 705.92) | Sub 2-64 | m/z = 781.28(C₅₈H₃₉NS = 782.02) |
| Sub 2-65 | m/z = 755.26(C₅₆H₃₇NS = 755.98) | Sub 2-66 | m/z = 477.16(C₃₄H₂₃NS = 477.63) |
| Sub 2-67 | m/z = 705.25(C₅₂H₃₅NS = 705.92) | Sub 2-68 | m/z = 609.25(C₄₄H₃₅NS = 609.83) |
| Sub 2-69 | m/z = 703.25(C₅₂H₃₃NO₂ = 703.84) | Sub 2-70 | m/z = 789.3(C₆₀H₃₉NO = 789.98) |
| Sub 2-71 | m/z = 765.3(C₅₈H₃₉NO = 765.96) | Sub 2-72 | m/z = 669.3(C₅₀H₃₉NO = 669.87) |
| Sub 2-73 | m/z = 685.28(C₅₀H₃₉NS = 685.93) | Sub 2-74 | m/z = 544.25(C₄₀H₂₀D₇NO = 544.7) |
| Sub 2-75 | m/z = 689.27(C₅₂H₃₅NO = 689.86) | Sub 2-76 | m/z = 628.33(C₄₆H₁₆D₁₅NO = 628.85) |
| Sub 2-77 | m/z = 765.3(C₅₈H₃₉NO = 765.96) | Sub 2-78 | m/z = 613.24(C₄₆H₃₁NO = 613.76) |
| Sub 2-79 | m/z = 613.24(C₄₆H₃₁NO = 613.76) | Sub 2-80 | m/z = 793.28(C₅₉H₃₉NS = 794.03) |
| Sub 2-81 | m/z = 626.24(C₄₆H₃₀N₂O = 626.76) | Sub 2-82 | m/z = 626.24(C₄₆H₃₀N₂O = 626.76) |
| Sub 2-83 | m/z = 827.32(C₆₃H₄₁NO = 828.03) | Sub 2-84 | m/z = 635.26(C₄₆H₃₇NS = 635.87) |
| Sub 2-85 | m/z = 511.23(C₃₆H₃₃NS = 511.73) | Sub 2-86 | m/z = 654.23(C₄₇H₃₀N₂O₂ = 654.77) |
| Sub 2-87 | m/z = 769.24(C₅₆H₃₅NOS = 769.96) | Sub 2-88 | m/z = 577.24(C₄₃H₃₁NO = 577.73) |
| Sub 2-89 | m/z = 537.21(C₄₀H₂₇NO = 537.66) | Sub 2-90 | m/z = 553.19(C₄₀H₂₇NS = 553.72) |
| Sub 2-91 | m/z = 461.18(C₃₄H₂₃NO = 461.56) | Sub 2-92 | m/z = 629.22(C₄₆H₃₁NS = 629.82) |
| Sub 2-93 | m/z = 763.29(C₅₈H₃₇NO = 763.94) | Sub 2-94 | m/z = 790.3(C₅₉H₃₈N₂O = 790.97) |
| Sub 2-95 | m/z = 577.2(C₄₂H₂₇NO₂ = 577.68) | Sub 2-96 | m/z = 595.29(C₄₄H₃₇NO = 595.79) |
| Sub 2-97 | m/z = 585.3(C₄₃H₃₉NO = 585.79) | Sub 2-98 | m/z = 799.38(C₆₀H₄₉NO = 800.06) |
| Sub 2-99 | m/z = 784.35(C₅₈H₄₄N₂O = 785) | Sub 2-100 | m/z = 663.26(C₅₀H₃₃NO = 663.82) |
| Sub 2-101 | m/z = 707.3(C₅₂H₂₉D₅N₂O = 707.89) | Sub 2-102 | m/z = 720.31(C₅₃H₄₀N₂O = 720.92) |
| Sub 2-103 | m/z = 677.24(C₅₀H₃₁NO₂ = 677.8) | Sub 2-104 | m/z = 543.26(C₄₀H₃₃NO = 543.71) |
| Sub 2-105 | m/z = 931.33(C₆₇H₄₉NS₂ = 932.26) | Sub 2-106 | m/z = 461.18(C₃₄H₂₃NO = 461.56) |
| Sub 2-107 | m/z = 869.29(C₆₄H₃₉NO₃ = 870.02) | Sub 2-108 | m/z = 577.24(C₄₃H₃₁NO = 577.73) |
| Sub 2-109 | m/z = 659.31(C₄₉H₂₉D₆NO = 659.86) | Sub 2-110 | m/z = 461.18(C₃₄H₂₃NO = 461.56) |
| Sub 2-111 | m/z = 691.33(C₅₀H₄₅NS = 691.98) | Sub 2-112 | m/z = 807.26(C₅₉H₃₇NOS = 808.01) |
| Sub 2-113 | m/z = 511.19(C₃₈H₂₅NO = 511.62) | Sub 2-114 | m/z = 511.19(C₃₈H₂₅NO = 511.62) |
| Sub 2-115 | m/z = 511.19(C₃₈H₂₅NO = 511.62) | Sub 2-116 | m/z = 511.19(C₃₈H₂₅NO = 511.62) |
| Sub 2-117 | m/z = 511.19(C₃₈H₂₅NO = 511.62) | Sub 2-118 | m/z = 527.17(C₃₈H₂₅NS = 527.68) |
| Sub 2-119 | m/z = 516.22(C₃₈H₂₀D₅NO = 516.65) | Sub 2-120 | m/z = 399.16(C₂₉H₂₁NO = 399.49) |
| Sub 2-121 | m/z = 413.18(C₃₀H₂₃NO = 413.52) | Sub 2-122 | m/z = 477.21(C₃₅H₂₇NO = 477.61) |
| Sub 2-123 | m/z = 491.22(C₃₆H₂₉NO = 491.63) | | |

The FD-MS (Field Desorption-Mass Spectrometry) values of compounds P-1 to P-120 of the present invention manufactured according to the synthetic examples are as shown in Table 3.

TABLE 3

| Compound | FD-MS | Compound | FD-MS |
|---|---|---|---|
| P-1 | m/z = 855.31(C₆₄H₄₁NO₂ = 856.04) | P-2 | m/z = 703.25(C₅₂H₃₃NO₂ = 703.84) |
| P-3 | m/z = 703.25(C₅₂H₃₃NO₂ = 703.84) | P-4 | m/z = 703.25(C₅₂H₃₃NO₂ = 703.84) |
| P-5 | m/z = 779.28(C₅₈H₃₇NO₂ = 779.94) | P-6 | m/z = 779.28(C₅₈H₃₇NO₂ = 779.94) |
| P-7 | m/z = 779.28(C₅₈H₃₇NO₂ = 779.94) | P-8 | m/z = 753.27(C₅₆H₃₅NO₂ = 753.9) |
| P-9 | m/z = 779.28(C₅₈H₃₇NO₂ = 779.94) | P-10 | m/z = 779.28(C₅₈H₃₇NO₂ = 779.94) |
| P-11 | m/z = 855.31(C₆₄H₄₁NO₂ = 856.04) | P-12 | m/z = 703.25(C₅₂H₃₃NO₂ = 703.84) |
| P-13 | m/z = 769.24(C₅₆H₃₅NOS = 769.96) | P-14 | m/z = 811.24(C₅₈H₃₇NS₂ = 812.06) |
| P-15 | m/z = 869.29(C₆₄H₃₉NO₃ = 870.02) | P-16 | m/z = 735.21(C₅₂H₃₃NS₂ = 735.96) |
| P-17 | m/z = 720.36(C₅₂H₁₆D₁₇NO₂ = 720.94) | P-18 | m/z = 895.29(C₆₆H₄₁NOS = 896.12) |
| P-19 | m/z = 899.29(C₆₅H₄₁NO₂S = 900.11) | P-20 | m/z = 937.28(C₆₈H₄₃NS₂ = 938.22) |
| P-21 | m/z = 708.28(C₅₂H₂₈D₅NO₂ = 708.87) | P-22 | m/z = 758.3(C₅₆H₃₀D₅NO₂ = 758.93) |
| P-23 | m/z = 773.33(C₅₇H₄₃NO₂ = 773.98) | P-24 | m/z = 789.35(C₅₈H₂₇D₁₀NO₂ = 790) |
| P-25 | m/z = 703.25(C₅₂H₃₃NO₂ = 703.84) | P-26 | m/z = 1111.37(C₈₂H₄₉NO₄ = 1112.3) |
| P-27 | m/z = 703.25(C₅₂H₃₃NO₂ = 703.84) | P-28 | m/z = 875.44(C₆₄H₂₁D₂₀NO₂ = 876.16) |
| P-29 | m/z = 931.35(C₇₀H₄₅NO₂ = 932.14) | P-30 | m/z = 1057.39(C₈₀H₅₁NO₂ = 1058.29) |
| P-31 | m/z = 1017.45(C₇₆H₅₉NO₂ = 1018.31) | P-32 | m/z = 1097.39(C₈₂H₅₁NO₃ = 1098.31) |
| P-33 | m/z = 931.35(C₇₀H₄₅NO₂ = 932.14) | P-34 | m/z = 945.32(C₇₀H₄₃NO₃ = 946.12) |
| P-35 | m/z = 865.38(C₆₄H₃₁D₁₀NO₂ = 866.1) | P-36 | m/z = 843.39(C₆₂H₂₅D₁₄NO₂ = 844.08) |
| P-37 | m/z = 931.35(C₇₀H₄₅NO₂ = 932.14) | P-38 | m/z = 1159.44(C₈₈H₅₇NO₂ = 1160.43) |
| P-39 | m/z = 1111.37(C₈₂H₄₉NO₄ = 1112.3) | P-40 | m/z = 829.3(C₆₂H₃₉NO₂ = 830) |
| P-41 | m/z = 963.3(C₇₀H₄₅NS₂ = 964.26) | P-42 | m/z = 745.27(C₅₂H₂₃D₁₀NS₂ = 746.02) |
| P-43 | m/z = 1069.29(C₇₆H₄₇NS₃ = 1070.4) | P-44 | m/z = 867.3(C₆₂H₄₅NS₂ = 868.17) |
| P-45 | m/z = 917.22(C₆₄H₃₉NS₃ = 918.2) | P-46 | m/z = 887.27(C₆₄H₄₁NS₂ = 888.16) |
| P-47 | m/z = 923.36(C₆₆H₅₃NS₂ = 924.28) | P-48 | m/z = 1039.33(C₇₆H₄₉NS₂ = 1040.36) |
| P-49 | m/z = 887.27(C₆₄H₄₁NS₂ = 888.16) | P-50 | m/z = 1043.27(C₇₄H₄₅NS₃ = 1044.36) |
| P-51 | m/z = 1115.36(C₈₂H₅₃NS₂ = 1116.45) | P-52 | m/z = 999.39(C₇₂H₅₇NS₂ = 1000.37) |
| P-53 | m/z = 987.3(C₇₂H₄₅NS₂ = 988.28) | P-54 | m/z = 867.3(C₆₂H₄₅NS₂ = 868.17) |
| P-55 | m/z = 1039.33(C₇₆H₄₉NS₂ = 1040.36) | P-56 | m/z = 1039.33(C₇₆H₄₉NS₂ = 1040.36) |
| P-57 | m/z = 1039.33(C₇₆H₄₉NS₂ = 1040.36) | P-58 | m/z = 906.32(C₆₅H₃₈D₅NS₂ = 907.22) |
| P-59 | m/z = 826.33(C₅₈H₂₂D₁₅NS₂ = 827.15) | P-60 | m/z = 963.3(C₇₀H₄₅NS₂ = 964.26) |
| P-61 | m/z = 1105.43(C₈₂H₅₉NOS = 1106.44) | P-62 | m/z = 887.42(C₆₄H₅₇NOS = 888.23) |
| P-63 | m/z = 1023.35(C₇₆H₄₉NOS = 1024.29) | P-64 | m/z = 1099.38(C₈₂H₅₃NOS = 1100.39) |
| P-65 | m/z = 1225.43(C₉₂H₅₉NOS = 1226.55) | P-66 | m/z = 845.28(C₆₂H₃₉NOS = 846.06) |
| P-67 | m/z = 947.32(C₇₀H₄₅NOS = 948.2) | P-68 | m/z = 963.45(C₇₀H₆₁NOS = 964.32) |
| P-69 | m/z = 1189.4(C₈₈H₅₅NO₂S = 1190.47) | P-70 | m/z = 1199.42(C₉₀H₅₇NOS = 1200.51) |
| P-71 | m/z = 1099.38(C₈₂H₅₃NOS = 1100.39) | P-72 | m/z = 983.42(C₇₂H₅₇NOS = 984.31) |
| P-73 | m/z = 927.35(C₆₈H₄₉NOS = 928.21) | P-74 | m/z = 935.39(C₆₈H₂₉D₁₄NOS = 936.24) |
| P-75 | m/z = 1023.35(C₇₆H₄₉NOS = 1024.29) | P-76 | m/z = 967.45(C₇₀H₂₅D₂₀NOS = 968.32) |
| P-77 | m/z = 1099.38(C₈₂H₅₃NOS = 1100.39) | P-78 | m/z = 1023.35(C₇₆H₄₉NOS = 1024.29) |
| P-79 | m/z = 1023.35(C₇₆H₄₉NOS = 1024.29) | P-80 | m/z = 1035.35(C₇₇H₄₉NOS = 1036.31) |
| P-81 | m/z = 1044.37(C₇₈H₄₈N₂O₂ = 1045.25) | P-82 | m/z = 962.39(C₇₁H₅₀N₂O₂ = 963.19) |
| P-83 | m/z = 1145.42(C₈₇H₅₅NO₂ = 1146.4) | P-84 | m/z = 1195.42(C₈₈H₆₁NS₂ = 1196.58) |
| P-85 | m/z = 919.35(C₆₆H₄₉NO₂S = 920.18) | P-86 | m/z = 1116.43(C₈₂H₅₆N₂O₃ = 1117.36) |
| P-87 | m/z = 1011.32(C₇₄H₄₅NO₂S = 1012.24) | P-88 | m/z = 953.42(C₇₁H₅₅NO₂ = 954.23) |
| P-89 | m/z = 861.36(C₆₄H₄₇NO₂ = 862.08) | P-90 | m/z = 887.27(C₆₄H₄₁NS₂ = 888.16) |
| P-91 | m/z = 829.3(C₆₂H₃₉NO₂ = 830) | P-92 | m/z = 1185.4(C₈₉H₅₅NOS = 1186.49) |
| P-93 | m/z = 1005.36(C₇₆H₄₇NO₂ = 1006.22) | P-94 | m/z = 1260.47(C₉₅H₆₀N₂O₂ = 1261.54) |
| P-95 | m/z = 971.34(C₇₂H₄₅NO₃ = 972.16) | P-96 | m/z = 1035.49(C₇₇H₅₃D₆NO₂ = 1036.36) |
| P-97 | m/z = 993.45(C₇₄H₅₉NO₂ = 994.29) | P-98 | m/z = 1055.47(C₇₉H₆₁NO₂ = 1056.36) |
| P-99 | m/z = 1026.42(C₇₆H₅₄N₂O₂ = 1027.28) | P-100 | m/z = 1122.38(C₈₃H₅₀N₂O₃ = 1123.32) |
| P-101 | m/z = 1187.53(C₈₈H₄₁D₁₅N₂O₂ = 1188.52) | P-102 | m/z = 1056.47(C₇₈H₆₀N₂O₂ = 1057.35) |
| P-103 | m/z = 1023.37(C₇₆H₄₉NO₃ = 1024.23) | P-104 | m/z = 1079.42(C₈₀H₅₇NOS = 1080.4) |
| P-105 | m/z = 1267.48(C₉₂H₆₉NOS₂ = 1268.69) | P-106 | m/z = 703.25(C₅₂H₃₃NO₂ = 703.84) |
| P-107 | m/z = 1111.37(C₈₂H₄₉NO₄ = 1112.3) | P-108 | m/z = 1051.35(C₇₇H₄₉NO₂S = 1052.3) |
| P-109 | m/z = 1099.51(C₈₂H₄₅D₁₂NO₂ = 1100.44) | P-110 | m/z = 703.25(C₅₂H₃₃NO₂ = 703.84) |
| P-111 | m/z = 1169.5(C₈₅H₇₁NS₂ = 1170.63) | P-112 | m/z = 1063.35(C₇₈H₄₉NO₂S = 1064.31) |
| P-113 | m/z = 753.27(C₅₆H₃₅NO₂ = 753.9) | P-114 | m/z = 803.28(C₆₀H₃₇NO₂ = 803.96) |
| P-115 | m/z = 803.28(C₆₀H₃₇NO₂ = 803.96) | P-116 | m/z = 753.27(C₅₆H₃₅NO₂ = 753.9) |
| P-117 | m/z = 753.27(C₅₆H₃₅NO₂ = 753.9) | P-118 | m/z = 769.24(C₅₆H₃₅NOS = 769.96) |
| P-119 | m/z = 769.24(C₅₆H₃₅NOS = 769.96) | P-120 | m/z = 758.3(C₅₆H₃₀D₅NO₂ = 758.93) |
| P-121 | m/z = 641.24(C₄₇H₃₁NO₂ = 641.77) | P-122 | m/z = 655.25(C₄₈H₃₃NO₂ = 655.8) |
| P-123 | m/z = 719.28(C₅₃H₃₇NO₂ = 719.88) | P-124 | m/z = 733.3(C₅₄H₃₉NO₂ = 733.91) |
| P-125 | m/z = 795.26(C₅₈H₃₇NOS = 796) | | |

Meanwhile, exemplary synthesis examples of the present invention represented by Formula 1 have been described, but these are all based on the Buchwald-Hartwig cross coupling reaction, Miyaura boration reaction, Suzuki cross-coupling reaction, Intramolecular acid-induced cyclization reaction (J. mater. Chem. 1999, 9, 2095), Pd(II)-catalyzed oxidative cyclization reaction (Org. Lett. 2011, 13, 5504), and PPh₃-mediated reductive cyclization reaction (J. Org. Chem. 2005, 70, 5014), and it will be easily understood by those skilled in the art that the reaction proceeds even when other substituents defined in Formula 1 are bonded in addition to the substituents specified in the specific synthesis examples.

### Manufacturing Evaluation of Organic Electroluminescent Devices

### [Example 1] Red Organic Electroluminescent Device (Emitting-Auxiliary Layer)

Compound A and Compound B were used on the ITO layer (anode) formed on a glass substrate, and Compound B was doped at a weight ratio of 98:2 to form a hole injection layer with a thickness of 10 nm. Then, Compound A was vacuum-deposited on the hole injection layer with a thickness of 110 nm to form a hole transport layer.

Next, the compound P-2 of the present invention was vacuum-deposited on the hole transport layer to a thickness of 10 nm to form an emitting-auxiliary layer. Thereafter, compound D-R was used as the host material of the emitting layer, and bis-(1-phenylisoquinolyl) iridium (III) acetylacetonate (hereinafter abbreviated as ‘(piq)₂Ir(acac)’) was used as the dopant material, and the dopant was doped so that the weight ratio of the host and the dopant was 95:5, thereby forming an emitting layer with a thickness of 30 nm.

Next, compound E was vacuum-deposited on the emitting layer to form a hole-blocking layer with a thickness of 10 nm, and a mixture of compound F and compound G at a weight ratio of 5:5 was used to form an electron transport layer with a thickness of 30 nm on the hole-blocking layer. Thereafter, compound G was deposited on the electron transport layer to form an electron injection layer with a thickness of 0.2 nm, and then Al was deposited to form a cathode with a thickness of 150 nm.
compound A: N-([1,1′-biphenyl]-4-yl)-9,9-dimethyl-N-(4-(9-phenyl-9H-carbazol-3-yl)phenyl)-9H-fluoren-2-amine
compound B: 4,4′,4″-((1E,1′E,1″E)-cyclopropane-1,2,3-triylidenetris(cyanomethaneylylidene))tris(2,3,5,6-tetrafluorobenzonitrile)
compound D-R: 14-(4-phenylquinazolin-2-yl)-14H-benzo[c]benzo[4,5]thieno[2,3-a]carbazole
compound E: 2-(4′-(9,9-dimethyl-9H-fluoren-2-yl)-[1,1′-biphenyl]-3-yl)-4,6-diphenyl-1,3,5-triazine
compound F: 2,7-bis(4-(4,6-diphenyl-1,3,5-triazin-2-yl)phenyl)naphthalene
compound G: (8-quinolinolato)lithium

### [Example 1] to [Example 18]

An organic electroluminescent device was manufactured in the same manner as in Example 1, except that the compound of the present invention described in Table 4 was used instead of the compound P-2 of the present invention as an emitting auxiliary layer material.

### [Comparative Example 1] to [Comparative Example 6]

An organic electroluminescent device was manufactured in the same manner as Example 1, except that Comparative Compounds A to F were used instead of Compound P-2 of the present invention as an emitting-auxiliary layer material.

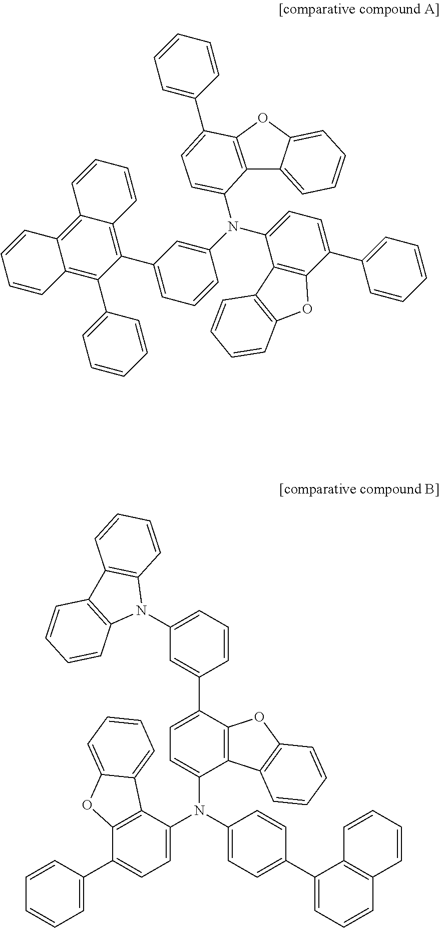 [SMILES: c1ccc(-c2c(-c3cccc(N(c4ccc(-c5ccccc5)c5oc6ccccc6c45)c4ccc(-c5ccccc5)c5oc6ccccc6c45)c3)c3ccccc3c3ccccc23)cc1 ; c1ccc(-c2ccc(N(c3ccc(-c4cccc5ccccc45)cc3)c3ccc(-c4cccc(-n5c6ccccc6c6ccccc65)c4)c4oc5ccccc5c34)c3c2oc2ccccc23)cc1]

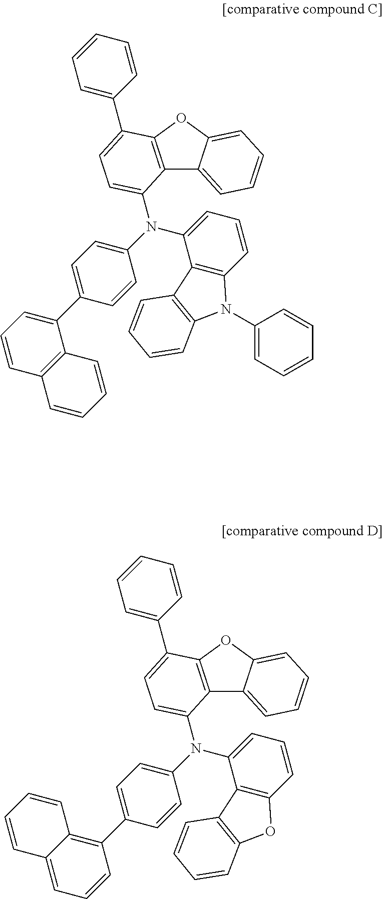 [SMILES: c1ccc(-c2ccc(N(c3ccc(-c4cccc5ccccc45)cc3)c3cccc4c3c3ccccc3n4-c3ccccc3)c3c2oc2ccccc23)cc1 ; c1ccc(-c2ccc(N(c3ccc(-c4cccc5ccccc45)cc3)c3cccc4oc5ccccc5c34)c3c2oc2ccccc23)cc1]

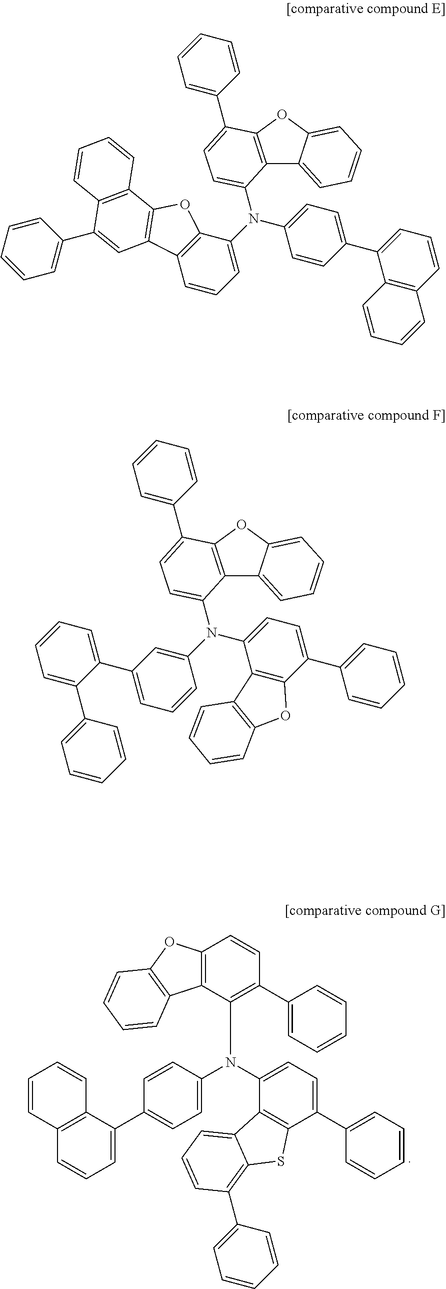 [SMILES: c1ccc(-c2cc3c4cccc(N(c5ccc(-c6cccc7ccccc67)cc5)c5ccc(-c6ccccc6)c6oc7ccccc7c56)c4oc3c3ccccc23)cc1 ; c1ccc(-c2ccccc2-c2cccc(N(c3ccc(-c4ccccc4)c4oc5ccccc5c34)c3ccc(-c4ccccc4)c4oc5ccccc5c34)c2)cc1 ; c1ccc(-c2ccc3oc4ccccc4c3c2N(c2ccc(-c3cccc4ccccc34)cc2)c2ccc(-c3ccccc3)c3sc4c(-c5ccccc5)cccc4c23)cc1]

The electroluminescence (EL) characteristics were measured using PR-650 from Photoresearch by applying a forward bias DC voltage to the organic electroluminescence devices manufactured by Examples 1 to 18 of the present invention and Comparative Examples 1 to 6. As a result of the measurement, the T95 lifespan was measured using a lifespan measuring device manufactured by Max Science at a standard brightness of 2,500 cd/m². Table 4 shows the results of the device fabrication and evaluation.

The measuring apparatus can evaluate the performance of new materials compared to comparative compounds under identical conditions, without being affected by possible daily fluctuations in deposition rate, vacuum quality or other parameters.

During the evaluation, one batch contains 4 identically prepared OLEDs including a comparative compound, and the performance of a total of 12 OLEDs is evaluated in 3 batches, so the value of the experimental results obtained in this way indicates statistical significance.

TABLE 4

| | | Driving | Current | | | |
|---|---|---|---|---|---|---|
| | | Voltage | Density | Brightness | Efficiency | Lifetime |
| | Compound | (V) | (mA/cm²) | (cd/m²) | (cd/A) | T(95) |
| Comparative | Comparative | 5.2 | 14.9 | 2500.0 | 18.5 | 92.5 |
| example (1) | compound A | | | | | |
| Comparative | Comparative | 5.1 | 12.8 | 2500.0 | 19.5 | 97.5 |
| example (2) | compound B | | | | | |
| Comparative | Comparative | 5.4 | 14.2 | 2500.0 | 17.6 | 82.5 |
| example (3) | compound C | | | | | |
| Comparative | Comparative | 5.1 | 12.8 | 2500.0 | 19.6 | 91.5 |
| example (4) | compound D | | | | | |
| Comparative | Comparative | 5.3 | 15.5 | 2500.0 | 16.1 | 87.1 |
| example (5) | compound E | | | | | |
| Comparative | Comparative | 5.2 | 13.7 | 2500.0 | 18.3 | 91.2 |
| example (6) | compound F | | | | | |
| Comparative | Comparative | 5.2 | 13.0 | 2500.0 | 19.3 | 94.5 |
| example (7) | compound G | | | | | |
| Example (1) | P-2 | 4.5 | 7.6 | 2500.0 | 32.8 | 112.4 |
| Example (2) | P-11 | 4.4 | 7.5 | 2500.0 | 33.4 | 116.8 |
| Example (3) | P-22 | 4.3 | 7.0 | 2500.0 | 35.6 | 120.2 |
| Example (4) | P-23 | 4.1 | 6.6 | 2500.0 | 37.9 | 126.8 |
| Example (5) | P-25 | 4.0 | 6.3 | 2500.0 | 39.4 | 127.9 |
| Example (6) | P-26 | 4.1 | 6.6 | 2500.0 | 38.0 | 127.1 |
| Example (7) | P-34 | 4.2 | 6.5 | 2500.0 | 38.3 | 126.0 |
| Example (8) | P-40 | 4.5 | 8.0 | 2500.0 | 31.4 | 112.6 |
| Example (9) | P-54 | 4.3 | 6.8 | 2500.0 | 36.5 | 124.0 |
| Example (10) | P-62 | 4.4 | 7.3 | 2500.0 | 34.1 | 118.5 |
| Example (11) | P-78 | 4.5 | 7.7 | 2500.0 | 32.5 | 115.9 |
| Example (12) | P-83 | 4.5 | 7.6 | 2500.0 | 32.9 | 115.4 |
| Example (13) | P-88 | 4.4 | 7.2 | 2500.0 | 34.7 | 118.8 |
| Example (14) | P-89 | 4.5 | 8.3 | 2500.0 | 30.2 | 111.5 |
| Example (15) | P-99 | 4.2 | 6.6 | 2500.0 | 37.8 | 126.2 |
| Example (16) | P-115 | 4.3 | 7.0 | 2500.0 | 35.9 | 123.4 |
| Example (17) | P-117 | 4.0 | 6.4 | 2500.0 | 39.0 | 128.2 |
| Example (18) | P-120 | 4.4 | 7.6 | 2500.0 | 33.1 | 117.1 |
| Example (19) | P-125 | 4.1 | 6.5 | 2500.0 | 38.6 | 127.0 |

Comparative compounds A to F are similar to the compounds of the present invention in that they have a tertiary amine structure (hereinafter, the parent nucleus) in which a substituent is substituted at the 4-position of dibenzofuran or dibenzothiophene and an amine group is bonded at the 1-position, but they differ from the compounds of the present invention in that one of the remaining substituents of the amine is phenyl substituted with naphthyl, and another is dibenzofuran or dibenzothiophene in which a substituent is further substituted at a specific position of the ring bonded to the amine.

Comparative compounds A and B are similar to the compound of the present invention in that one of the remaining amine substituents other than the parent nucleus includes a dibenzofuran group, but comparative compound A differs from the compound of the present invention in that one of the substituents of the amine group includes phenanthrene, and comparative compound B differs from the compound of the present invention in that carbazole is further substituted at the R1 position of the parent nucleus. In order to confirm the difference in energy levels of compounds due to these structural differences, data measured using the DFT method (B3LYP/6-31g(D)) of the Gaussian program for comparative compounds A and B and the compound P-25 of the present invention, which has the most similar structure, are as shown in Table 5.

TABLE 5

| Compound | LUMO(eV) |
|---|---|
| P-25 | −1.28 |
| Comparative compound A | −1.37 |
| Comparative compound B | −1.40 |

As can be seen from the results in Table 5, it can be confirmed that the LUMO value of compound P-25 of the present invention is shallower than the LUMO Energy Level (hereinafter, LUMO) values of comparative compounds A and B. As a result, when the compound of the present invention is applied to an element, the electron blocking effect in the emitting-auxiliary layer is increased compared to comparative compounds A and B, and thus the lifespan of the device is thought to be significantly improved.

Next, in the case of comparative compound C, it is similar to the compound of the present invention in that one of the remaining amine substituents other than the parent nucleus includes a phenyl group substituted with naphthyl, but comparative compound C is different from the compound of the present invention in that one of the substituents of the amine group is carbazole. In order to confirm the difference in energy levels of the compounds due to these structural differences, data measured using the DFT method (B3LYP/6-31g(D)) of the Gaussian program for the comparative compound C and the compound P-25 of the present invention, which has the most similar structure, are as shown in Table 6.

TABLE 6

| Compound | HOMO(eV) |
|---|---|
| P-25 | −5.08 |
| Comparative compound C | −4.96 |

As can be seen from the results in Table 6, it can be confirmed that the HOMO value of the compound P-25 of the present invention is deeper than the energy level (hereinafter, HOMO) value of the comparative compound C. As a result, when the compound of the present invention is applied to a device, hole transfer from the emitting auxiliary layer to the emitting layer becomes easier than with the comparative compound C, so the hole accumulation in the device decreases, and accordingly, the charge balance increases, and the efficiency and lifespan of the device are thought to be significantly improved.

Next, in the case of comparative compound D, it is similar to the compound of the present invention in that one of the remaining amine substituents other than the parent nucleus includes a phenyl group substituted with a naphthyl group, but comparative compound D is different from the compound of the present invention in that the remaining substituent of the amine group, a 3-condensed ring dibenzofuran, is not additionally substituted with a substituent. In order to confirm the change in BDE of the compound due to such structural difference, the data measured using the DFT method (B3LYP/6-31g(D)) of the Gaussian program for the compound P-25 of the present invention, which has a high similarity to the comparative compound D, are as shown in Table 7.

TABLE 7

| Compound | BDE(eV) |
|---|---|
| P-25 | 3.118 |
| Comparative compound D | 2.869 |

As can be seen from the results in Table 7, when comparing the lowest BDE value between carbon and hydrogen of P-25, a compound of the present invention in which a phenyl group is additionally substituted at the 4th position of dibenzofuran in the amine group, with the lowest BDE value between carbon and hydrogen of comparative compound D in which dibenzofuran in which a substituent is not substituted in the amine group, the BDE value is greater. This result can be seen as indicating that the structural stability of the compound of the present invention in which a phenyl group is additionally substituted in dibenzofuran is superior, and thus, it is thought that the lifespan is significantly improved.

Next, in the case of Comparative Compound E, it is similar to the compound of the present invention in that one of the amine substituents includes a phenyl group substituted with a naphthyl group, and the other of the amine substituents is substituted with a naphthobenzofuran of a 4-condensed ring, but the Comparative Compound differs from the compound of the present invention in that the substituent of the 4-condensed ring is not substituted on the ring bonded to the amine. In order to confirm the change in the Dipole momonet of the compound due to such structural difference, the data measured using the DFT method (B3LYP/6-31g(D)) of the Gaussian program for the compound P-115 of the present invention, which has a high similarity to the comparative compound E, are as shown in Table 8.

TABLE 8

| Compound | Dipole moment(D) |
|---|---|
| P-115 | 1.4880 |
| Comparative compound E | 1.3674 |

As can be seen from the results in Table 8, it can be confirmed that the dipole moment values of the comparative compound E and the compound of the present invention are significantly different. To explain in more detail, the compound of the present invention, which includes an additional substituent on the ring in which the 4-condensed ring naphthobenzofuran and the amine are bonded, has a larger dipole moment value than the comparative compound E. As a result, when the compound of the present invention is applied to a device, the mobility of holes is improved compared to the comparative compounds due to the stronger van der Waals interaction within the molecule, and as a result, the charge balance of the device is improved, and thus the performance of the device is also judged to be significantly improved.

Next, in the case of comparative compound F, it is different from the present invention in that a relatively bulky terphenyl moiety is substituted instead of the naphthyl-substituted phenyl moiety, which is an essential component of the compound of the present invention. As a result, when comparative compound F is applied to a device, it is judged that the compound of the present invention, which has a relatively small molecular volume, has a relatively close intermolecular distance, so that hole movement and hole injection are significantly improved compared to the comparative compound, and as a result, it is judged that the hole transport capability of the device is improved.

Finally, in the case of comparative compound G, it is similar to the compound of the present invention in that one of the remaining amine substituents other than the parent nucleus includes a phenyl group substituted with a naphthyl group, but comparative compound G is different from the compound of the present invention in that an amine group is substituted at position 1 of dibenzofuran and a substituent is substituted at position 2. As a result, the comparative compound G has a greater steric hinderance than the compound of the present invention due to the structural features described above, and as a result, the molecular structure itself becomes more distorted than that of the compound of the present invention. Therefore, it is believed that when the compound of the present invention, which has a relatively greater planarity, is applied to a device, the hole transport ability is improved compared to the comparative compound G, and thus the performance of the device is affected.

These results suggest that even if the molecular components are similar, the properties of the compound, such as hole characteristics of molecules, light efficiency characteristics, energy levels, hole injection and mobility characteristics, charge balance of holes and electrons, volume density, and intermolecular distance, can differ significantly to an extent that is difficult to predict depending on the type and position of the substituted substituent, and that the performance of the device can vary not only due to the composition of a single compound but also due to complex factors.

In the case of an emitting-auxiliary layer, the relationship between the hole transport layer and the emitting layer (host) must be understood. Therefore, even if a similar core is used, it would be very difficult for even a person skilled in the art to infer the characteristics exhibited by the emitting-auxiliary layer using the compound of the present invention.

In addition, the evaluation results of the above-described device fabrication described the device characteristics in which the compound of the present invention was applied only to the emitting-auxiliary layer, but the compound of the present invention may be applied to the hole transport layer or may be used in both the hole transport layer and the emitting-auxiliary layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment.

The scope of the present invention shall be construed on the basis of the accompanying claims, and it shall be construed that all of the technical ideas included within the scope equivalent to the claims belong to the present invention.

1. A compound represented by Formula 1: 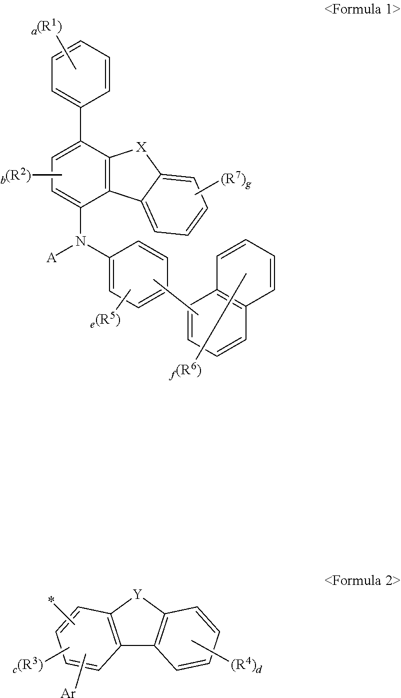
wherein:
X and Y are independently O or S,
R¹ is selected from the group consisting of hydrogen; deuterium; a C₁-C₅₀ alkyl group; a C₃-C₆₀ aliphatic ring; a fluorenyl group; or a C₆-C₆₀ aryl group, and a plurality of R¹ may be bonded to each other to form a ring,
R², R³, R⁴, R⁵, R⁶ and R⁷ are independently of each other selected from the group consisting of hydrogen; deuterium; a C₆-C₆₀ aryl group; a fluorenyl group; a C₂-C₆₀ heterocyclic group including at least one heteroatom of O, N, S, Si and P; a C₃-C₆₀ aliphatic ring; and a C₁-C₅₀ alkyl group, and a plurality of adjacent groups thereof may be bonded to each other to form a ring,
a is an integer of 0 to 5, b and c are an integer of 0 to 2, d, e and g are each an integer of 0 to 4, f is an integer of 0 to 7,
* indicates a position to be bonded, and
A is a substituent represented by Formula 2,
Ar is a C₁-C₅₀ alkyl group; a C₃-C₆₀ aliphatic ring; a fluorenyl group; or a C₆-C₆₀ aryl group,
wherein the aryl group, heterocyclic group, fluorenyl group, and alkyl group may be substituted with one or more substituents selected from the group consisting of cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic ring; a C₇-C₂₀ arylalkyl group; a C₈-C₂₀ arylalkenyl group; and a C₇-C₂₀ alkylaryl group, and the hydrogen of these substituents may be further substituted with one or more deuterium, and the substituents may be bonded to each other to form a saturated or unsaturated ring, wherein the term ‘ring’ means a C₃-Coo aliphatic ring or a C₆-C₆₀ aromatic ring or a C₂-C₆₀ heterocyclic group or a fused ring formed by the combination thereof, and
wherein Formula 2-A is excluded from Formula 2:

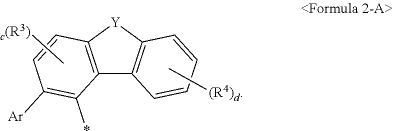

2. The compound according to claim 1, wherein Formula 1 is represented by any one of Formulas 1-1 to 1-6: 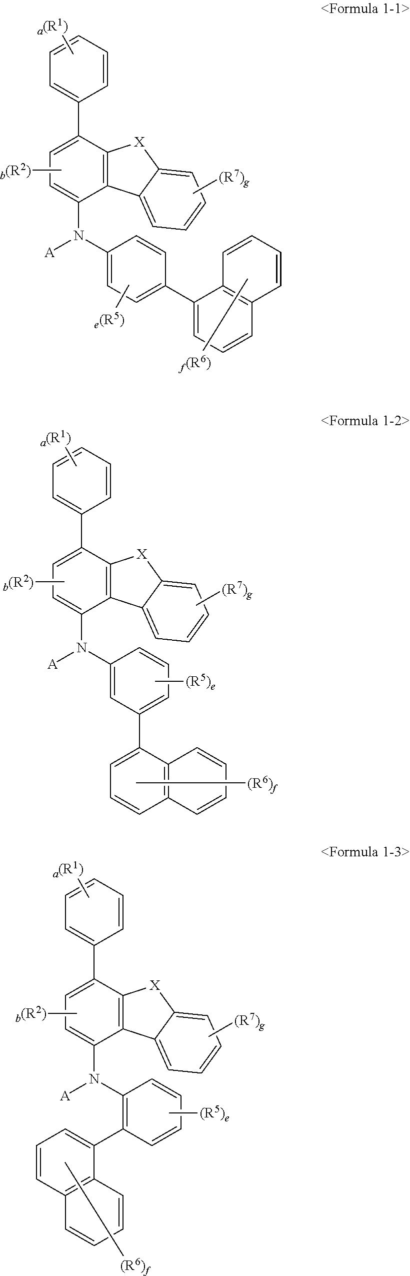 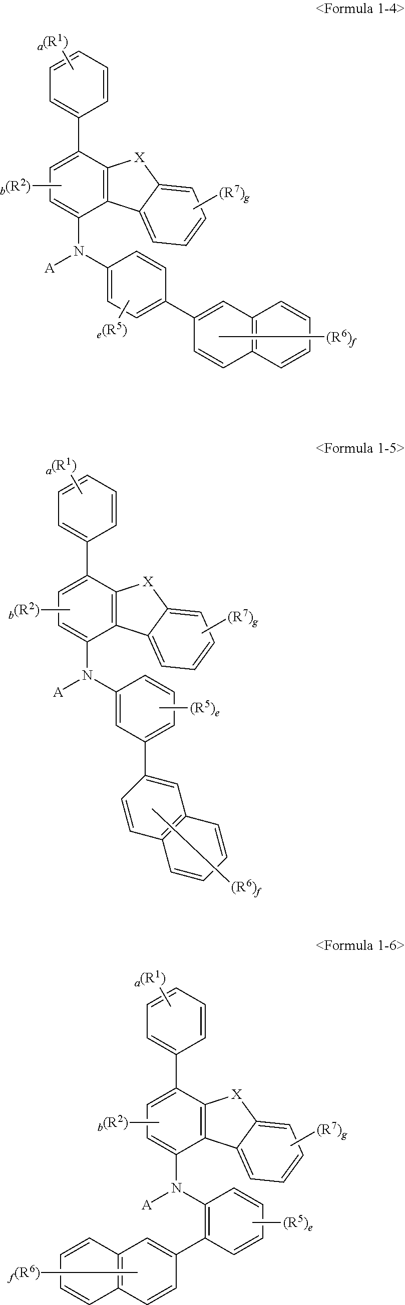
wherein, X, A, R¹, R², R⁵, R$, R⁷, a, b, e, f and g are the same as defined in claim 1.

3. The compound according to claim 1, wherein Formula 2 is represented by any one of Formulas 2-1 to 2-4: 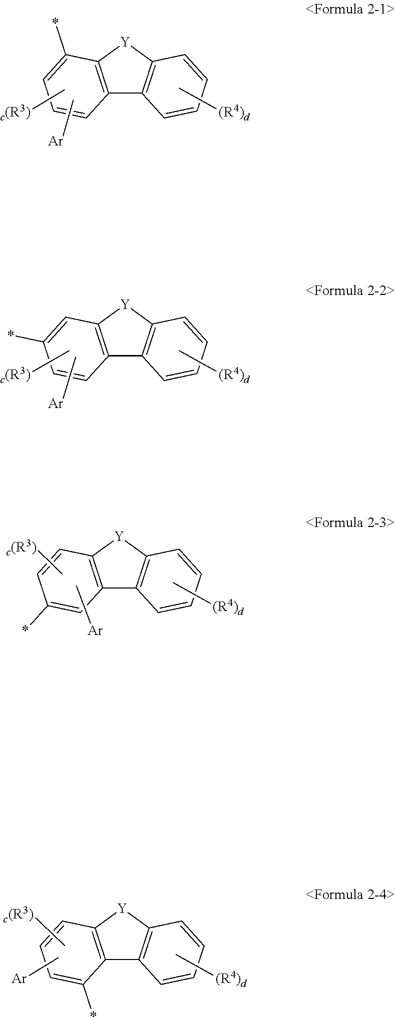
wherein, Ar, Y, R³, R⁴, c, d and * are the same as defined in claim 1.

4. The compound according to claim 1, wherein Ar is represented by any one of Formulas Ar-1 to Ar-15: 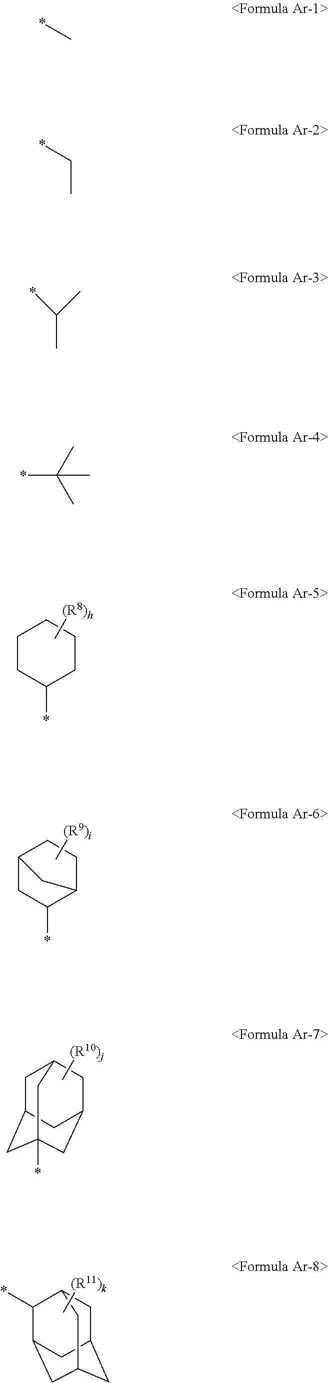 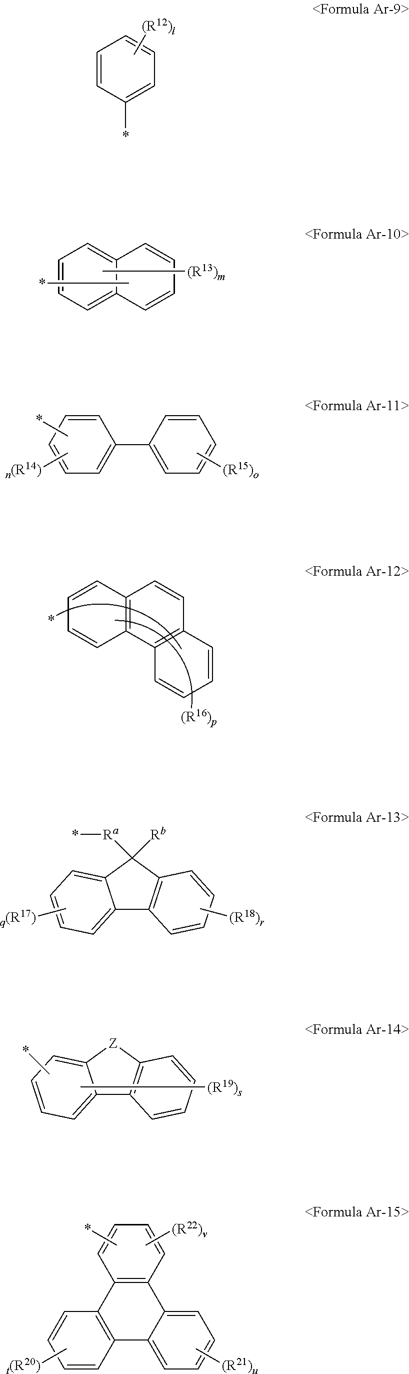
wherein:
R⁸, R⁹, R¹⁰, R¹¹, R¹², R¹³, R¹⁴, R¹⁵, R¹⁶, R¹⁷, R¹⁸, R¹⁹, R²⁰, R²¹ and R²² are independently of each other selected from the group consisting of hydrogen; deuterium; a C₆-C₄₀ aryl group; a fluorenyl group; a C₂-C₄₀ heterocyclic group including at least one heteroatom of O, N, S, Si and P; a C₃-C₄₀ aliphatic ring; a C₁-C₂₀ alkyl group; a C₂-C₂₀ alkenyl group; a C₂-C₂₀ alkynyl group; a C₁-C₃₀ alkoxyl group; and a C₆-C₃₀ an aryloxy group; and a plurality of adjacent groups thereof may be bonded to each other to form an aromatic ring,
Z is CR^{c}R^{d},
R^{a}, R^{b}, R^{c}, R^{d} are independently of each other selected from the group consisting of hydrogen; deuterium; a C₆-C₂₀ aryl group; a C₆-C₂₀ aryl group substituted with deuterium; a fluorenyl group; a C₂-C₂₀ heterocyclic group; a C₅-C₂₀ cycloalkyl group; a C₁-C₂₀ alkyl group; and a C₂-C₂₀ alkenyl group; and a plurality of adjacent R^{a} and R^{b}, or a plurality of adjacent R^{c} and R^{d} may be bonded to each other to form an aromatic ring,
h, l and o are each an integer of 0 to 5, i is an integer of 0 to 6, j, k and p are each an integer of 0 to 9, m is an integer of 0 to 7, n, q, r, t and u are each an integer of 0 to 4, s is an integer of 0 to 7 and v is an integer of 0 to 3, and
Formulas Ar-1 to Ar-4 may be substituted with deuterium.

5. An organic electronic element comprising a first electrode; a second electrode; and an organic material layer formed between the first electrode and the second electrode, wherein the organic material layer comprises a single compound or 2 or more compounds represented by Formula 1 of claim 1.

6. The organic electronic element according to claim 5, wherein the organic material layer comprises at least one of a hole injection layer, a hole transport layer, an emitting-auxiliary layer, an emitting layer, an electron transport-auxiliary layer, an electron transport layer and an electron injection layer.

7. The organic electronic element according to claim 5, wherein the organic material layer is an emitting-auxiliary layer.

8. The organic electronic element according to claim 5, further comprising a light efficiency enhancing layer formed on at least one surface of the first electrode and the second electrode, the surface being opposite to the organic material layer.

9. The organic electronic element according to claim 5, wherein the organic material layer comprises 2 or more stacks including a hole transport layer, an emitting layer, and an electron transport layer sequentially formed on a first electrode.

10. The organic electronic element according to claim 9, wherein the organic material layer further comprises a charge generation layer formed between the 2 or more stacks.

11. An electronic device comprising a display device comprising the organic electronic element of claim 5; and a control unit for driving the display device.

12. The electronic device according to claim 11, wherein the organic electronic element is at least one of an OLED, an organic solar cell, an organic photo conductor (OPC), organic transistor (organic TFT) and an element for monochromic or white illumination.

13. A method for reusing a compound of Formula 1 of claim 1 comprising:
recovering a crude organic light emitting material comprising a compound of Formula 1 of claim 1 from a deposition apparatus used in a process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.
